SEQUENCE LISTING (40 sequences)

SEQ ID NO: 1 - protein, 15 aa, SARS-CoV-2
DSFKEELDKYFKNHT

SEQ ID NO: 2 - protein, 15 aa, SARS-CoV-2
CFTNVYADSFVIRGD

SEQ ID NO: 3 - protein, 15 aa, SARS-CoV-2
PDDFTGCVIAWNSNN

SEQ ID NO: 4 - protein, 15 aa, SARS-CoV-2
FRKSNLKPFERDIST

SEQ ID NO: 5 - protein, 15 aa, SARS-CoV-2
RDISTEIYQAGSTPC

SEQ ID NO: 6 - protein, 15 aa, SARS-CoV-2
EIYQAGSTPCNGVEG

SEQ ID NO: 7 - protein, 15 aa, SARS-CoV-2
NKKFLPFQQFGRDIA

SEQ ID NO: 8 - protein, 15 aa, SARS-CoV-2
PFQQFGRDIADTTDA

SEQ ID NO: 9 - protein, 15 aa, SARS-CoV-2
ECDIPIGAGICASYQ

SEQ ID NO: 10 - protein, 15 aa, SARS-CoV-2
AENSVAYSNNSIAIP

SEQ ID NO: 11 - protein, 15 aa, SARS-CoV-2
AYSNNSIAIPTNFTI

SEQ ID NO: 12 - protein, 15 aa, SARS-CoV-2
KQIYKTPPIKDFGGF

SEQ ID NO: 13 - protein, 14 aa, SARS-CoV-2
TPPIKDFGGFNFSQ

SEQ ID NO: 14 - protein, 15 aa, SARS-CoV-2
VLNDILSRLDKVEAE

SEQ ID NO: 15 - protein, 15 aa, SARS-CoV-2
LQSLQTYVTQQLIRA

SEQ ID NO: 16 - protein, 15 aa, SARS-CoV-2
LQPELDSFKEELDKY

SEQ ID NO: 17 - protein, 1256 aa, Artificial Sequence
  Synthetic Polypeptide
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPSGRLVPRGSPGSGYIPEAPRDGQAYVRKDGEWVLLSTFLGHHHHHH

SEQ ID NO: 18 - protein, 243 aa, Artificial Sequence
  Synthetic Polypeptide
MFVFLVLLPLVSSQRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFHHHHHH

SEQ ID NO: 19 - protein, 223 aa, SARS-CoV-2
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNF

SEQ ID NO: 20 - protein, 72 aa, SARS-CoV-2
NSNNLDSKVGGNYNYLYRLFRKSNLKPFERDISTEIYQAGSTPCNGVEGFNCYFPLQSYGFQPTNGVGYQPY

SEQ ID NO: 21 - DNA, 29903 bp, SARS-CoV-2
attaaaggtttataccttcccaggtaacaaaccaaccaactttcgatctcttgtagatctgttctctaaacgaactttaaaatctgtgtggctgtcactcggctgcatgcttagtgcactcacgcagtataattaataactaattactgtcgttgacaggacacgagtaactcgtctatcttctgcaggctgcttacggtttcgtccgtgttgcagccgatcatcagcacatctaggtttcgtccgggtgtgaccgaaaggtaagatggagagccttgtccctggtttcaacgagaaaacacacgtccaactcagtttgcctgttttacaggttcgcgacgtgctcgtacgtggctttggagactccgtggaggaggtcttatcagaggcacgtcaacatcttaaagatggcacttgtggcttagtagaagttgaaaaaggcgttttgcctcaacttgaacagccctatgtgttcatcaaacgttcggatgctcgaactgcacctcatggtcatgttatggttgagctggtagcagaactcgaaggcattcagtacggtcgtagtggtgagacacttggtgtccttgtccctcatgtgggcgaaataccagtggcttaccgcaaggttcttcttcgtaagaacggtaataaaggagctggtggccatagttacggcgccgatctaaagtcatttgacttaggcgacgagcttggcactgatccttatgaagattttcaagaaaactggaacactaaacatagcagtggtgttacccgtgaactcatgcgtgagcttaacggaggggcatacactcgctatgtcgataacaacttctgtggccctgatggctaccctcttgagtgcattaaagaccttctagcacgtgctggtaaagcttcatgcactttgtccgaacaactggactttattgacactaagaggggtgtatactgctgccgtgaacatgagcatgaaattgcttggtacacggaacgttctgaaaagagctatgaattgcagacaccttttgaaattaaattggcaaagaaatttgacaccttcaatggggaatgtccaaattttgtatttcccttaaattccataatcaagactattcaaccaagggttgaaaagaaaaagcttgatggctttatgggtagaattcgatctgtctatccagttgcgtcaccaaatgaatgcaaccaaatgtgcctttcaactctcatgaagtgtgatcattgtggtgaaacttcatggcagacgggcgattttgttaaagccacttgcgaattttgtggcactgagaatttgactaaagaaggtgccactacttgtggttacttaccccaaaatgctgttgttaaaatttattgtccagcatgtcacaattcagaagtaggacctgagcatagtcttgccgaataccataatgaatctggcttgaaaaccattcttcgtaagggtggtcgcactattgcctttggaggctgtgtgttctcttatgttggttgccataacaagtgtgcctattgggttccacgtgctagcgctaacataggttgtaaccatacaggtgttgttggagaaggttccgaaggtcttaatgacaaccttcttgaaatactccaaaaagagaaagtcaacatcaatattgttggtgactttaaacttaatgaagagatcgccattattttggcatctttttctgcttccacaagtgcttttgtggaaactgtgaaaggtttggattataaagcattcaaacaaattgttgaatcctgtggtaattttaaagttacaaaaggaaaagctaaaaaaggtgcctggaatattggtgaacagaaatcaatactgagtcctctttatgcatttgcatcagaggctgctcgtgttgtacgatcaattttctcccgcactcttgaaactgctcaaaattctgtgcgtgttttacagaaggccgctataacaatactagatggaatttcacagtattcactgagactcattgatgctatgatgttcacatctgatttggctactaacaatctagttgtaatggcctacattacaggtggtgttgttcagttgacttcgcagtggctaactaacatctttggcactgtttatgaaaaactcaaacccgtccttgattggcttgaagagaagtttaaggaaggtgtagagtttcttagagacggttgggaaattgttaaatttatctcaacctgtgcttgtgaaattgtcggtggacaaattgtcacctgtgcaaaggaaattaaggagagtgttcagacattctttaagcttgtaaataaatttttggctttgtgtgctgactctatcattattggtggagctaaacttaaagccttgaatttaggtgaaacatttgtcacgcactcaaagggattgtacagaaagtgtgttaaatccagagaagaaactggcctactcatgcctctaaaagccccaaaagaaattatcttcttagagggagaaacacttcccacagaagtgttaacagaggaagttgtcttgaaaactggtgatttacaaccattagaacaacctactagtgaagctgttgaagctccattggttggtacaccagtttgtattaacgggcttatgttgctcgaaatcaaagacacagaaaagtactgtgcccttgcacctaatatgatggtaacaaacaataccttcacactcaaaggcggtgcaccaacaaaggttacttttggtgatgacactgtgatagaagtgcaaggttacaagagtgtgaatatcacttttgaacttgatgaaaggattgataaagtacttaatgagaagtgctctgcctatacagttgaactcggtacagaagtaaatgagttcgcctgtgttgtggcagatgctgtcataaaaactttgcaaccagtatctgaattacttacaccactgggcattgatttagatgagtggagtatggctacatactacttatttgatgagtctggtgagtttaaattggcttcacatatgtattgttctttctaccctccagatgaggatgaagaagaaggtgattgtgaagaagaagagtttgagccatcaactcaatatgagtatggtactgaagatgattaccaaggtaaacctttggaatttggtgccacttctgctgctcttcaacctgaagaagagcaagaagaagattggttagatgatgatagtcaacaaactgttggtcaacaagacggcagtgaggacaatcagacaactactattcaaacaattgttgaggttcaacctcaattagagatggaacttacaccagttgttcagactattgaagtgaatagttttagtggttatttaaaacttactgacaatgtatacattaaaaatgcagacattgtggaagaagctaaaaaggtaaaaccaacagtggttgttaatgcagccaatgtttaccttaaacatggaggaggtgttgcaggagccttaaataaggctactaacaatgccatgcaagttgaatctgatgattacatagctactaatggaccacttaaagtgggtggtagttgtgttttaagcggacacaatcttgctaaacactgtcttcatgttgtcggcccaaatgttaacaaaggtgaagacattcaacttcttaagagtgcttatgaaaattttaatcagcacgaagttctacttgcaccattattatcagctggtatttttggtgctgaccctatacattctttaagagtttgtgtagatactgttcgcacaaatgtctacttagctgtctttgataaaaatctctatgacaaacttgtttcaagctttttggaaatgaagagtgaaaagcaagttgaacaaaagatcgctgagattcctaaagaggaagttaagccatttataactgaaagtaaaccttcagttgaacagagaaaacaagatgataagaaaatcaaagcttgtgttgaagaagttacaacaactctggaagaaactaagttcctcacagaaaacttgttactttatattgacattaatggcaatcttcatccagattctgccactcttgttagtgacattgacatcactttcttaaagaaagatgctccatatatagtgggtgatgttgttcaagagggtgttttaactgctgtggttatacctactaaaaaggctggtggcactactgaaatgctagcgaaagctttgagaaaagtgccaacagacaattatataaccacttacccgggtcagggtttaaatggttacactgtagaggaggcaaagacagtgcttaaaaagtgtaaaagtgccttttacattctaccatctattatctctaatgagaagcaagaaattcttggaactgtttcttggaatttgcgagaaatgcttgcacatgcagaagaaacacgcaaattaatgcctgtctgtgtggaaactaaagccatagtttcaactatacagcgtaaatataagggtattaaaatacaagagggtgtggttgattatggtgctagattttacttttacaccagtaaaacaactgtagcgtcacttatcaacacacttaacgatctaaatgaaactcttgttacaatgccacttggctatgtaacacatggcttaaatttggaagaagctgctcggtatatgagatctctcaaagtgccagctacagtttctgtttcttcacctgatgctgttacagcgtataatggttatcttacttcttcttctaaaacacctgaagaacattttattgaaaccatctcacttgctggttcctataaagattggtcctattctggacaatctacacaactaggtatagaatttcttaagagaggtgataaaagtgtatattacactagtaatcctaccacattccacctagatggtgaagttatcacctttgacaatcttaagacacttctttctttgagagaagtgaggactattaaggtgtttacaacagtagacaacattaacctccacacgcaagttgtggacatgtcaatgacatatggacaacagtttggtccaacttatttggatggagctgatgttactaaaataaaacctcataattcacatgaaggtaaaacattttatgttttacctaatgatgacactctacgtgttgaggcttttgagtactaccacacaactgatcctagttttctgggtaggtacatgtcagcattaaatcacactaaaaagtggaaatacccacaagttaatggtttaacttctattaaatgggcagataacaactgttatcttgccactgcattgttaacactccaacaaatagagttgaagtttaatccacctgctctacaagatgcttattacagagcaagggctggtgaagctgctaacttttgtgcacttatcttagcctactgtaataagacagtaggtgagttaggtgatgttagagaaacaatgagttacttgtttcaacatgccaatttagattcttgcaaaagagtcttgaacgtggtgtgtaaaacttgtggacaacagcagacaacccttaagggtgtagaagctgttatgtacatgggcacactttcttatgaacaatttaagaaaggtgttcagataccttgtacgtgtggtaaacaagctacaaaatatctagtacaacaggagtcaccttttgttatgatgtcagcaccacctgctcagtatgaacttaagcatggtacatttacttgtgctagtgagtacactggtaattaccagtgtggtcactataaacatataacttctaaagaaactttgtattgcatagacggtgctttacttacaaagtcctcagaatacaaaggtcctattacggatgttttctacaaagaaaacagttacacaacaaccataaaaccagttacttataaattggatggtgttgtttgtacagaaattgaccctaagttggacaattattataagaaagacaattcttatttcacagagcaaccaattgatcttgtaccaaaccaaccatatccaaacgcaagcttcgataattttaagtttgtatgtgataatatcaaatttgctgatgatttaaaccagttaactggttataagaaacctgcttcaagagagcttaaagttacatttttccctgacttaaatggtgatgtggtggctattgattataaacactacacaccctcttttaagaaaggagctaaattgttacataaacctattgtttggcatgttaacaatgcaactaataaagccacgtataaaccaaatacctggtgtatacgttgtctttggagcacaaaaccagttgaaacatcaaattcgtttgatgtactgaagtcagaggacgcgcagggaatggataatcttgcctgcgaagatctaaaaccagtctctgaagaagtagtggaaaatcctaccatacagaaagacgttcttgagtgtaatgtgaaaactaccgaagttgtaggagacattatacttaaaccagcaaataatagtttaaaaattacagaagaggttggccacacagatctaatggctgcttatgtagacaattctagtcttactattaagaaacctaatgaattatctagagtattaggtttgaaaacccttgctactcatggtttagctgctgttaatagtgtcccttgggatactatagctaattatgctaagccttttcttaacaaagttgttagtacaactactaacatagttacacggtgtttaaaccgtgtttgtactaattatatgccttatttctttactttattgctacaattgtgtacttttactagaagtacaaattctagaattaaagcatctatgccgactactatagcaaagaatactgttaagagtgtcggtaaattttgtctagaggcttcatttaattatttgaagtcacctaatttttctaaactgataaatattataatttggtttttactattaagtgtttgcctaggttctttaatctactcaaccgctgctttaggtgttttaatgtctaatttaggcatgccttcttactgtactggttacagagaaggctatttgaactctactaatgtcactattgcaacctactgtactggttctataccttgtagtgtttgtcttagtggtttagattctttagacacctatccttctttagaaactatacaaattaccatttcatcttttaaatgggatttaactgcttttggcttagttgcagagtggtttttggcatatattcttttcactaggtttttctatgtacttggattggctgcaatcatgcaattgtttttcagctattttgcagtacattttattagtaattcttggcttatgtggttaataattaatcttgtacaaatggccccgatttcagctatggttagaatgtacatcttctttgcatcattttattatgtatggaaaagttatgtgcatgttgtagacggttgtaattcatcaacttgtatgatgtgttacaaacgtaatagagcaacaagagtcgaatgtacaactattgttaatggtgttagaaggtccttttatgtctatgctaatggaggtaaaggcttttgcaaactacacaattggaattgtgttaattgtgatacattctgtgctggtagtacatttattagtgatgaagttgcgagagacttgtcactacagtttaaaagaccaataaatcctactgaccagtcttcttacatcgttgatagtgttacagtgaagaatggttccatccatctttactttgataaagctggtcaaaagacttatgaaagacattctctctctcattttgttaacttagacaacctgagagctaataacactaaaggttcattgcctattaatgttatagtttttgatggtaaatcaaaatgtgaagaatcatctgcaaaatcagcgtctgtttactacagtcagcttatgtgtcaacctatactgttactagatcaggcattagtgtctgatgttggtgatagtgcggaagttgcagttaaaatgtttgatgcttacgttaatacgttttcatcaacttttaacgtaccaatggaaaaactcaaaacactagttgcaactgcagaagctgaacttgcaaagaatgtgtccttagacaatgtcttatctacttttatttcagcagctcggcaagggtttgttgattcagatgtagaaactaaagatgttgttgaatgtcttaaattgtcacatcaatctgacatagaagttactggcgatagttgtaataactatatgctcacctataacaaagttgaaaacatgacaccccgtgaccttggtgcttgtattgactgtagtgcgcgtcatattaatgcgcaggtagcaaaaagtcacaacattgctttgatatggaacgttaaagatttcatgtcattgtctgaacaactacgaaaacaaatacgtagtgctgctaaaaagaataacttaccttttaagttgacatgtgcaactactagacaagttgttaatgttgtaacaacaaagatagcacttaagggtggtaaaattgttaataattggttgaagcagttaattaaagttacacttgtgttcctttttgttgctgctattttctatttaataacacctgttcatgtcatgtctaaacatactgacttttcaagtgaaatcataggatacaaggctattgatggtggtgtcactcgtgacatagcatctacagatacttgttttgctaacaaacatgctgattttgacacatggtttagccagcgtggtggtagttatactaatgacaaagcttgcccattgattgctgcagtcataacaagagaagtgggttttgtcgtgcctggtttgcctggcacgatattacgcacaactaatggtgactttttgcatttcttacctagagtttttagtgcagttggtaacatctgttacacaccatcaaaacttatagagtacactgactttgcaacatcagcttgtgttttggctgctgaatgtacaatttttaaagatgcttctggtaagccagtaccatattgttatgataccaatgtactagaaggttctgttgcttatgaaagtttacgccctgacacacgttatgtgctcatggatggctctattattcaatttcctaacacctaccttgaaggttctgttagagtggtaacaacttttgattctgagtactgtaggcacggcacttgtgaaagatcagaagctggtgtttgtgtatctactagtggtagatgggtacttaacaatgattattacagatctttaccaggagttttctgtggtgtagatgctgtaaatttacttactaatatgtttacaccactaattcaacctattggtgctttggacatatcagcatctatagtagctggtggtattgtagctatcgtagtaacatgccttgcctactattttatgaggtttagaagagcttttggtgaatacagtcatgtagttgcctttaatactttactattccttatgtcattcactgtactctgtttaacaccagtttactcattcttacctggtgtttattctgttatttacttgtacttgacattttatcttactaatgatgtttcttttttagcacatattcagtggatggttatgttcacacctttagtacctttctggataacaattgcttatatcatttgtatttccacaaagcatttctattggttctttagtaattacctaaagagacgtgtagtctttaatggtgtttcctttagtacttttgaagaagctgcgctgtgcacctttttgttaaataaagaaatgtatctaaagttgcgtagtgatgtgctattacctcttacgcaatataatagatacttagctctttataataagtacaagtattttagtggagcaatggatacaactagctacagagaagctgcttgttgtcatctcgcaaaggctctcaatgacttcagtaactcaggttctgatgttctttaccaaccaccacaaacctctatcacctcagctgttttgcagagtggttttagaaaaatggcattcccatctggtaaagttgagggttgtatggtacaagtaacttgtggtacaactacacttaacggtctttggcttgatgacgtagtttactgtccaagacatgtgatctgcacctctgaagacatgcttaaccctaattatgaagatttactcattcgtaagtctaatcataatttcttggtacaggctggtaatgttcaactcagggttattggacattctatgcaaaattgtgtacttaagcttaaggttgatacagccaatcctaagacacctaagtataagtttgttcgcattcaaccaggacagactttttcagtgttagcttgttacaatggttcaccatctggtgtttaccaatgtgctatgaggcccaatttcactattaagggttcattccttaatggttcatgtggtagtgttggttttaacatagattatgactgtgtctctttttgttacatgcaccatatggaattaccaactggagttcatgctggcacagacttagaaggtaacttttatggaccttttgttgacaggcaaacagcacaagcagctggtacggacacaactattacagttaatgttttagcttggttgtacgctgctgttataaatggagacaggtggtttctcaatcgatttaccacaactcttaatgactttaaccttgtggctatgaagtacaattatgaacctctaacacaagaccatgttgacatactaggacctctttctgctcaaactggaattgccgttttagatatgtgtgcttcattaaaagaattactgcaaaatggtatgaatggacgtaccatattgggtagtgctttattagaagatgaatttacaccttttgatgttgttagacaatgctcaggtgttactttccaaagtgcagtgaaaagaacaatcaagggtacacaccactggttgttactcacaattttgacttcacttttagttttagtccagagtactcaatggtctttgttcttttttttgtatgaaaatgcctttttaccttttgctatgggtattattgctatgtctgcttttgcaatgatgtttgtcaaacataagcatgcatttctctgtttgtttttgttaccttctcttgccactgtagcttattttaatatggtctatatgcctgctagttgggtgatgcgtattatgacatggttggatatggttgatactagtttgtctggttttaagctaaaagactgtgttatgtatgcatcagctgtagtgttactaatccttatgacagcaagaactgtgtatgatgatggtgctaggagagtgtggacacttatgaatgtcttgacactcgtttataaagtttattatggtaatgctttagatcaagccatttccatgtgggctcttataatctctgttacttctaactactcaggtgtagttacaactgtcatgtttttggccagaggtattgtttttatgtgtgttgagtattgccctattttcttcataactggtaatacacttcagtgtataatgctagtttattgtttcttaggctatttttgtacttgttactttggcctcttttgtttactcaaccgctactttagactgactcttggtgtttatgattacttagtttctacacaggagtttagatatatgaattcacagggactactcccacccaagaatagcatagatgccttcaaactcaacattaaattgttgggtgttggtggcaaaccttgtatcaaagtagccactgtacagtctaaaatgtcagatgtaaagtgcacatcagtagtcttactctcagttttgcaacaactcagagtagaatcatcatctaaattgtgggctcaatgtgtccagttacacaatgacattctcttagctaaagatactactgaagcctttgaaaaaatggtttcactactttctgttttgctttccatgcagggtgctgtagacataaacaagctttgtgaagaaatgctggacaacagggcaaccttacaagctatagcctcagagtttagttcccttccatcatatgcagcttttgctactgctcaagaagcttatgagcaggctgttgctaatggtgattctgaagttgttcttaaaaagttgaagaagtctttgaatgtggctaaatctgaatttgaccgtgatgcagccatgcaacgtaagttggaaaagatggctgatcaagctatgacccaaatgtataaacaggctagatctgaggacaagagggcaaaagttactagtgctatgcagacaatgcttttcactatgcttagaaagttggataatgatgcactcaacaacattatcaacaatgcaagagatggttgtgttcccttgaacataatacctcttacaacagcagccaaactaatggttgtcataccagactataacacatataaaaatacgtgtgatggtacaacatttacttatgcatcagcattgtgggaaatccaacaggttgtagatgcagatagtaaaattgttcaacttagtgaaattagtatggacaattcacctaatttagcatggcctcttattgtaacagctttaagggccaattctgctgtcaaattacagaataatgagcttagtcctgttgcactacgacagatgtcttgtgctgccggtactacacaaactgcttgcactgatgacaatgcgttagcttactacaacacaacaaagggaggtaggtttgtacttgcactgttatccgatttacaggatttgaaatgggctagattccctaagagtgatggaactggtactatctatacagaactggaaccaccttgtaggtttgttacagacacacctaaaggtcctaaagtgaagtatttatactttattaaaggattaaacaacctaaatagaggtatggtacttggtagtttagctgccacagtacgtctacaagctggtaatgcaacagaagtgcctgccaattcaactgtattatctttctgtgcttttgctgtagatgctgctaaagcttacaaagattatctagctagtgggggacaaccaatcactaattgtgttaagatgttgtgtacacacactggtactggtcaggcaataacagttacaccggaagccaatatggatcaagaatcctttggtggtgcatcgtgttgtctgtactgccgttgccacatagatcatccaaatcctaaaggattttgtgacttaaaaggtaagtatgtacaaatacctacaacttgtgctaatgaccctgtgggttttacacttaaaaacacagtctgtaccgtctgcggtatgtggaaaggttatggctgtagttgtgatcaactccgcgaacccatgcttcagtcagctgatgcacaatcgtttttaaacgggtttgcggtgtaagtgcagcccgtcttacaccgtgcggcacaggcactagtactgatgtcgtatacagggcttttgacatctacaatgataaagtagctggttttgctaaattcctaaaaactaattgttgtcgcttccaagaaaaggacgaagatgacaatttaattgattcttactttgtagttaagagacacactttctctaactaccaacatgaagaaacaatttataatttacttaaggattgtccagctgttgctaaacatgacttctttaagtttagaatagacggtgacatggtaccacatatatcacgtcaacgtcttactaaatacacaatggcagacctcgtctatgctttaaggcattttgatgaaggtaattgtgacacattaaaagaaatacttgtcacatacaattgttgtgatgatgattatttcaataaaaaggactggtatgattttgtagaaaacccagatatattacgcgtatacgccaacttaggtgaacgtgtacgccaagctttgttaaaaacagtacaattctgtgatgccatgcgaaatgctggtattgttggtgtactgacattagataatcaagatctcaatggtaactggtatgatttcggtgatttcatacaaaccacgccaggtagtggagttcctgttgtagattcttattattcattgttaatgcctatattaaccttgaccagggctttaactgcagagtcacatgttgacactgacttaacaaagccttacattaagtgggatttgttaaaatatgacttcacggaagagaggttaaaactctttgaccgttattttaaatattgggatcagacataccacccaaattgtgttaactgtttggatgacagatgcattctgcattgtgcaaactttaatgttttattctctacagtgttcccacctacaagttttggaccactagtgagaaaaatatttgttgatggtgttccatttgtagtttcaactggataccacttcagagagctaggtgttgtacataatcaggatgtaaacttacatagctctagacttagttttaaggaattacttgtgtatgctgctgaccctgctatgcacgctgcttctggtaatctattactagataaacgcactacgtgcttttcagtagctgcacttactaacaatgttgcttttcaaactgtcaaacccggtaattttaacaaagacttctatgactttgctgtgtctaagggtttctttaaggaaggaagttctgttgaattaaaacacttcttctttgctcaggatggtaatgctgctatcagcgattatgactactatcgttataatctaccaacaatgtgtgatatcagacaactactatttgtagttgaagttgttgataagtactttgattgttacgatggtggctgtattaatgctaaccaagtcatcgtcaacaacctagacaaatcagctggttttccatttaataaatggggtaaggctagactttattatgattcaatgagttatgaggatcaagatgcacttttcgcatatacaaaacgtaatgtcatccctactataactcaaatgaatcttaagtatgccattagtgcaaagaatagagctcgcaccgtagctggtgtctctatctgtagtactatgaccaatagacagtttcatcaaaaattattgaaatcaatagccgccactagaggagctactgtagtaattggaacaagcaaattctatggtggttggcacaacatgttaaaaactgtttatagtgatgtagaaaaccctcaccttatgggttgggattatcctaaatgtgatagagccatgcctaacatgcttagaattatggcctcacttgttcttgctcgcaaacatacaacgtgttgtagcttgtcacaccgtttctatagattagctaatgagtgtgctcaagtattgagtgaaatggtcatgtgtggcggttcactatatgttaaaccaggtggaacctcatcaggagatgccacaactgcttatgctaatagtgtttttaacatttgtcaagctgtcacggccaatgttaatgcacttttatctactgatggtaacaaaattgccgataagtatgtccgcaatttacaacacagactttatgagtgtctctatagaaatagagatgttgacacagactttgtgaatgagttttacgcatatttgcgtaaacatttctcaatgatgatactctctgacgatgctgttgtgtgtttcaatagcacttatgcatctcaaggtctagtggctagcataaagaactttaagtcagttctttattatcaaaacaatgtttttatgtctgaagcaaaatgttggactgagactgaccttactaaaggacctcatgaattttgctctcaacatacaatgctagttaaacagggtgatgattatgtgtaccttccttacccagatccatcaagaatcctaggggccggctgttttgtagatgatatcgtaaaaacagatggtacacttatgattgaacggttcgtgtctttagctatagatgcttacccacttactaaacatcctaatcaggagtatgctgatgtctttcatttgtacttacaatacataagaaagctacatgatgagttaacaggacacatgttagacatgtattctgttatgcttactaatgataacacttcaaggtattgggaacctgagttttatgaggctatgtacacaccgcatacagtcttacaggctgttggggcttgtgttctttgcaattcacagacttcattaagatgtggtgcttgcatacgtagaccattcttatgttgtaaatgctgttacgaccatgtcatatcaacatcacataaattagtcttgtctgttaatccgtatgtttgcaatgctccaggttgtgatgtcacagatgtgactcaactttacttaggaggtatgagctattattgtaaatcacataaaccacccattagttttccattgtgtgctaatggacaagtttttggtttatataaaaatacatgtgttggtagcgataatgttactgactttaatgcaattgcaacatgtgactggacaaatgctggtgattacattttagctaacacctgtactgaaagactcaagctttttgcagcagaaacgctcaaagctactgaggagacatttaaactgtcttatggtattgctactgtacgtgaagtgctgtctgacagagaattacatctttcatgggaagttggtaaacctagaccaccacttaaccgaaattatgtctttactggttatcgtgtaactaaaaacagtaaagtacaaataggagagtacacctttgaaaaaggtgactatggtgatgctgttgtttaccgaggtacaacaacttacaaattaaatgttggtgattattttgtgctgacatcacatacagtaatgccattaagtgcacctacactagtgccacaagagcactatgttagaattactggcttatacccaacactcaatatctcagatgagttttctagcaatgttgcaaattatcaaaaggttggtatgcaaaagtattctacactccagggaccacctggtactggtaagagtcattttgctattggcctagctctctactacccttctgctcgcatagtgtatacagcttgctctcatgccgctgttgatgcactatgtgagaaggcattaaaatatttgcctatagataaatgtagtagaattatacctgcacgtgctcgtgtagagtgttttgataaattcaaagtgaattcaacattagaacagtatgtcttttgtactgtaaatgcattgcctgagacgacagcagatatagttgtctttgatgaaatttcaatggccacaaattatgatttgagtgttgtcaatgccagattacgtgctaagcactatgtgtacattggcgaccctgctcaattacctgcaccacgcacattgctaactaagggcacactagaaccagaatatttcaattcagtgtgtagacttatgaaaactataggtccagacatgttcctcggaacttgtcggcgttgtcctgctgaaattgttgacactgtgagtgctttggtttatgataataagcttaaagcacataaagacaaatcagctcaatgctttaaaatgttttataagggtgttatcacgcatgatgtttcatctgcaattaacaggccacaaataggcgtggtaagagaattccttacacgtaaccctgcttggagaaaagctgtctttatttcaccttataattcacagaatgctgtagcctcaaagattttgggactaccaactcaaactgttgattcatcacagggctcagaatatgactatgtcatattcactcaaaccactgaaacagctcactcttgtaatgtaaacagatttaatgttgctattaccagagcaaaagtaggcatactttgcataatgtctgatagagacctttatgacaagttgcaatttacaagtcttgaaattccacgtaggaatgtggcaactttacaagctgaaaatgtaacaggactctttaaagattgtagtaaggtaatcactgggttacatcctacacaggcacctacacacctcagtgttgacactaaattcaaaactgaaggtttatgtgttgacatacctggcatacctaaggacatgacctatagaagactcatctctatgatgggttttaaaatgaattatcaagttaatggttaccctaacatgtttatcacccgcgaagaagctataagacatgtacgtgcatggattggcttcgatgtcgaggggtgtcatgctactagagaagctgttggtaccaatttacctttacagctaggtttttctacaggtgttaacctagttgctgtacctacaggttatgttgatacacctaataatacagatttttccagagttagtgctaaaccaccgcctggagatcaatttaaacacctcataccacttatgtacaaaggacttccttggaatgtagtgcgtataaagattgtacaaatgttaagtgacacacttaaaaatctctctgacagagtcgtatttgtcttatgggcacatggctttgagttgacatctatgaagtattttgtgaaaataggacctgagcgcacctgttgtctatgtgatagacgtgccacatgcttttccactgcttcagacacttatgcctgttggcatcattctattggatttgattacgtctataatccgtttatgattgatgttcaacaatggggttttacaggtaacctacaaagcaaccatgatctgtattgtcaagtccatggtaatgcacatgtagctagttgtgatgcaatcatgactaggtgtctagctgtccacgagtgctttgttaagcgtgttgactggactattgaatatcctataattggtgatgaactgaagattaatgcggcttgtagaaaggttcaacacatggttgttaaagctgcattattagcagacaaattcccagttcttcacgacattggtaaccctaaagctattaagtgtgtacctcaagctgatgtagaatggaagttctatgatgcacagccttgtagtgacaaagcttataaaatagaagaattattctattcttatgccacacattctgacaaattcacagatggtgtatgcctattttggaattgcaatgtcgatagatatcctgctaattccattgtttgtagatttgacactagagtgctatctaaccttaacttgcctggttgtgatggtggcagtttgtatgtaaataaacatgcattccacacaccagcttttgataaaagtgcttttgttaatttaaaacaattaccatttttctattactctgacagtccatgtgagtctcatggaaaacaagtagtgtcagatatagattatgtaccactaaagtctgctacgtgtataacacgttgcaatttaggtggtgctgtctgtagacatcatgctaatgagtacagattgtatctcgatgcttataacatgatgatctcagctggctttagcttgtgggtttacaaacaatttgatacttataacctctggaacacttttacaagacttcagagtttagaaaatgtggcttttaatgttgtaaataagggacactttgatggacaacagggtgaagtaccagtttctatcattaataacactgtttacacaaaagttgatggtgttgatgtagaattgtttgaaaataaaacaacattacctgttaatgtagcatttgagctttgggctaagcgcaacattaaaccagtaccagaggtgaaaatactcaataatttgggtgtggacattgctgctaatactgtgatctgggactacaaaagagatgctccagcacatatatctactattggtgtttgttctatgactgacatagccaagaaaccaactgaaacgatttgtgcaccactcactgtcttttttgatggtagagttgatggtcaagtagacttatttagaaatgcccgtaatggtgttcttattacagaaggtagtgttaaaggtttacaaccatctgtaggtcccaaacaagctagtcttaatggagtcacattaattggagaagccgtaaaaacacagttcaattattataagaaagttgatggtgttgtccaacaattacctgaaacttactttactcagagtagaaatttacaagaatttaaacccaggagtcaaatggaaattgatttcttagaattagctatggatgaattcattgaacggtataaattagaaggctatgccttcgaacatatcgtttatggagattttagtcatagtcagttaggtggtttacatctactgattggactagctaaacgttttaaggaatcaccttttgaattagaagattttattcctatggacagtacagttaaaaactatttcataacagatgcgcaaacaggttcatctaagtgtgtgtgttctgttattgatttattacttgatgattttgttgaaataataaaatcccaagatttatctgtagtttctaaggttgtcaaagtgactattgactatacagaaatttcatttatgctttggtgtaaagatggccatgtagaaacattttacccaaaattacaatctagtcaagcgtggcaaccgggtgttgctatgcctaatctttacaaaatgcaaagaatgctattagaaaagtgtgaccttcaaaattatggtgatagtgcaacattacctaaaggcataatgatgaatgtcgcaaaatatactcaactgtgtcaatatttaaacacattaacattagctgtaccctataatatgagagttatacattttggtgctggttctgataaaggagttgcaccaggtacagctgttttaagacagtggttgcctacgggtacgctgcttgtcgattcagatcttaatgactttgtctctgatgcagattcaactttgattggtgattgtgcaactgtacatacagctaataaatgggatctcattattagtgatatgtacgaccctaagactaaaaatgttacaaaagaaaatgactctaaagagggttttttcacttacatttgtgggtttatacaacaaaagctagctcttggaggttccgtggctataaagataacagaacattcttggaatgctgatctttataagctcatgggacacttcgcatggtggacagcctttgttactaatgtgaatgcgtcatcatctgaagcatttttaattggatgtaattatcttggcaaaccacgcgaacaaatagatggttatgtcatgcatgcaaattacatattttggaggaatacaaatccaattcagttgtcttcctattctttatttgacatgagtaaatttccccttaaattaaggggtactgctgttatgtctttaaaagaaggtcaaatcaatgatatgattttatctcttcttagtaaaggtagacttataattagagaaaacaacagagttgttatttctagtgatgttcttgttaacaactaaacgaacaatgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataaacgaacttatggatttgtttatgagaatcttcacaattggaactgtaactttgaagcaaggtgaaatcaaggatgctactccttcagattttgttcgcgctactgcaacgataccgatacaagcctcactccctttcggatggcttattgttggcgttgcacttcttgctgtttttcagagcgcttccaaaatcataaccctcaaaaagagatggcaactagcactctccaagggtgttcactttgtttgcaacttgctgttgttgtttgtaacagtttactcacaccttttgctcgttgctgctggccttgaagccccttttctctatctttatgctttagtctacttcttgcagagtataaactttgtaagaataataatgaggctttggctttgctggaaatgccgttccaaaaacccattactttatgatgccaactattttctttgctggcatactaattgttacgactattgtataccttacaatagtgtaacttcttcaattgtcattacttcaggtgatggcacaacaagtcctatttctgaacatgactaccagattggtggttatactgaaaaatgggaatctggagtaaaagactgtgttgtattacacagttacttcacttcagactattaccagctgtactcaactcaattgagtacagacactggtgttgaacatgttaccttcttcatctacaataaaattgttgatgagcctgaagaacatgtccaaattcacacaatcgacggttcatccggagttgttaatccagtaatggaaccaatttatgatgaaccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactctcgtgttaaaaatctgaattcttctagagttcctgatcttctggtctaaacgaactaaatattatattagtttttctgtttggaactttaattttagccatggcagattccaacggtactattaccgttgaagagcttaaaaagctccttgaacaatggaacctagtaataggtttcctattccttacatggatttgtcttctacaatttgcctatgccaacaggaataggtttttgtatataattaagttaattttcctctggctgttatggccagtaactttagcttgttttgtgcttgctgctgtttacagaataaattggatcaccggtggaattgctatcgcaatggcttgtcttgtaggcttgatgtggctcagctacttcattgcttctttcagactgtttgcgcgtacgcgttccatgtggtcattcaatccagaaactaacattcttctcaacgtgccactccatggcactattctgaccagaccgcttctagaaagtgaactcgtaatcggagctgtgatccttcgtggacatcttcgtattgctggacaccatctaggacgctgtgacatcaaggacctgcctaaagaaatcactgttgctacatcacgaacgctttcttattacaaattgggagcttcgcagcgtgtagcaggtgactcaggttttgctgcatacagtcgctacaggattggcaactataaattaaacacagaccattccagtagcagtgacaatattgctttgcttgtacagtaagtgacaacagatgtttcatctcgttgactttcaggttactatagcagagatattactaattattatgaggacttttaaagtttccatttggaatcttgattacatcataaacctcataattaaaaatttatctaagtcactaactgagaataaatattctcaattagatgaagagcaaccaatggagattgattaaacgaacatgaaaattattcttttcttggcactgataacactcgctacttgtgagctttatcactaccaagagtgtgttagaggtacaacagtacttttaaaagaaccttgctcttctggaacatacgagggcaattcaccatttcatcctctagctgataacaaatttgcactgacttgctttagcactcaatttgcttttgcttgtcctgacggcgtaaaacacgtctatcagttacgtgccagatcagtttcacctaaactgttcatcagacaagaggaagttcaagaactttactctccaatttttcttattgttgcggcaatagtgtttataacactttgcttcacactcaaaagaaagacagaatgattgaactttcattaattgacttctatttgtgctttttagcctttctgctattccttgttttaattatgcttattatcttttggttctcacttgaactgcaagatcataatgaaacttgtcacgcctaaacgaacatgaaatttcttgttttcttaggaatcatcacaactgtagctgcatttcaccaagaatgtagtttacagtcatgtactcaacatcaaccatatgtagttgatgacccgtgtcctattcacttctattctaaatggtatattagagtaggagctagaaaatcagcacctttaattgaattgtgcgtggatgaggctggttctaaatcacccattcagtacatcgatatcggtaattatacagtttcctgtttaccttttacaattaattgccaggaacctaaattgggtagtcttgtagtgcgttgttcgttctatgaagactttttagagtatcatgacgttcgtgttgttttagatttcatctaaacgaacaaactaaaatgtctgataatggaccccaaaatcagcgaaatgcaccccgcattacgtttggtggaccctcagattcaactggcagtaaccagaatggagaacgcagtggggcgcgatcaaaacaacgtcggccccaaggtttacccaataatactgcgtcttggttcaccgctctcactcaacatggcaaggaagaccttaaattccctcgaggacaaggcgttccaattaacaccaatagcagtccagatgaccaaattggctactaccgaagagctaccagacgaattcgtggtggtgacggtaaaatgaaagatctcagtccaagatggtatttctactacctaggaactgggccagaagctggacttccctatggtgctaacaaagacggcatcatatgggttgcaactgagggagccttgaatacaccaaaagatcacattggcacccgcaatcctgctaacaatgctgcaatcgtgctacaacttcctcaaggaacaacattgccaaaaggcttctacgcagaagggagcagaggcggcagtcaagcctcttctcgttcctcatcacgtagtcgcaacagttcaagaaattcaactccaggcagcagtaggggaacttctcctgctagaatggctggcaatggcggtgatgctgctcttgctttgctgctgcttgacagattgaaccagcttgagagcaaaatgtctggtaaaggccaacaacaacaaggccaaactgtcactaagaaatctgctgctgaggcttctaagaagcctcggcaaaaacgtactgccactaaagcatacaatgtaacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttcaaagatcaagtcattttgctgaataagcatattgacgcatacaaaacattcccaccaacagagcctaaaaaggacaaaaagaagaaggctgatgaaactcaagccttaccgcagagacagaagaaacagcaaactgtgactcttcttcctgctgcagatttggatgatttctccaaacaattgcaacaatccatgagcagtgctgactcaactcaggcctaaactcatgcagaccacacaaggcagatgggctatataaacgttttcgcttttccgtttacgatatatagtctactcttgtgcagaatgaattctcgtaactacatagcacaagtagatgtagttaactttaatctcacatagcaatctttaatcagtgtgtaacattagggaggacttgaaagagccaccacattttcaccgaggccacgcggagtacgatcgagtgtacagtgaacaatgctagggagagctgcctatatggaagagccctaatgtgtaaaattaattttagtagtgctatccccatgtgattttaatagcttcttaggagaatgacaaaaaaaaaaaaaaaaaaaaaaaaaaaaaaaaa

SEQ ID NO: 22 - protein, 8 aa, SARS-CoV-2
DRVYIHPF

SEQ ID NO: 23 - protein, 15 aa, SARS-CoV-2
RTQLPPAYTNSFTRG

SEQ ID NO: 24 - protein, 15 aa, SARS-CoV-2
PAYTNSFTRGVYYPD

SEQ ID NO: 25 - protein, 15 aa, SARS-CoV-2
GGNYNYLYRLFRKSN

SEQ ID NO: 26 - protein, 15 aa, SARS-CoV-2
VLTESNKKFLPFQQF

SEQ ID NO: 27 - protein, 15 aa, SARS-CoV-2
NKKFLPFQQFGRDIA

SEQ ID NO: 28 - protein, 15 aa, SARS-CoV-2
PFQQFGRDIADTTDA

SEQ ID NO: 29 - protein, 15 aa, SARS-CoV-2
GRDIADTTDAVRDPQ

SEQ ID NO: 30 - protein, 15 aa, SARS-CoV-2
DTTDAVRDPQTLEIL

SEQ ID NO: 31 - protein, 15 aa, SARS-CoV-2
ECDIPIGAGICASYQ

SEQ ID NO: 32 - protein, 15 aa, SARS-CoV-2
GIVNNTVYDPLQPEL

SEQ ID NO: 33 - protein, 15 aa, SARS-CoV-2
TVYDPLQPELDSFKE

SEQ ID NO: 34 - protein, 15 aa, SARS-CoV-2
DSFKEELDKYFKNHT

SEQ ID NO: 35 - protein, 15 aa, SARS-CoV-2
VVNIQKEIDRLNEVA

SEQ ID NO: 36 - protein, 20 aa, SARS-CoV-2
RTQLPPAYTNSFTRGVYYPD

SEQ ID NO: 37 - protein, 22 aa, SARS-CoV-2
GNYNYLYRLFRKSNLKPFERDI

SEQ ID NO: 38 - protein, 35 aa, SARS-CoV-2
VLTESNKKFLPFQQFGRDIADTTDAVRDPQTLEIL

SEQ ID NO: 39 - protein, 19 aa, SARS-CoV-2
KQIYKTPPIKDFGGFNFSQ

SEQ ID NO: 40 - protein, 31 aa, SARS-CoV-2
GIVNNTVYDPLQPELDSFKEELPDKYFKNHT

(19) United States
(12) Patent Application Publication
(10) Pub. No.: US 2024/0044896 A1
(43) Pub. Date: Feb. 8, 2024

(54) METHODS AND SYSTEMS FOR DETECTION AND ANALYSIS OF ANGIOTENSIN BINDING ANTIBODIES

(71) Applicant: The University of Chicago, Chicago, IL (US)

(72) Inventors: Melody SWARTZ, Chicago, IL (US); Priscilla BRIQUEZ, Chicago, IL (US); Jeffrey HUBBELL, Chicago, IL (US)

(73) Assignee: The University of Chicago, Chicago, IL (US)

(21) Appl. No.: 18/266,381

(22) PCT Filed: Dec. 9, 2021
(86) PCT No.: PCT/US21/72823

§ 371 Date: Jun. 9, 2023

Related U.S. Application Data
(60) Provisional application No. 63/123,199, filed on Dec. 9, 2020.

(51) Int. Cl. G01N 33/569; C07K 14/165

(52) U.S. Cl. CPC G01N 33/56983; C07K 14/165; G01N 2333/165; G01N 2333/575; G01N 2469/20; G01N 2800/32

(57) ABSTRACT

Aspects of the disclosure are directed to methods, systems, and compositions for detecting antibodies capable of binding to angiotensin II. Certain aspects comprise detection of antibodies capable of binding to angiotensin II in a sample from a subject, where the subject has or has had a coronavirus infection, such as a SARS-CoV-2 infection. Also disclosed are vaccine compositions comprising a portion of SARS-CoV-2 Spike protein, where such compositions do not induce generation of angiotensin II-binding antibodies.

### CROSS-REFERENCE TO RELATED APPLICATIONS

This application claims the benefit of priority of U.S. Provisional Patent Application No. 63/123,199 filed Dec. 9, 2020, which is hereby incorporated by reference in its entirety.

### BACKGROUND

### I. Field of the Invention

Aspects of this invention relate to at least the fields of immunology, virology, and medicine.

### II. Background

The virus SARS-CoV-2 recognizes its binding site on target cells, the cell-surface protein angiotensin-converting enzyme-2 (ACE2), through the receptor-binding motif (RMB) of the receptor-binding domain (RBD) of the viral surface protein Spike. Natural infection with SARS-CoV-2 induces anti-Spike and anti-RBD antibodies that can prevent continued and potentially repeated infection. Vaccine forms used to prevent infection with SARS-CoV-2 typically comprise Spike or RBD, either as recombinant proteins, as components of inactivated or attenuated virus, or genetically encoded in vaccinal viruses, mRNA, or DNA. The response to vaccination also induces anti-Spike and/or anti-RBD antibodies, depending on the components of the vaccine formulation.

### SUMMARY

Aspects of the disclosure are directed to methods of evaluating a subject having had a SARS-CoV-2 infection, or having received a SARS-CoV-2 vaccine, comprising detecting, in a sample from the subject, an antibody that binds to angiotensin II. Also disclosed are vaccine compositions comprising at least portion of SARS-CoV-2 Spike protein, or a nucleic acid encoding for at least portion of SARS-CoV-2 Spike protein, where the vaccine composition does not induce generation of antibodies that bind to angiotensin II when administered to a subject.

Embodiments of the disclosure include detection methods, diagnosis methods, prognosis methods, methods for evaluating a subject, methods for diagnosing a subject, methods for treating a subject, methods for preventing a coronavirus infection, methods for detecting antibodies that bind to AngII, methods for detecting antibodies that bind to SARS-CoV-2 Spike protein, vaccine compositions, pharmaceutical compositions, excipients, adjuvants, polypeptides, antibodies, aptamers, and nucleic acids.

Compositions of the disclosure can include at least 1, 2, 3, 4, or more of the following components: SARS-CoV-2 Spike protein, a nucleic acid encoding for SARS-CoV-2 Spike protein, a portion of a SARS-CoV-2 Spike protein, a nucleic acid encoding for a portion of a SARS-CoV-2 Spike protein, a polypeptide, an antibody, an excipient, or an adjuvant. It is specifically contemplated that one or more of these components may be excluded from embodiments of the disclosure.

Methods of the disclosure can include at least 1, 2, 3, 4, or more of the following steps: evaluating a subject, diagnosing a subject, treating a subject, obtaining a biological sample, obtaining a blood sample, obtaining a plasma sample, obtaining a serum sample, vaccinating a subject, administering a vaccine to a subject, administering a SARS-CoV-2 therapeutic to a subject, detecting an antibody that binds to angiotensin II, detecting an antibody that binds to SARS-CoV-2 Spike protein, performing an enzyme-linked immunosorbent assay (ELISA), and performing a lateral flow assay. It is specifically contemplated that one or more of these steps may be excluded from embodiments of the disclosure.

Disclosed herein, in some aspects, is a method for evaluating a subject having or having had a coronavirus infection, the method comprising detecting, in a biological sample from the subject, an antibody that binds to angiotensin II. In some aspects, disclosed herein is method for evaluating a subject having been administered a coronavirus vaccine, the method comprising detecting, in a biological sample from the subject, an antibody that binds to angiotensin II.

In some embodiments, the coronavirus is SARS-CoV-2. In some embodiments, the antibody further binds to SARS-CoV-2 Spike protein. In some embodiments, the antibody binds to a region of SARS-CoV-2 Spike protein comprising one of SEQ ID NOs: 1-16 or 22-40. In some embodiments, the antibody binds to a region of SARS-CoV-2 Spike protein comprising SEQ ID NO:1. In some embodiments, the method further comprises obtaining the biological sample from the subject. In some embodiments, the biological sample is a blood sample. In some embodiments, the biological sample is a plasma sample. In some embodiments, the biological sample is a serum sample. In some embodiments, detecting the antibody comprises an ELISA. In some embodiments, detecting the antibody comprises a lateral flow assay. In some embodiments, detecting the antibody comprises a surface plasmon resonance assay.

Disclosed herein, in some aspects, is a method for treating or preventing a SARS-CoV-2 infection comprising administering to a subject a composition comprising (i) a portion of SARS-CoV-2 Spike protein or (ii) a nucleic acid encoding for a portion of SARS-CoV-2 Spike protein. In some embodiments, the composition further comprises a pharmaceutically acceptable excipient. In some embodiments, the subject develops antibodies that bind to SARS-CoV-2 Spike protein. In some embodiments, the subject does not develop antibodies that bind to angiotensin II. The methods may comprise or further comprise evaluating the subject for the presence of angiotensin II autoantibodies in a biological sample from the subject. Further disclosed, in some aspects, is a pharmaceutical composition comprising (a) (i) a portion of a SARS-CoV-2 Spike protein or (ii) a nucleic acid encoding for a portion of a SARS-CoV-2 Spike protein; and (b) a pharmaceutically acceptable excipient. In some embodiments, the pharmaceutical composition further comprises an adjuvant. In some embodiments, the pharmaceutical composition is a vaccine composition. In some embodiments, the pharmaceutical composition is useful for treating or preventing COVID-19.

In some embodiments, the portion of SARS-CoV-2 Spike protein does not comprise SEQ ID NO:1. In some embodiments, the portion of SARS-CoV-2 Spike protein does not comprise SEQ ID NO:2. In some embodiments, the portion of SARS-CoV-2 Spike protein does not comprise SEQ ID NO:3. In some embodiments, the portion of SARS-CoV-2 Spike protein does not comprise SEQ ID NO:4. In some embodiments, the portion of SARS-CoV-2 Spike protein does not comprise SEQ ID NO:5. In some embodiments, the portion of SARS-CoV-2 Spike protein does not comprise SEQ ID NO:6. In some embodiments, the portion of SARS-CoV-2 Spike protein does not comprise SEQ ID NO:7. In some embodiments, the portion of SARS-CoV-2 Spike protein does not comprise SEQ ID NO:8. In some embodiments, the portion of SARS-CoV-2 Spike protein does not comprise SEQ ID NO:9. In some embodiments, the portion of SARS-CoV-2 Spike protein does not comprise SEQ ID NO:10. In some embodiments, the portion of SARS-CoV-2 Spike protein does not comprise SEQ ID NO:11. In some embodiments, the portion of SARS-CoV-2 Spike protein does not comprise SEQ ID NO:12. In some embodiments, the portion of SARS-CoV-2 Spike protein does not comprise SEQ ID NO:13. In some embodiments, the portion of SARS-CoV-2 Spike protein does not comprise SEQ ID NO:14. In some embodiments, the portion of SARS-CoV-2 Spike protein does not comprise SEQ ID NO:15. In some embodiments, the portion of SARS-CoV-2 Spike protein does not comprise SEQ ID NO:16. In some embodiments, the portion of SARS-CoV-2 Spike protein does not comprise SEQ ID NO:22. In some embodiments, the portion of SARS-CoV-2 Spike protein does not comprise SEQ ID NO:23. In some embodiments, the portion of SARS-CoV-2 Spike protein does not comprise SEQ ID NO:24. In some embodiments, the portion of SARS-CoV-2 Spike protein does not comprise SEQ ID NO:25. In some embodiments, the portion of SARS-CoV-2 Spike protein does not comprise SEQ ID NO:26. In some embodiments, the portion of SARS-CoV-2 Spike protein does not comprise SEQ ID NO:27. In some embodiments, the portion of SARS-CoV-2 Spike protein does not comprise SEQ ID NO:28. In some embodiments, the portion of SARS-CoV-2 Spike protein does not comprise SEQ ID NO:29. In some embodiments, the portion of SARS-CoV-2 Spike protein does not comprise SEQ ID NO:30. In some embodiments, the portion of SARS-CoV-2 Spike protein does not comprise SEQ ID NO:31. In some embodiments, the portion of SARS-CoV-2 Spike protein does not comprise SEQ ID NO:32. In some embodiments, the portion of SARS-CoV-2 Spike protein does not comprise SEQ ID NO:33. In some embodiments, the portion of SARS-CoV-2 Spike protein does not comprise SEQ ID NO:34. In some embodiments, the portion of SARS-CoV-2 Spike protein does not comprise SEQ ID NO:35. In some embodiments, the portion of SARS-CoV-2 Spike protein does not comprise SEQ ID NO:36. In some embodiments, the portion of SARS-CoV-2 Spike protein does not comprise SEQ ID NO:37. In some embodiments, the portion of SARS-CoV-2 Spike protein does not comprise SEQ ID NO:38. In some embodiments, the portion of SARS-CoV-2 Spike protein does not comprise SEQ ID NO:39. In some embodiments, the portion of SARS-CoV-2 Spike protein does not comprise SEQ ID NO:40. In some embodiments, the portion of SARS-CoV-2 Spike protein does not comprise any of SEQ ID NOs:1-16 or 22-40. Aspects of the disclosure also relate to compositions comprising polypeptides and nucleic acids encoding polypeptides comprising a portion of the SARS-CoV-2 Spike protein, wherein the polypeptide does not comprise or does not comprise a at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35 of SEQ ID NOS:1-16 or 22-40 or a region with at least 80, 81, 82, 83, 84, 85, 86, 87, 88, 89, 90, 91, 92, 93, 94, 95, 96, 97, 98, 99, or 100% sequence identity to one of SEQ ID NOS:1-16 or 22-40. In some aspects, the polypeptide does not comprise or the nucleic acid encodes for a polypeptide that lacks a region with at least 80, 81, 82, 83, 84, 85, 86, 87, 88, 89, 90, 91, 92, 93, 94, 95, 96, 97, 98, 99, or 100% sequence identity to SEQ ID NO:1, SEQ ID NO:2, SEQ ID NO:3, SEQ ID NO:4, SEQ ID NO:5, SEQ ID NO:6, SEQ ID NO:7, SEQ ID NO:8, to SEQ ID NO:9, SEQ ID NO:10, SEQ ID NO:11, SEQ ID NO:12, SEQ ID NO:13, SEQ ID NO:14, SEQ ID NO:15, SEQ ID NO:16, to SEQ ID NO:22, SEQ ID NO:23, SEQ ID NO:24, SEQ ID NO:25, SEQ ID NO:26, SEQ ID NO:27, SEQ ID NO:28, SEQ ID NO:29, to SEQ ID NO:30, SEQ ID NO:31, SEQ ID NO:32, SEQ ID NO:33, SEQ ID NO:34, SEQ ID NO:35, SEQ ID NO:36, SEQ ID NO:37, to SEQ ID NO:38, SEQ ID NO:39, SEQ ID NO:40, and any derivable combination thereof.

In some aspects, disclosed herein is a method for treating a subject for a SARS-CoV-2 infection comprising administering to the subject an effective amount of a SARS-CoV-2 treatment or a blood pressure regulating agent, wherein a biological sample from the subject was determined to comprise an antibody that binds to angiotensin II. In some embodiments, the SARS-CoV-2 treatment comprises an anti-SARS-CoV-2 antibody. In some embodiments, the SARS-CoV-2 treatment comprises COVID-19 convalescent plasma. In some embodiments, the SARS-CoV-2 treatment comprises an anti-viral agent. In some embodiments, the biological sample is a blood sample. In some embodiments, the biological sample is a plasma sample. In some embodiments, the biological sample is a serum sample. In some embodiments, the subject has and/or has been diagnosed with hypertension. In some embodiments, the subject previously had hypertension, such as within a time frame of 1, 2, 3, 4, 5, or 6 years. In some aspects, the subject has been diagnosed with hypotension. In some aspects, the subject has been diagnosed with acute hypotension. Methods of the disclosure may comprise or further comprise treating a patient determined to have anti-angiotensin II autoantibodies by hospitalization and/or monitoring of their blood pressure.

The blood pressure regulating agent may comprise a vasopressor. In some aspects, the vasopressor comprises epinephrine, droxidopa, isoproterenol, norepinephrine, phenylephrine, ephedrine, angiotensin II, dobutamine, or combinations thereof. In some aspects, the blood pressure regulating agent comprises a diuretic, angiotensin-converting enzyme (ACE) inhibitor, angiotensin II receptor blocker (ARB), calcium channel blocker, or combinations thereof.

Also disclosed herein, in some aspects, is a method for evaluating a subject comprising detecting, in a biological sample from the subject, an antibody that binds to angiotensin II. In some embodiments, the subject has or has had a coronavirus infection. In some embodiments, the subject has or has had a SARS-CoV-2 infection. In some embodiments, the subject has a SARS-CoV-2 infection. In some embodiments, the method further comprises administering a SARS-CoV-2 treatment or blood pressure regulating agent to the subject. In some embodiments, the SARS-CoV-2 treatment comprises an anti-SARS-CoV-2 antibody. In some embodiments, the SARS-CoV-2 treatment comprises COVID-19 convalescent plasma. In some embodiments, the SARS-CoV-2 treatment comprises an anti-viral agent. In some embodiments, the subject has had a SARS-CoV-2 infection. In some embodiments, the biological sample is a blood sample. In some embodiments, the biological sample is a plasma sample. In some embodiments, the biological sample is a serum sample. In some embodiments, the biological sample is a convalescent plasma sample. In some embodiments, the subject has been administered a SARS-CoV vaccine. In some embodiments, the subject has been administered a SARS-CoV-2 vaccine. In some embodiments, the subject has or has had a cardiovascular disorder. In some embodiments, the cardiovascular disorder is high blood pressure or heart failure. In some embodiments, the antibody further binds to SARS-CoV-2 Spike protein. In some embodiments, the antibody binds to a region of SARS-CoV-2 Spike protein comprising one of SEQ ID NOs: 1-16 or 22-40, or a portion thereof. In some embodiments, the antibody binds to a polypeptide comprising one of SEQ ID NOs: 1-16 or 22-40. In some embodiments, the antibody binds to a region of SARS-CoV-2 Spike protein comprising SEQ ID NO:1. In some embodiments, the antibody binds to a polypeptide comprising SEQ ID NO:1. In some embodiments, the method further comprises obtaining the biological sample from the subject. In some embodiments, the biological sample is a plasma sample. In some embodiments, the biological sample is a serum sample. In some embodiments, detecting the antibody comprises an ELISA. In some embodiments, detecting the antibody comprises a lateral flow assay. In some embodiments, detecting the antibody comprises a surface plasmon resonance assay.

Further disclosed, in some embodiments, is a method for screening a SARS-CoV-2 therapeutic composition comprising detecting, in the therapeutic composition, an antibody that binds to angiotensin II. In some embodiments, the SARS-CoV-2 therapeutic composition comprises COVID-19 convalescent plasma. In some embodiments, the SARS-CoV-2 therapeutic composition comprises an anti-SARS-CoV-2 Spike protein antibody. The anti-angiotensin II antibody in the methods of the disclosure may further be characterized as an anti-angiotensin II autoantibody.

Because the SARS-COV-2 virus causes COVID-19, any embodiment discussed in the context of SARS-COV-2 can be implemented with respect to COVID-19. As used herein, “COVID-19 infection” and “SARS-CoV-2 infection” are used interchangeably and refer to an infection of a subject with a SARS-CoV-2 virus.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention. As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that embodiments described herein in the context of the term “comprising” may also be implemented in the context of the term “consisting of” or “consisting essentially of.”

“Individual, “subject,” and “patient” are used interchangeably and can refer to a human or non-human.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Summary of Invention, Detailed Description, Claims, and Brief Description of the Drawings.

A variety of embodiments are discussed throughout this application. Any embodiment discussed with respect to one aspect applies to other aspects as well and vice versa. Each embodiment described herein is understood to be embodiments that are applicable to all aspects. It is contemplated that any embodiment discussed herein can be implemented with respect to any method or composition, and vice versa. Furthermore, compositions and kits can be used to achieve methods disclosed herein.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 shows a schematic illustrating a model of an autoimmune reaction against AngII upon infection or vaccination by SARS-CoV-2.

FIGS. 2A-2F show detection of specific anti-AngII IgG auto-antibodies as well as anti-RBD IgG in the serum/plasma of COVID-19 patients or convalescent humans. FIG. 2A shows anti-RBD IgG levels and confirms that the COVID patients developed anti-RBD in response to SARS-CoV-2 infection (Mann-Whitney test, p<0.0001). FIGS. 2B and 2B show anti-AngII IgG levels and demonstrate that a subpopulation of patients also developed anti-AngII auto-antibodies in addition to anti-RBD antibodies (FIG. 2B; Z test for proportion, p<0.0001) and that the overall amount levels of anti-AngII antibodies were higher in the COVID patient cohort than in healthy donors or hypertensive non-COVID donors (FIG. 2C, Kruskal-Wallis with Dunn's post-test, p<0.0001). FIG. 2D illustrates the correlation between anti-AngII and anti-RBD in the anti-AngII positive COVID patients (Spearman correlation, R=0.295, p=0.0172). FIGS. 2E and 2F show results from analysis of the levels of anti-RBD (FIG. 2E) and anti-AngII (FIG. 2F) over time, indicating that these antibodies significantly increase between 1-10 and 11-20 days in the subpopulation of anti-AngII positive COVID patients (Wilcoxon matched-pairs signed rank test, p<0.0001 for panel E, p=0.0173 for panel F).

FIGS. 3A and 3B show the increase of anti-AngII autoantibodies in mice upon vaccination with adjuvanted SARS-CoV-2 antigens, namely Spike and RBD. FIG. 3A shows anti-AngII levels generated by each vaccination condition (Z test for proportions, p<0.021). FIG. 3B shows changes in anti-AngII antibody levels during infection, demonstrating a significant increase from week 2 and 4 upon prime and boost vaccinations (repeated measures one-way ANOVA, p=0.016).

FIGS. 4A-4D show cross-binding of a monoclonal mouse anti-AngII clone E7 to SARS-CoV-2 Spike and RBD antigens, as well as its modulation of AngII binding to AT1 receptor. FIG. 4A shows binding of anti-AngII to Spike, RBD or BSA (as a control for nonspecific binding). Anti-AngII binds to both Spike and RBD, yet with a higher affinity to Spike. FIG. 4B shows mapping of the SARS-CoV-2 Spike linear epitopes targeted by anti-AngII clone E7 or by sera of vaccinated mice (pooled serums from 5 mice). FIG. 4C shows peptidic sequences of the linear epitopes of Spike targeted by the monoclonal anti-AngII clone E7 antibody detected in panel B. The peptides of FIG. 4C correspond to SEQ ID NOS: according to the following chart:

| Peptide | SEQ ID NO |
|---|---|
| D7 | 2 |
| D14 | 3 |
| D20 | 4 |
| D22 | 5 |
| D23 | 6 |
| E16 | 7 |
| E17 | 8 |
| F13 | 9 |
| F21 | 10 |
| F22 | 11 |
| G14 | 12 |
| G15 | 13 |
| I4 | 14 |
| I9 | 15 |
| J13 | 16 |
| J14 | 1 |
| Ang II sequence | 22 |

FIG. 4D shows inhibition of FAM-AngII binding to its cognate receptor AT-1 in the presence of the Spike/RBD cross-reactive anti-AngII E7 monoclonal antibody (One-way ANOVA with Tukey's post-test, p<0.0001).

FIGS. 5A-5F show explicit relations between the presence/levels of anti-AngII autoantibodies in COVID-19 patients and the corresponding disease severity, via pulse oximetric measurement of daily median SF ratio, or the ratio of peripheral oxygen saturation (SpO₂) to the fraction of inspired oxygen (FiO₂). SF ratio inversely corresponds to clinical severity, where SF>314 defines low severity, 314>SF>235 defines moderate severity, and SF<235 defines high severity. FIG. 5A shows that anti-AngII(+) COVID-19 patients had, on average, significantly lower SF ratios compared to anti-AngII(−) patients (Mann-Whitney test, p=0.006). FIG. 5B shows that higher percentages of anti-AngII(+) COVID-19 patients had moderate or severe disease compared to those percentages in anti-AngII(−) patients (Chi-squared test, χ²=7.4, p=0.0235). Reciprocally, COVID-19 patients with moderate-high disease severity had significantly higher levels of anti-AngII autoantibodies (FIG. 5C, Mann-Whitney test, p=0.04), and higher fractions of severe and moderate COVID patients were anti-AngII(+) than those with low severity (FIG. 5D, p=0.004 for low severity group by Z test). FIG. 5E-H show that the correlations between anti-AngII antibody levels and disease severity were more pronounced in the subset of patients with pre-existing hypertension (HTN) than in non-HTN patients. FIG. 5E shows that the lower average SF ratios in anti-AngII(+) vs. anti-AngII(−) patients was significant in HTN, but not in non-HTN, COVID-19 patient subsets (Mann-Whitney test, p=0.01 for HTN and p=0.36 for non-HTN). FIG. 5F shows that in HTN patients, the percentages of anti-AngII(+) patients with moderate or severe disease were far greater than those of Anti-AngII(−) patients (Chi-squared test, χ²=6.6, p=0.04), but that these differences were less pronounced and not statistically significant in non-HTN patients (χ²=6.6, p=0.56). Reciprocally, in HTN COVID-19 patients, those with moderate to high severity had increased levels of anti-AngII antibodies (FIG. 5G, Mann-Whitney test, p=0.03), an effect that was not statistically significant in non-HTN COVID-19 patients (p=0.65). Similarly, FIG. 5H highlights that the differences seen in FIG. 5D were more pronounced in the HTN subset of COVID patients, and less pronounced in the non-HTN subset: namely, that the fractions of severe and moderate HTN patients that are AngII(+) were much higher than those fractions of non-HTN patients. This effect was not detectable in non-HTN COVID patients.

FIGS. 6A-6H. SARS-CoV-2 infection induces anti-AngII autoantibodies. The plasma of SARS-CoV-2 convalescent hospitalized patients (COVID, N=115) was analyzed for the presence of anti-AngII and anti-RBD by ELISA. Among them, 63% had with pre-existing hypertension (HTN COVID, N=73). Results were compared to non-COVID hypertensive donors (Non-COVID HTN, N=48) or control donors (N=58). Anti-AngII levels are displayed as the signal absorbance measured in plasma diluted at 1:100. Anti-RBD titers are displayed as log₁₀ values. (Hi=high; Lo=low; (−)=negative; gray thresholds=limits for anti-AngII or anti-RBD positivity) (A) Levels of anti-AngII antibodies in the plasma of COVID patients as compared to non-COVID HTN donors and control donors (median±IQR, Kruskal-Wallis test with Dunn's post-test). An absorbance ≥0.077 and ≥0.154 indicates the limit for anti-AngII positivity and high levels, respectively. (B) Proportion of non-COVID HTN and COVID donors that have high (Hi), low (Lo) or negative (−) levels of anti-AngII (χ² test). (C) Levels of anti-AngII in COVID patients with or without pre-existing HTN (Mann-Whitney test). (D) Proportion of non-HTN COVID and HTN COVID patients that have high (Hi), low (Lo) or negative (−) levels of anti-AngII (χ² test). (E) Proportion of patients with high, low or no titers of anti-RBD among patients having high, low or negative levels of anti-AngII (χ² test). (F) Proportion of patients with high, low or negative levels of anti-AngII among patients having high, low or no titers of anti-RBD (χ² test). (G) Correlation between anti-AngII levels and anti-RBD titers in COVID patients. Patients for which the plasma was analyzed at or after 7 days post-symptoms (DPSO) are indicated in black, whereas those analyzed before 7 DPSO are in grey (Spearman correlation on datapoints ≥7 DPSO). (H) Co-variations of anti-AngII and anti-RBD levels between 1-10 DPSO (tail of the arrow) and 11-20 DPSO (tip of the arrow) matched per patient (arrow, N=25).

FIGS. 7A and 7B. Development of anti-AngII autoantibodies in a subset of mice vaccinated with adjuvanted SARS-CoV-2 antigens. Mice were vaccinated at week 0 and 3 with Spike or RBD proteins adjuvanted with MPLA/alum, CpG-B, AddaS03 or with no adjuvant, or with MPLA/alum only (in absence of SARS-CoV-2 antigen). N=10-25 mice per group. (A) Anti-AngII levels in vaccinated mice plasma at week 4. Significant proportions of mice vaccinated with RBD+MPLA/alum or Spike+MPLA/alum raised autoantibodies against AngII (Z-test for proportions: RBD+MPLA/alum vs MPLA/alum: Z=2.35, Spike+MPLA/alum vs. MPLA/alum: Z=2.45, grey threshold=limit for anti-AngII positivity). (B) Levels of anti-AngII autoantibodies over time (at week 1, 2, 3 and 4) in the mice that were found positive for anti-AngII at week 4 (Friedman test with Dunn's post-test).

FIGS. 8A-8H. Anti-AngII autoantibodies correlates with dysregulated blood pressure (BP) and reduced pulse oxymetric saturation SpO₂/FiO₂ (SF ratio) in hospitalized COVID patients (N=115). Grey threshold indicates positivity of anti-AngII. (A) Anti-AngII levels in the plasma of COVID patients with normal or dysregulated blood pressure (Mann-Whitney test). (B) Levels of anti-AngII antibodies in COVID patients with normal BP as compared to patients with dysregulated BP, categorized as being under vasopressive drugs, exhibiting a large mean arterial pressure variability (large ΔMAP) or having experienced severe acute hypotensive episodes despite their known pre-existing HTN condition (these categories are non-exclusive; Kruskal-Wallis test with Dunn's post-test comparison to normal BP). (C) Proportion of patients with normal or dyregulated BP that have negative (−), low (Lo) or high (Hi) levels of anti-AngII antibodies (χ² test). (D) Proportions of patients with negative, low or high levels of anti-AngII in the COVID patients categories described in panel B. (E) Proportions of patients with negative, low or high levels of anti-AngII in COVID patients with pre-existing HTN and/or dysregulated BP. (F) SF ratio of COVID patients that are negative (−) or positive (+) for anti-AngII autoantibodies (Mann-Whitney test). SF ratio values are used to define disease severity as being mild, moderate or severe. (G) Levels of anti-AngII antibodies in patients suffering from mild, moderate or severe form of COVID, as defined based on SF ratio values (Kruskal-Wallis test with Dunn's post-test). (H) Proportion of patients with negative (−), low (Lo) or high (Hi) levels of anti-AngII antibodies in function to the severity of the disease (χ² test).

FIGS. 9A-9F. Cross-binding of anti-AngII antibodies to the SARS-CoV-2 Spike and RBD antigens. (A) Binding of two commercial monoclonal anti-AngII (clone E7 and clone B938M) to recombinant Spike and RBD at various concentrations by ELISA. (B) Inhibition of AngII binding to its cognate receptor AT1 in presence of the anti-AngII (clone E7 and B938M) on CHO-AT1 cells, measured by flow cytometry (ANOVA test with Tukey's post-test). (C) Binding of a library of monoclonal anti-RBD antibodies against AngII by ELISA (9 out of 36 tested showed signals of low binding). (D) Binding of 3 selected monoclonal anti-RBD (S24-902, S564-265 and S24-1002) to AngII at various concentrations as compared to a non-AngII-binding anti-RBD (S564-14). (E) Binding of monoclonal anti-AngII (clone E7 or B938M) to Spike linear epitopes using a peptide array. Highlighted colored regions represents the most immunogenic and recurrent B cell epitopes of Spike found in COVID patients (Shrock et al. (12), Li et al. (13)) and the associated numbers indicate the amino-acid positions that delineate these regions. The x-axis represents the full-length of Spike (SP=signal peptide; NTD=N-terminal domain; RBD=receptor-binding domain; RBM=receptor-binding motif, CTD=C-terminal domain; NTD, RBD and CTD together constitutes the S1 domain of Spike; T=transmembrane domain; C=cytoplasmic tail). (F) Visualization of the main domains targeted by the monoclonal anti-AngII antibodies on the 3D structure of Spike (PDB=6VXX, (14)). Domains are highlighted in colors on one monomer of the trimeric Spike structure (RBD is in black; aa #=amino acid position of Spike).

FIGS. 10A-10D. Anti-AngII does not correlate with sex, age and body mass index (BMI) in hospitalized COVID patients (N=112). Grey threshold=limit for anti-AngII positivity. (A) Correlation between the level of anti-AngII antibodies and the age of patients (Spearman correlation; linear regression in red). (B) Proportion of male or female patients with high (Hi), low (Lo) or negative (−) levels of anti-AngII (χ² test). (C) Correlation between the level of anti-AngII antibodies and the BMI of patients (Spearman correlation; linear regression in red). (D) Correlation of the age of the patients with their level of anti-AngII (Spearman correlation; linear regression in black). U=underweight, N=normal weight, O=overweight, B=obese, SB=severely obese. (E) Proportion of patients of different weight categories that had no (−), low (Lo) of high (Hi) anti-AngII levels (χ² test).

FIGS. 11A-11F. Anti-AngII antibodies level does not strongly correlate with anti-RBD titers in COVID patients (N=115). (A) Anti-RBD titers (log₁₀) in hospitalized COVID patients as compared to control donors (sampled before the COVID pandemy; Mann-Whitney test). (B) Anti-RBD titers in COVID patients with negative (−), low (Lo) or high (Hi) levels of anti-AngII (Kruskall-Wallis test with Dunn's post-test). (C) Level of anti-AngII in COVID patients with negative (−), low (Lo) or high (Hi) titers of anti-RBD (Kruskall-Wallis test with Dunn's post-test). (D) Increase in anti-RBD titers in COVID patients between 1-10 and 11-20 days post-symptoms onset (DPSO) (N=25; Wilcoxon matched pairs signed rank test). (E) Increase in anti-AngII levels in COVID patients between 1-10 and 11-20 DPSO (N=25; Wilcoxon matched pairs signed rank test). (F) Levels of anti-AngII at late time (>45 DPSO) after symptoms onset (N=15 patients).

FIGS. 12A-12D. Epitopes targeted by the monoclonal anti-AngII clone E7 and B938M. Monoclonal anti-AngII were added onto a peptide array containing the full-length of Spike with 15-mers peptide with a 5 amino-acids overlap. (A) Sequences of the Spike linear epitopes targeted by the anti-AngII clone E7. (B) Sequences of the Spike linear epitopes targeted by the anti-AngII clone B938M. (C) Representative images of the positive spots on the Spike peptide arrays probed with the anti-AngII clone E7 or B938M, or the anti-IgG secondary control only. The spot J14 is the primary target of both anti-AngII monoclonal antibodies. (D) Sequences of the main targeted domains by the anti-AngII monoclonal antibodies that are highlighted in the 3D structure of Spike in FIG. 4F, with the corresponding amino-acid positions (aa #). All domains are targeted by both anti-AngII clones except the aa21-aa40 domain, which is a strong target of the clone B938M only, and aa786-804 which is targeted by clone E7 only. The peptide sequences in FIG. 12 correspond to the following SEQ ID NOS:

| FIG. | Peptide | SEQ ID NO |
|---|---|---|
| 12A | D7 | 2 |
| | D14 | 3 |
| | D20 | 4 |
| | D22 | 5 |
| | D23 | 6 |
| | E16 | 7 |
| | E17 | 8 |
| | F13 | 9 |
| | F21 | 10 |
| | F22 | 11 |
| | G14 | 12 |
| | G15 | 13 |
| | I4 | 14 |
| | I9 | 15 |
| | J13 | 16 |
| | J14 | 1 |
| 12B | A5 | 23 |
| | A6 | 24 |
| | D18 | 25 |
| | E15 | 26 |
| | E16 | 27 |
| | E17 | 28 |
| | E18 | 29 |
| | E19 | 30 |
| | F13 | 31 |
| | J11 | 32 |
| | J12 | 33 |
| | J14 | 34 |
| | J20 | 35 |
| 12D | aa21-aa40 | 36 |
| | aa447-aa468 | 37 |
| | aa551-aa585 | 38 |
| | aa786-aa804 | 39 |
| | aa1131-aa1160 | 40 |

FIG. 13. Binding sites of SARS-CoV-2 RBD and AngII on the human Angiotensin Converting Enzyme (ACE)-2. The binding of RBD on ACE-2 seems to occur at a different location than the catalytic site of AngII (PDB: 6M17 (10) by and AngII binding residues were determined from Guy et al. (11)).

FIG. 14A-14C. The Spike linear epitopes targeted by anti-AngII monoclonal antibodies are the same as the ones targeted by the plasma IgG of mice vaccinated with recombinant Spike or RBD proteins or of COVID convalescent patients. Highlighted colored regions are the main domains targeted by at least one anti-AngII monoclonal antibody. X-axes represent the position of linear epitopes covering the full-length of Spike. (A, B) Linear epitopes of Spike targeted by IgG antibodies in plasma of mice positive (+) or negative (−) for anti-AngII, upon immunization with recombinant Spike (A) or RBD (B) adjuvanted with MPLA/alum (plasma pooled from N=5 mice). (C) Linear epitopes of Spike targeted by IgG antibodies in plasma of COVID patients positive (+) or negative (−) for anti-AngII (plasma pooled from N=5 patients).

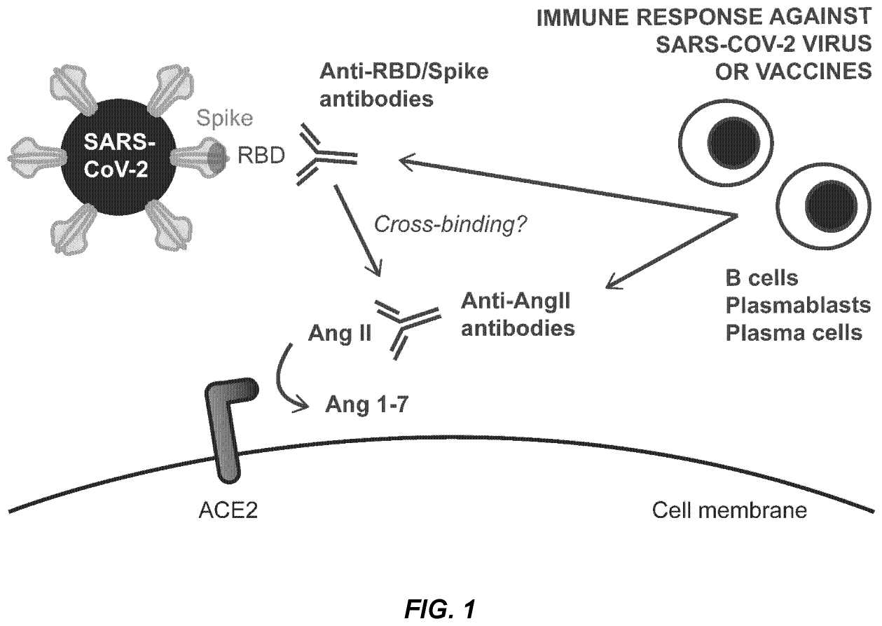

### DETAILED DESCRIPTION

The present disclosure is based, at least on part, on the surprising and unexpected discovery that natural infection with a coronavirus (e.g., SARS-CoV-2) and vaccination with SARS-CoV-2 Spike protein receptor binding domain (RBD) are each capable of inducing generation of antibodies that bind to angiotensin II (AngII). Aspects of the disclosure are directed to methods, systems, and compositions for detecting antibodies capable of binding to AngII in a biological sample from a subject. Such methods include, for example, detecting an antibody that binds to AngII in a biological sample from a subject having or having had a coronavirus, or from a subject having been administered a coronavirus vaccine. In some cases, the antibody further binds to SARS-CoV-2 Spike protein. Certain aspects are directed to compositions, including coronavirus (e.g., SARS-CoV-2) vaccine compositions, that do not induce generation of antibodies that bind to AngII, and methods for treating or preventing a coronavirus infection using such compositions. Such compositions include, for example, compositions comprising a portion of SARS-CoV-2 Spike protein, or a nucleic acid encoding for a portion of SARS-CoV-2 Spike protein, where the portion of SARS-CoV-2 Spike protein does not comprise SEQ ID NO:1. In some cases, the portion of SARS-CoV-2 Spike protein does not comprise one or more of SEQ ID NOs: 1-16 or 22-40.

Detection of the presence of anti-AngII antibodies may be used to determine an optimal treatment course for an infected patient or to determine the existence of any potential untoward response to vaccination in a subject. Such assays include enzyme-linked immunosorbent assays (ELISA), lateral flow assays, and the like. Thus, such diagnostics may be useful in the practice of medicine in infected subjects, especially in those with underlying conditions that may affect regulation of blood pressure such as hypertension, pulmonary hypertension, obesity, diabetes and renal disease. Such diagnostics may be useful in evaluation of vaccine candidates and immune response to vaccination.

Therapeutic antibodies may be used for treatment of a SARS-CoV-2 infection. Such antibodies may bind to Spike or RBD and serve to clear the virus and limit ongoing infection. These antibodies may be used as therapeutics to limit the severity of infection in patients who have been diagnosed as being infected. As disclosed herein, since antibodies generated by infection can bind to AngII, it may be important to screen therapeutic antibodies, whether discovered in a vaccinated animal or an infected and potentially convalesced patient or discovered via a laboratory method (including, for example, combinatorial methods such as phage display or yeast display), to exclude from pharmaceutical development those that bind to AngII. This applies to other inhibitors, such as aptamers, in which exclusion of binding to AngII is also important.

As disclosed herein, vaccines comprising SARS-CoV-2 Spike protein (“Spike”) and/or the RBD region may induce anti-AngII antibodies; this has important implications for vaccine design in that vaccines that do not generate an anti-AngII response may have greater benefit compared to those that do generate an anti-AngII response. Thus, identification of regions on Spike and/or RBD that generate an anti-AngII response may be useful, and vaccination with Spike or RBD antigens, either as proteins or as genetic sequences, that lack these domains may be superior immunogens. Thus, protein engineering approaches may be employed to mutate Spike or more specifically RBD in a vaccine to permit generation of an effective and neutralizing immune response while avoiding the generation of anti-AngII antibodies. In addition to selection of an optimal immunogen, i.e., the protein antigen or genetically-encoded antigen, determination of anti-AngII responses to vaccination may be important for selection of an optimal vaccine formulation, such as an optimal vaccine adjuvant. An adjuvant or formulation that generates strong neutralizing antibodies but avoids generation of anti-AngII antibodies may be clinically preferred compared to one that also generates anti-AngII antibody responses.

### I. Viruses

A. Coronaviruses

In particular embodiments, the virus is from the family Coronaviridae. Coronaviridae is a family of enveloped, positive-sense, single-stranded RNA viruses. Coronavirus is the common name for Coronaviridae and Orthocoronavirinae (also referred to as Coronavirinae). The family Coronaviridae is organized in 2 sub-families, 5 genera, 23 sub-genera and approximately 40 species. They are enveloped viruses having a positive-sense single-stranded RNA genome and a nucleocapsid having helical symmetry. The genome size of coronaviruses ranges from about 26-32 kilobases.

The present disclosure encompasses treatment or prevention of infection of any virus in the Coronaviridae family. In certain embodiments, the disclosure encompasses treatment or prevention of infection of any virus in the subfamily Coronavirinae and including the four genera, Alpha-, Beta-, Gamma-, and Deltacoronavirus. In specific embodiments, the disclosure encompasses treatment or prevention of infection of any virus in the genus of Betacoronavirus, including the subgenus Sarbecovirus and including the species of severe acute respiratory syndrome-related coronavirus. In specific embodiments, the disclosure encompasses treatment or prevention of infection of any virus in the species of severe acute respiratory syndrome-related coronavirus, including the strains severe acute respiratory syndrome coronavirus (SARS-CoV) and severe acute respiratory syndrome coronavirus 2 (SARS-CoV-2, the virus that causes COVID-19). The disclosure encompasses treatment or prevention of infection any isolate, strain, type (including Type A, Type B and Type C; Forster et al., 2020, PNAS, available on the World Wide Web at doi.org/10.1073/pnas.2004999117), cluster, or sub-cluster of the species of severe acute respiratory syndrome-related coronavirus, including at least SARS-CoV-2. In specific embodiments, the virus has a genome length between about 29000 to about 30000, between about 29100 and 29900, between about 29200 and 29900, between about 29300 and 29900, between about 29400 and 29900, between about 29500 and 29900, between about 29600 and 29900, between about 29700 and 29900, between about 29800 and 29900, or between about 29780 and 29900 base pairs in length.

Examples of specific SARS-CoV-2 viruses include the following listed in the NCBI GenBank® Database, and these GenBank® Accession sequences are incorporated by reference herein in their entirety: (a) LC534419 and LC534418 and LC528233 and LC529905 (examples of different strains from Japan); (b) MT281577 and MT226610 and NC_045512 and MN996531 and MN908947 (examples of different strains from China); (c) MT281530 (Iran); (d) MT126808 (Brazil); (e) MT020781 (Finland); (f) MT093571 (Sweden); (g) MT263074 (Peru); (h) MT292582 and MT292581 and MT292580 and MT292579 (examples of different strains from Spain); (i) examples from the United States, such as MT276331 (TX); MT276330 (FL); MT276328 (OR) MT276327 (GA); MT276325 (WA); MT276324 (CA); MT276323 (RI); MT188341 (MN); and (j) MT276598 (Israel). In particular embodiments, the disclosure encompasses treatment or prevention of infection of any of these or similar viruses, including viruses whose genome has at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. In particular embodiments, the disclosure encompasses treatment or prevention of infection of any of these or similar viruses, including viruses whose genome has its entire sequence that is greater than 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to any of these viruses. As one specific example, the present disclosure includes methods of treatment or prevention of infection of a virus having a genome sequence of SEQ ID NO:21 (represented by GenBank® Accession No. NC_045512; origin Wuhan, China) and any virus having a genome sequence with at least 80, 81, 82, 83, 84, 85, 86, 87, 88, 89, 90, 91, 92, 93, 94, 95, 96, 97, 98, 99, 99.1, 99.2, 99.3, 99.4, 99.5, 99.6, 99.7, 99.8, or 99.9% identity to SEQ ID NO:21.

SEQ ID NO:21 is represented by the following:

(SEQ ID NO: 21)
attaaaggtttataccttcccaggtaacaaaccaaccaactttcga
tctcttgtagatctgttctctaaacgaactttaaaatctgtgtgg
ctgtcactcggctgcatgcttagtgcactcacgcagtataattaa
taactaattactgtcgttgacaggacacgagtaactcgtctatct
tctgcaggctgcttacggtttcgtccgtgttgcagccgatcatca
gcacatctaggtttcgtccgggtgtgaccgaaaggtaagatggag
agccttgtccctggtttcaacgagaaaacacacgtccaactcagt
ttgcctgttttacaggttcgcgacgtgctcgtacgtggctttgga
gactccgtggaggaggtcttatcagaggcacgtcaacatcttaaa
gatggcacttgtggcttagtagaagttgaaaaaggcgttttgcct
caacttgaacagccctatgtgttcatcaaacgttcggatgctcga
actgcacctcatggtcatgttatggttgagctggtagcagaactc
gaaggcattcagtacggtcgtagtggtgagacacttggtgtcctt
gtccctcatgtgggcgaaataccagtggcttaccgcaaggttctt
cttcgtaagaacggtaataaaggagctggtggccatagttacggc
gccgatctaaagtcatttgacttaggcgacgagcttggcactgat
ccttatgaagattttcaagaaaactggaacactaaacatagcagt
ggtgttacccgtgaactcatgcgtgagcttaacggaggggcatac
actcgctatgtcgataacaacttctgtggccctgatggctaccct
cttgagtgcattaaagaccttctagcacgtgctggtaaagcttca
tgcactttgtccgaacaactggactttattgacactaagaggggt
gtatactgctgccgtgaacatgagcatgaaattgcttggtacacg
gaacgttctgaaaagagctatgaattgcagacaccttttgaaatt
aaattggcaaagaaatttgacaccttcaatggggaatgtccaaat
tttgtatttcccttaaattccataatcaagactattcaaccaagg
gttgaaaagaaaaagcttgatggctttatgggtagaattcgatct
gtctatccagttgcgtcaccaaatgaatgcaaccaaatgtgcctt
tcaactctcatgaagtgtgatcattgtggtgaaacttcatggcag
acgggcgattttgttaaagccacttgcgaattttgtggcactgag
aatttgactaaagaaggtgccactacttgtggttacttaccccaa
aatgctgttgttaaaatttattgtccagcatgtcacaattcagaa
gtaggacctgagcatagtcttgccgaataccataatgaatctggc
ttgaaaaccattcttcgtaagggggtcgcactattgcctttggag
gctgtgtgttctcttatgttggttgccataacaagtgtgcctatt
gggttccacgtgctagcgctaacataggttgtaaccatacaggtg
ttgttggagaaggttccgaaggtcttaatgacaaccttcttgaaa
tactccaaaaagagaaagtcaacatcaatattgttggtgacttta
aacttaatgaagagatcgccattattttggcatctttttctgctt
ccacaagtgcttttgtggaaactgtgaaaggtttggattataaag
cattcaaacaaattgttgaatcctgtggtaattttaaagttacaa
aaggaaaagctaaaaaaggtgcctggaatattggtgaacagaaat
caatactgagtcctctttatgcatttgcatcagaggctgctcgtg
ttgtacgatcaattttctcccgcactcttgaaactgctcaaaatt
ctgtgcgtgttttacagaaggccgctataacaatactagatggaa
tttcacagtattcactgagactcattgatgctatgatgttcacat
ctgatttggctactaacaatctagttgtaatggcctacattacag
gtggtgttgttcagttgacttcgcagtggctaactaacatctttg
gcactgtttatgaaaaactcaaacccgtccttgattggcttgaag
agaagtttaaggaaggtgtagagtttcttagagacggttgggaaa
ttgttaaatttatctcaacctgtgcttgtgaaattgtcggtggac
aaattgtcacctgtgcaaaggaaattaaggagagtgttcagacat
tctttaagcttgtaaataaatttttggctttgtgtgctgactcta
tcattattggtggagctaaacttaaagccttgaatttaggtgaaa
catttgtcacgcactcaaagggattgtacagaaagtgtgttaaat
ccagagaagaaactggcctactcatgcctctaaaagccccaaaag
aaattatcttcttagagggagaaacacttcccacagaagtgttaa
cagaggaagttgtcttgaaaactggtgatttacaaccattagaac
aacctactagtgaagctgttgaagctccattggttggtacaccag
tttgtattaacgggcttatgttgctcgaaatcaaagacacagaaa
agtactgtgcccttgcacctaatatgatggtaacaaacaatacct
tcacactcaaaggcggtgcaccaacaaaggttacttttggtgatg
acactgtgatagaagtgcaaggttacaagagtgtgaatatcactt
ttgaacttgatgaaaggattgataaagtacttaatgagaagtgct
ctgcctatacagttgaactcggtacagaagtaaatgagttcgcct
gtgttgtggcagatgctgtcataaaaactttgcaaccagtatctg
aattacttacaccactgggcattgatttagatgagtggagtatgg
ctacatactacttatttgatgagtctggtgagtttaaattggctt
cacatatgtattgttctttctaccctccagatgaggatgaagaag
aaggtgattgtgaagaagaagagtttgagccatcaactcaatatg
agtatggtactgaagatgattaccaaggtaaacctttggaatttg
gtgccacttctgctgctcttcaacctgaagaagagcaagaagaag
attggttagatgatgatagtcaacaaactgttggtcaacaagacg
gcagtgaggacaatcagacaactactattcaaacaattgttgagg
ttcaacctcaattagagatggaacttacaccagttgttcagacta
ttgaagtgaatagttttagtggttatttaaaacttactgacaatg
tatacattaaaaatgcagacattgtggaagaagctaaaaaggtaa
aaccaacagtggttgttaatgcagccaatgtttaccttaaacatg
gaggaggtgttgcaggagccttaaataaggctactaacaatgcca
tgcaagttgaatctgatgattacatagctactaatggaccactta
aagtgggtggtagttgtgttttaagcggacacaatcttgctaaac
actgtcttcatgttgtcggcccaaatgttaacaaaggtgaagaca
ttcaacttcttaagagtgcttatgaaaattttaatcagcacgaag
ttctacttgcaccattattatcagctggtatttttggtgctgacc
ctatacattctttaagagtttgtgtagatactgttcgcacaaatg
tctacttagctgtctttgataaaaatctctatgacaaacttgttt
caagctttttggaaatgaagagtgaaaagcaagttgaacaaaaga
tcgctgagattcctaaagaggaagttaagccatttataactgaaa
gtaaaccttcagttgaacagagaaaacaagatgataagaaaatca
aagcttgtgttgaagaagttacaacaactctggaagaaactaagt
tcctcacagaaaacttgttactttatattgacattaatggcaatc
ttcatccagattctgccactcttgttagtgacattgacatcactt
tcttaaagaaagatgctccatatatagtgggtgatgttgttcaag
agggtgttttaactgctgtggttatacctactaaaaaggctggtg
gcactactgaaatgctagcgaaagctttgagaaaagtgccaacag
acaattatataaccacttacccgggtcagggtttaaatggttaca
ctgtagaggaggcaaagacagtgcttaaaaagtgtaaaagtgcct
tttacattctaccatctattatctctaatgagaagcaagaaattc
ttggaactgtttcttggaatttgcgagaaatgcttgcacatgcag
aagaaacacgcaaattaatgcctgtctgtgtggaaactaaagcca
tagtttcaactatacagcgtaaatataagggtattaaaatacaag
agggtgtggttgattatggtgctagattttacttttacaccagta
aaacaactgtagcgtcacttatcaacacacttaacgatctaaatg
aaactcttgttacaatgccacttggctatgtaacacatggcttaa
atttggaagaagctgctcggtatatgagatctctcaaagtgccag
ctacagtttctgtttcttcacctgatgctgttacagcgtataatg
gttatcttacttcttcttctaaaacacctgaagaacattttattg
aaaccatctcacttgctggttcctataaagattggtcctattctg
gacaatctacacaactaggtatagaatttcttaagagaggtgata
aaagtgtatattacactagtaatcctaccacattccacctagatg
gtgaagttatcacctttgacaatcttaagacacttctttctttga
gagaagtgaggactattaaggtgtttacaacagtagacaacatta
acctccacacgcaagttgtggacatgtcaatgacatatggacaac
agtttggtccaacttatttggatggagctgatgttactaaaataa
aacctcataattcacatgaaggtaaaacattttatgttttaccta
atgatgacactctacgtgttgaggcttttgagtactaccacacaa
ctgatcctagttttctgggtaggtacatgtcagcattaaatcaca
ctaaaaagtggaaatacccacaagttaatggtttaacttctatta
aatgggcagataacaactgttatcttgccactgcattgttaacac
tccaacaaatagagttgaagtttaatccacctgctctacaagatg
cttattacagagcaagggctggtgaagctgctaacttttgtgcac
ttatcttagcctactgtaataagacagtaggtgagttaggtgatg
ttagagaaacaatgagttacttgtttcaacatgccaatttagatt
cttgcaaaagagtcttgaacgtggtgtgtaaaacttgtggacaac
agcagacaacccttaagggtgtagaagctgttatgtacatgggca
cactttcttatgaacaatttaagaaaggtgttcagataccttgta
cgtgtggtaaacaagctacaaaatatctagtacaacaggagtcac
cttttgttatgatgtcagcaccacctgctcagtatgaacttaagc
atggtacatttacttgtgctagtgagtacactggtaattaccagt
gtggtcactataaacatataacttctaaagaaactttgtattgca
tagacggtgctttacttacaaagtcctcagaatacaaaggtccta
ttacggatgttttctacaaagaaaacagttacacaacaaccataa
aaccagttacttataaattggatggtgttgtttgtacagaaattg
accctaagttggacaattattataagaaagacaattcttatttca
cagagcaaccaattgatcttgtaccaaaccaaccatatccaaacg
caagcttcgataattttaagtttgtatgtgataatatcaaatttg
ctgatgatttaaaccagttaactggttataagaaacctgcttcaa
gagagcttaaagttacatttttccctgacttaaatggtgatgtgg
tggctattgattataaacactacacaccctcttttaagaaaggag
ctaaattgttacataaacctattgtttggcatgttaacaatgcaa
ctaataaagccacgtataaaccaaatacctggtgtatacgttgtc
tttggagcacaaaaccagttgaaacatcaaattcgtttgatgtac
tgaagtcagaggacgcgcagggaatggataatcttgcctgcgaag
atctaaaaccagtctctgaagaagtagtggaaaatcctaccatac
agaaagacgttcttgagtgtaatgtgaaaactaccgaagttgtag
gagacattatacttaaaccagcaaataatagtttaaaaattacag
aagaggttggccacacagatctaatggctgcttatgtagacaatt
ctagtcttactattaagaaacctaatgaattatctagagtattag
gtttgaaaacccttgctactcatggtttagctgctgttaatagtg
tcccttgggatactatagctaattatgctaagccttttcttaaca
aagttgttagtacaactactaacatagttacacggtgtttaaacc
gtgtttgtactaattatatgccttatttctttactttattgctac
aattgtgtacttttactagaagtacaaattctagaattaaagcat
ctatgccgactactatagcaaagaatactgttaagagtgtcggta
aattttgtctagaggcttcatttaattatttgaagtcacctaatt
tttctaaactgataaatattataatttggtttttactattaagtg
tttgcctaggttctttaatctactcaaccgctgctttaggtgttt
taatgtctaatttaggcatgccttcttactgtactggttacagag
aaggctatttgaactctactaatgtcactattgcaacctactgta
ctggttctataccttgtagtgtttgtcttagtggtttagattctt
tagacacctatccttctttagaaactatacaaattaccatttcat
cttttaaatgggatttaactgcttttggcttagttgcagagtggt
ttttggcatatattcttttcactaggtttttctatgtacttggat
tggctgcaatcatgcaattgtttttcagctattttgcagtacatt
ttattagtaattcttggcttatgtggttaataattaatcttgtac
aaatggccccgatttcagctatggttagaatgtacatcttctttg
catcattttattatgtatggaaaagttatgtgcatgttgtagacg
gttgtaattcatcaacttgtatgatgtgttacaaacgtaatagag
caacaagagtcgaatgtacaactattgttaatggtgttagaaggt
ccttttatgtctatgctaatggaggtaaaggcttttgcaaactac
acaattggaattgtgttaattgtgatacattctgtgctggtagta
catttattagtgatgaagttgcgagagacttgtcactacagttta
aaagaccaataaatcctactgaccagtcttcttacatcgttgata
gtgttacagtgaagaatggttccatccatctttactttgataaag
ctggtcaaaagacttatgaaagacattctctctctcattttgtta
acttagacaacctgagagctaataacactaaaggttcattgccta
ttaatgttatagtttttgatggtaaatcaaaatgtgaagaatcat
ctgcaaaatcagcgtctgtttactacagtcagcttatgtgtcaac
ctatactgttactagatcaggcattagtgtctgatgttggtgata
gtgcggaagttgcagttaaaatgtttgatgcttacgttaatacgt
tttcatcaacttttaacgtaccaatggaaaaactcaaaacactag
ttgcaactgcagaagctgaacttgcaaagaatgtgtccttagaca
atgtcttatctacttttatttcagcagctcggcaagggtttgttg
attcagatgtagaaactaaagatgttgttgaatgtcttaaattgt
cacatcaatctgacatagaagttactggcgatagttgtaataact
atatgctcacctataacaaagttgaaaacatgacaccccgtgacc
ttggtgcttgtattgactgtagtgcgcgtcatattaatgcgcagg
tagcaaaaagtcacaacattgctttgatatggaacgttaaagatt
tcatgtcattgtctgaacaactacgaaaacaaatacgtagtgctg
ctaaaaagaataacttaccttttaagttgacatgtgcaactacta
gacaagttgttaatgttgtaacaacaaagatagcacttaagggtg
gtaaaattgttaataattggttgaagcagttaattaaagttacac
ttgtgttcctttttgttgctgctattttctatttaataacacctg
ttcatgtcatgtctaaacatactgacttttcaagtgaaatcatag
gatacaaggctattgatggtggtgtcactcgtgacatagcatcta
cagatacttgttttgctaacaaacatgctgattttgacacatggt
ttagccagcgtggtggtagttatactaatgacaaagcttgcccat
tgattgctgcagtcataacaagagaagtgggttttgtcgtgcctg
gtttgcctggcacgatattacgcacaactaatggtgactttttgc
atttcttacctagagtttttagtgcagttggtaacatctgttaca
caccatcaaaacttatagagtacactgactttgcaacatcagctt
gtgttttggctgctgaatgtacaatttttaaagatgcttctggta
agccagtaccatattgttatgataccaatgtactagaaggttctg
ttgcttatgaaagtttacgccctgacacacgttatgtgctcatgg
atggctctattattcaatttcctaacacctaccttgaaggttctg
ttagagtggtaacaacttttgattctgagtactgtaggcacggca
cttgtgaaagatcagaagctggtgtttgtgtatctactagtggta
gatgggtacttaacaatgattattacagatctttaccaggagttt
tctgtggtgtagatgctgtaaatttacttactaatatgtttacac
cactaattcaacctattggtgctttggacatatcagcatctatag
tagctggtggtattgtagctatcgtagtaacatgccttgcctact
attttatgaggtttagaagagcttttggtgaatacagtcatgtag
ttgcctttaatactttactattccttatgtcattcactgtactct
gtttaacaccagtttactcattcttacctggtgtttattctgtta
tttacttgtacttgacattttatcttactaatgatgtttcttttt
tagcacatattcagtggatggttatgttcacacctttagtacctt
tctggataacaattgcttatatcatttgtatttccacaaagcatt
tctattggttctttagtaattacctaaagagacgtgtagtcttta
atggtgtttcctttagtacttttgaagaagctgcgctgtgcacct
ttttgttaaataaagaaatgtatctaaagttgcgtagtgatgtgc
tattacctcttacgcaatataatagatacttagctctttataata
agtacaagtattttagtggagcaatggatacaactagctacagag
aagctgcttgttgtcatctcgcaaaggctctcaatgacttcagta
actcaggttctgatgttctttaccaaccaccacaaacctctatca
cctcagctgttttgcagagtggttttagaaaaatggcattcccat
ctggtaaagttgagggttgtatggtacaagtaacttgtggtacaa
ctacacttaacggtctttggcttgatgacgtagtttactgtccaa
gacatgtgatctgcacctctgaagacatgcttaaccctaattatg
aagatttactcattcgtaagtctaatcataatttcttggtacagg
ctggtaatgttcaactcagggttattggacattctatgcaaaatt
gtgtacttaagcttaaggttgatacagccaatcctaagacaccta
agtataagtttgttcgcattcaaccaggacagactttttcagtgt
tagcttgttacaatggttcaccatctggtgtttaccaatgtgcta
tgaggcccaatttcactattaagggttcattccttaatggttcat
gtggtagtgttggttttaacatagattatgactgtgtctcttttt
gttacatgcaccatatggaattaccaactggagttcatgctggca
cagacttagaaggtaacttttatggaccttttgttgacaggcaaa
cagcacaagcagctggtacggacacaactattacagttaatgttt
tagcttggttgtacgctgctgttataaatggagacaggtggtttc
tcaatcgatttaccacaactcttaatgactttaaccttgtggcta
tgaagtacaattatgaacctctaacacaagaccatgttgacatac
taggacctctttctgctcaaactggaattgccgttttagatatgt
gtgcttcattaaaagaattactgcaaaatggtatgaatggacgta
ccatattgggtagtgctttattagaagatgaatttacaccttttg
atgttgttagacaatgctcaggtgttactttccaaagtgcagtga
aaagaacaatcaagggtacacaccactggttgttactcacaattt
tgacttcacttttagttttagtccagagtactcaatggtctttgt
tcttttttttgtatgaaaatgcctttttaccttttgctatgggta
ttattgctatgtctgcttttgcaatgatgtttgtcaaacataagc
atgcatttctctgtttgtttttgttaccttctcttgccactgtag
cttattttaatatggtctatatgcctgctagttgggtgatgcgta
ttatgacatggttggatatggttgatactagtttgtctggtttta
agctaaaagactgtgttatgtatgcatcagctgtagtgttactaa
tccttatgacagcaagaactgtgtatgatgatggtgctaggagag
tgtggacacttatgaatgtcttgacactcgtttataaagtttatt
atggtaatgctttagatcaagccatttccatgtgggctcttataa
tctctgttacttctaactactcaggtgtagttacaactgtcatgt
ttttggccagaggtattgtttttatgtgtgttgagtattgcccta
ttttcttcataactggtaatacacttcagtgtataatgctagttt
attgtttcttaggctatttttgtacttgttactttggcctctttt
gtttactcaaccgctactttagactgactcttggtgtttatgatt
acttagtttctacacaggagtttagatatatgaattcacagggac
tactcccacccaagaatagcatagatgccttcaaactcaacatta
aattgttgggtgttggtggcaaaccttgtatcaaagtagccactg
tacagtctaaaatgtcagatgtaaagtgcacatcagtagtcttac
tctcagttttgcaacaactcagagtagaatcatcatctaaattgt
gggctcaatgtgtccagttacacaatgacattctcttagctaaag
atactactgaagcctttgaaaaaatggtttcactactttctgttt
tgctttccatgcagggtgctgtagacataaacaagctttgtgaag
aaatgctggacaacagggcaaccttacaagctatagcctcagagt
ttagttcccttccatcatatgcagcttttgctactgctcaagaag
cttatgagcaggctgttgctaatggtgattctgaagttgttctta
aaaagttgaagaagtctttgaatgtggctaaatctgaatttgacc
gtgatgcagccatgcaacgtaagttggaaaagatggctgatcaag
ctatgacccaaatgtataaacaggctagatctgaggacaagaggg
caaaagttactagtgctatgcagacaatgcttttcactatgctta
gaaagttggataatgatgcactcaacaacattatcaacaatgcaa
gagatggttgtgttcccttgaacataatacctcttacaacagcag
ccaaactaatggttgtcataccagactataacacatataaaaata
cgtgtgatggtacaacatttacttatgcatcagcattgtgggaaa
tccaacaggttgtagatgcagatagtaaaattgttcaacttagtg
aaattagtatggacaattcacctaatttagcatggcctcttattg
taacagctttaagggccaattctgctgtcaaattacagaataatg
agcttagtcctgttgcactacgacagatgtcttgtgctgccggta
ctacacaaactgcttgcactgatgacaatgcgttagcttactaca
acacaacaaagggaggtaggtttgtacttgcactgttatccgatt
tacaggatttgaaatgggctagattccctaagagtgatggaactg
gtactatctatacagaactggaaccaccttgtaggtttgttacag
acacacctaaaggtcctaaagtgaagtatttatactttattaaag
gattaaacaacctaaatagaggtatggtacttggtagtttagctg
ccacagtacgtctacaagctggtaatgcaacagaagtgcctgcca
attcaactgtattatctttctgtgcttttgctgtagatgctgcta
aagcttacaaagattatctagctagtgggggacaaccaatcacta
attgtgttaagatgttgtgtacacacactggtactggtcaggcaa
taacagttacaccggaagccaatatggatcaagaatcctttggtg
gtgcatcgtgttgtctgtactgccgttgccacatagatcatccaa
atcctaaaggattttgtgacttaaaaggtaagtatgtacaaatac
ctacaacttgtgctaatgaccctgtgggttttacacttaaaaaca
cagtctgtaccgtctgcggtatgtggaaaggttatggctgtagtt
gtgatcaactccgcgaacccatgcttcagtcagctgatgcacaat
cgtttttaaacgggtttgcggtgtaagtgcagcccgtcttacacc
gtgcggcacaggcactagtactgatgtcgtatacagggcttttga
catctacaatgataaagtagctggttttgctaaattcctaaaaac
taattgttgtcgcttccaagaaaaggacgaagatgacaatttaat
tgattcttactttgtagttaagagacacactttctctaactacca
acatgaagaaacaatttataatttacttaaggattgtccagctgt
tgctaaacatgacttctttaagtttagaatagacggtgacatggt
accacatatatcacgtcaacgtcttactaaatacacaatggcaga
cctcgtctatgctttaaggcattttgatgaaggtaattgtgacac
attaaaagaaatacttgtcacatacaattgttgtgatgatgatta
tttcaataaaaaggactggtatgattttgtagaaaacccagatat
attacgcgtatacgccaacttaggtgaacgtgtacgccaagcttt
gttaaaaacagtacaattctgtgatgccatgcgaaatgctggtat
tgttggtgtactgacattagataatcaagatctcaatggtaactg
gtatgatttcggtgatttcatacaaaccacgccaggtagtggagt
tcctgttgtagattcttattattcattgttaatgcctatattaac
cttgaccagggctttaactgcagagtcacatgttgacactgactt
aacaaagccttacattaagtgggatttgttaaaatatgacttcac
ggaagagaggttaaaactctttgaccgttattttaaatattggga
tcagacataccacccaaattgtgttaactgtttggatgacagatg
cattctgcattgtgcaaactttaatgttttattctctacagtgtt
cccacctacaagttttggaccactagtgagaaaaatatttgttga
tggtgttccatttgtagtttcaactggataccacttcagagagct
aggtgttgtacataatcaggatgtaaacttacatagctctagact
tagttttaaggaattacttgtgtatgctgctgaccctgctatgca
cgctgcttctggtaatctattactagataaacgcactacgtgctt
ttcagtagctgcacttactaacaatgttgcttttcaaactgtcaa
acccggtaattttaacaaagacttctatgactttgctgtgtctaa
gggtttctttaaggaaggaagttctgttgaattaaaacacttctt
ctttgctcaggatggtaatgctgctatcagcgattatgactacta
tcgttataatctaccaacaatgtgtgatatcagacaactactatt
tgtagttgaagttgttgataagtactttgattgttacgatggtgg
ctgtattaatgctaaccaagtcatcgtcaacaacctagacaaatc
agctggttttccatttaataaatggggtaaggctagactttatta
tgattcaatgagttatgaggatcaagatgcacttttcgcatatac
aaaacgtaatgtcatccctactataactcaaatgaatcttaagta
tgccattagtgcaaagaatagagctcgcaccgtagctggtgtctc
tatctgtagtactatgaccaatagacagtttcatcaaaaattatt
gaaatcaatagccgccactagaggagctactgtagtaattggaac
aagcaaattctatggtggttggcacaacatgttaaaaactgttta
tagtgatgtagaaaaccctcaccttatgggttgggattatcctaa
atgtgatagagccatgcctaacatgcttagaattatggcctcact
tgttcttgctcgcaaacatacaacgtgttgtagcttgtcacaccg
tttctatagattagctaatgagtgtgctcaagtattgagtgaaat
ggtcatgtgtggcggttcactatatgttaaaccaggtggaacctc
atcaggagatgccacaactgcttatgctaatagtgtttttaacat
ttgtcaagctgtcacggccaatgttaatgcacttttatctactga
tggtaacaaaattgccgataagtatgtccgcaatttacaacacag
actttatgagtgtctctatagaaatagagatgttgacacagactt
tgtgaatgagttttacgcatatttgcgtaaacatttctcaatgat
gatactctctgacgatgctgttgtgtgtttcaatagcacttatgc
atctcaaggtctagtggctagcataaagaactttaagtcagttct
ttattatcaaaacaatgtttttatgtctgaagcaaaatgttggac
tgagactgaccttactaaaggacctcatgaattttgctctcaaca
tacaatgctagttaaacagggtgatgattatgtgtaccttcctta
cccagatccatcaagaatcctaggggccggctgttttgtagatga
tatcgtaaaaacagatggtacacttatgattgaacggttcgtgtc
tttagctatagatgcttacccacttactaaacatcctaatcagga
gtatgctgatgtctttcatttgtacttacaatacataagaaagct
acatgatgagttaacaggacacatgttagacatgtattctgttat
gcttactaatgataacacttcaaggtattgggaacctgagtttta
tgaggctatgtacacaccgcatacagtcttacaggctgttggggc
ttgtgttctttgcaattcacagacttcattaagatgtggtgcttg
catacgtagaccattcttatgttgtaaatgctgttacgaccatgt
catatcaacatcacataaattagtcttgtctgttaatccgtatgt
ttgcaatgctccaggttgtgatgtcacagatgtgactcaacttta
cttaggaggtatgagctattattgtaaatcacataaaccacccat
tagttttccattgtgtgctaatggacaagtttttggtttatataa
aaatacatgtgttggtagcgataatgttactgactttaatgcaat
tgcaacatgtgactggacaaatgctggtgattacattttagctaa
cacctgtactgaaagactcaagctttttgcagcagaaacgctcaa
agctactgaggagacatttaaactgtcttatggtattgctactgt
acgtgaagtgctgtctgacagagaattacatctttcatgggaagt
tggtaaacctagaccaccacttaaccgaaattatgtctttactgg
ttatcgtgtaactaaaaacagtaaagtacaaataggagagtacac
ctttgaaaaaggtgactatggtgatgctgttgtttaccgaggtac
aacaacttacaaattaaatgttggtgattattttgtgctgacatc
acatacagtaatgccattaagtgcacctacactagtgccacaaga
gcactatgttagaattactggcttatacccaacactcaatatctc
agatgagttttctagcaatgttgcaaattatcaaaaggttggtat
gcaaaagtattctacactccagggaccacctggtactggtaagag
tcattttgctattggcctagctctctactacccttctgctcgcat
agtgtatacagcttgctctcatgccgctgttgatgcactatgtga
gaaggcattaaaatatttgcctatagataaatgtagtagaattat
acctgcacgtgctcgtgtagagtgttttgataaattcaaagtgaa
ttcaacattagaacagtatgtcttttgtactgtaaatgcattgcc
tgagacgacagcagatatagttgtctttgatgaaatttcaatggc
cacaaattatgatttgagtgttgtcaatgccagattacgtgctaa
gcactatgtgtacattggcgaccctgctcaattacctgcaccacg
cacattgctaactaagggcacactagaaccagaatatttcaattc
agtgtgtagacttatgaaaactataggtccagacatgttcctcgg
aacttgtcggcgttgtcctgctgaaattgttgacactgtgagtgc
tttggtttatgataataagcttaaagcacataaagacaaatcagc
tcaatgctttaaaatgttttataagggtgttatcacgcatgatgt
ttcatctgcaattaacaggccacaaataggcgtggtaagagaatt
ccttacacgtaaccctgcttggagaaaagctgtctttatttcacc
ttataattcacagaatgctgtagcctcaaagattttgggactacc
aactcaaactgttgattcatcacagggctcagaatatgactatgt
catattcactcaaaccactgaaacagctcactcttgtaatgtaaa
cagatttaatgttgctattaccagagcaaaagtaggcatactttg
cataatgtctgatagagacctttatgacaagttgcaatttacaag
tcttgaaattccacgtaggaatgtggcaactttacaagctgaaaa
tgtaacaggactctttaaagattgtagtaaggtaatcactgggtt
acatcctacacaggcacctacacacctcagtgttgacactaaatt
caaaactgaaggtttatgtgttgacatacctggcatacctaagga
catgacctatagaagactcatctctatgatgggttttaaaatgaa
ttatcaagttaatggttaccctaacatgtttatcacccgcgaaga
agctataagacatgtacgtgcatggattggcttcgatgtcgaggg
gtgtcatgctactagagaagctgttggtaccaatttacctttaca
gctaggtttttctacaggtgttaacctagttgctgtacctacagg
ttatgttgatacacctaataatacagatttttccagagttagtgc
taaaccaccgcctggagatcaatttaaacacctcataccacttat
gtacaaaggacttccttggaatgtagtgcgtataaagattgtaca
aatgttaagtgacacacttaaaaatctctctgacagagtcgtatt
tgtcttatgggcacatggctttgagttgacatctatgaagtattt
tgtgaaaataggacctgagcgcacctgttgtctatgtgatagacg
tgccacatgcttttccactgcttcagacacttatgcctgttggca
tcattctattggatttgattacgtctataatccgtttatgattga
tgttcaacaatggggttttacaggtaacctacaaagcaaccatga
tctgtattgtcaagtccatggtaatgcacatgtagctagttgtga
tgcaatcatgactaggtgtctagctgtccacgagtgctttgttaa
gcgtgttgactggactattgaatatcctataattggtgatgaact
gaagattaatgcggcttgtagaaaggttcaacacatggttgttaa
agctgcattattagcagacaaattcccagttcttcacgacattgg
taaccctaaagctattaagtgtgtacctcaagctgatgtagaatg
gaagttctatgatgcacagccttgtagtgacaaagcttataaaat
agaagaattattctattcttatgccacacattctgacaaattcac
agatggtgtatgcctattttggaattgcaatgtcgatagatatcc
tgctaattccattgtttgtagatttgacactagagtgctatctaa
ccttaacttgcctggttgtgatggtggcagtttgtatgtaaataa
acatgcattccacacaccagcttttgataaaagtgcttttgttaa
tttaaaacaattaccatttttctattactctgacagtccatgtga
gtctcatggaaaacaagtagtgtcagatatagattatgtaccact
aaagtctgctacgtgtataacacgttgcaatttaggtggtgctgt
ctgtagacatcatgctaatgagtacagattgtatctcgatgctta
taacatgatgatctcagctggctttagcttgtgggtttacaaaca
atttgatacttataacctctggaacacttttacaagacttcagag
tttagaaaatgtggcttttaatgttgtaaataagggacactttga
tggacaacagggtgaagtaccagtttctatcattaataacactgt
ttacacaaaagttgatggtgttgatgtagaattgtttgaaaataa
aacaacattacctgttaatgtagcatttgagctttgggctaagcg
caacattaaaccagtaccagaggtgaaaatactcaataatttggg
tgtggacattgctgctaatactgtgatctgggactacaaaagaga
tgctccagcacatatatctactattggtgtttgttctatgactga
catagccaagaaaccaactgaaacgatttgtgcaccactcactgt
cttttttgatggtagagttgatggtcaagtagacttatttagaaa
tgcccgtaatggtgttcttattacagaaggtagtgttaaaggttt
acaaccatctgtaggtcccaaacaagctagtcttaatggagtcac
attaattggagaagccgtaaaaacacagttcaattattataagaa
agttgatggtgttgtccaacaattacctgaaacttactttactca
gagtagaaatttacaagaatttaaacccaggagtcaaatggaaat
tgatttcttagaattagctatggatgaattcattgaacggtataa
attagaaggctatgccttcgaacatatcgtttatggagattttag
tcatagtcagttaggtggtttacatctactgattggactagctaa
acgttttaaggaatcaccttttgaattagaagattttattcctat
ggacagtacagttaaaaactatttcataacagatgcgcaaacagg
ttcatctaagtgtgtgtgttctgttattgatttattacttgatga
ttttgttgaaataataaaatcccaagatttatctgtagtttctaa
ggttgtcaaagtgactattgactatacagaaatttcatttatgct
ttggtgtaaagatggccatgtagaaacattttacccaaaattaca
atctagtcaagcgtggcaaccgggtgttgctatgcctaatcttta
caaaatgcaaagaatgctattagaaaagtgtgaccttcaaaatta
tggtgatagtgcaacattacctaaaggcataatgatgaatgtcgc
aaaatatactcaactgtgtcaatatttaaacacattaacattagc
tgtaccctataatatgagagttatacattttggtgctggttctga
taaaggagttgcaccaggtacagctgttttaagacagtggttgcc
tacgggtacgctgcttgtcgattcagatcttaatgactttgtctc
tgatgcagattcaactttgattggtgattgtgcaactgtacatac
agctaataaatgggatctcattattagtgatatgtacgaccctaa
gactaaaaatgttacaaaagaaaatgactctaaagagggtttttt
cacttacatttgtgggtttatacaacaaaagctagctcttggagg
ttccgtggctataaagataacagaacattcttggaatgctgatct
ttataagctcatgggacacttcgcatggtggacagcctttgttac
taatgtgaatgcgtcatcatctgaagcatttttaattggatgtaa
ttatcttggcaaaccacgcgaacaaatagatggttatgtcatgca
tgcaaattacatattttggaggaatacaaatccaattcagttgtc
ttcctattctttatttgacatgagtaaatttccccttaaattaag
gggtactgctgttatgtctttaaaagaaggtcaaatcaatgatat
gattttatctcttcttagtaaaggtagacttataattagagaaaa
caacagagttgttatttctagtgatgttcttgttaacaactaaac
gaacaatgtttgtttttcttgttttattgccactagtctctagtc
agtgtgttaatcttacaaccagaactcaattaccccctgcataca
ctaattctttcacacgtggtgtttattaccctgacaaagttttca
gatcctcagttttacattcaactcaggacttgttcttacctttct
tttccaatgttacttggttccatgctatacatgtctctgggacca
atggtactaagaggtttgataaccctgtcctaccatttaatgatg
gtgtttattttgcttccactgagaagtctaacataataagaggct
ggatttttggtactactttagattcgaagacccagtccctactta
ttgttaataacgctactaatgttgttattaaagtctgtgaatttc
aattttgtaatgatccatttttgggtgtttattaccacaaaaaca
acaaaagttggatggaaagtgagttcagagtttattctagtgcga
ataattgcacttttgaatatgtctctcagccttttcttatggacc
ttgaaggaaaacagggtaatttcaaaaatcttagggaatttgtgt
ttaagaatattgatggttattttaaaatatattctaagcacacgc
ctattaatttagtgcgtgatctccctcagggtttttcggctttag
aaccattggtagatttgccaataggtattaacatcactaggtttc
aaactttacttgctttacatagaagttatttgactcctggtgatt
cttcttcaggttggacagctggtgctgcagcttattatgtgggtt
atcttcaacctaggacttttctattaaaatataatgaaaatggaa
ccattacagatgctgtagactgtgcacttgaccctctctcagaaa
caaagtgtacgttgaaatccttcactgtagaaaaaggaatctatc
aaacttctaactttagagtccaaccaacagaatctattgttagat
ttcctaatattacaaacttgtgcccttttggtgaagtttttaacg
ccaccagatttgcatctgtttatgcttggaacaggaagagaatca
gcaactgtgttgctgattattctgtcctatataattccgcatcat
tttccacttttaagtgttatggagtgtctcctactaaattaaatg
atctctgctttactaatgtctatgcagattcatttgtaattagag
gtgatgaagtcagacaaatcgctccagggcaaactggaaagattg
ctgattataattataaattaccagatgattttacaggctgcgtta
tagcttggaattctaacaatcttgattctaaggttggtggtaatt
ataattacctgtatagattgtttaggaagtctaatctcaaacctt
ttgagagagatatttcaactgaaatctatcaggccggtagcacac
cttgtaatggtgttgaaggttttaattgttactttcctttacaat
catatggtttccaacccactaatggtgttggttaccaaccataca
gagtagtagtactttcttttgaacttctacatgcaccagcaactg
tttgtggacctaaaaagtctactaatttggttaaaaacaaatgtg
tcaatttcaacttcaatggtttaacaggcacaggtgttcttactg
agtctaacaaaaagtttctgcctttccaacaatttggcagagaca
ttgctgacactactgatgctgtccgtgatccacagacacttgaga
ttcttgacattacaccatgttcttttggtggtgtcagtgttataa
caccaggaacaaatacttctaaccaggttgctgttctttatcagg
atgttaactgcacagaagtccctgttgctattcatgcagatcaac
ttactcctacttggcgtgtttattctacaggttctaatgtttttc
aaacacgtgcaggctgtttaataggggctgaacatgtcaacaact
catatgagtgtgacatacccattggtgcaggtatatgcgctagtt
atcagactcagactaattctcctcggcgggcacgtagtgtagcta
gtcaatccatcattgcctacactatgtcacttggtgcagaaaatt
cagttgcttactctaataactctattgccatacccacaaatttta
ctattagtgttaccacagaaattctaccagtgtctatgaccaaga
catcagtagattgtacaatgtacatttgtggtgattcaactgaat
gcagcaatcttttgttgcaatatggcagtttttgtacacaattaa
accgtgctttaactggaatagctgttgaacaagacaaaaacaccc
aagaagtttttgcacaagtcaaacaaatttacaaaacaccaccaa
ttaaagattttggtggttttaatttttcacaaatattaccagatc
catcaaaaccaagcaagaggtcatttattgaagatctacttttca
acaaagtgacacttgcagatgctggcttcatcaaacaatatggtg
attgccttggtgatattgctgctagagacctcatttgtgcacaaa
agtttaacggccttactgttttgccacctttgctcacagatgaaa
tgattgctcaatacacttctgcactgttagcgggtacaatcactt
ctggttggacctttggtgcaggtgctgcattacaaataccatttg
ctatgcaaatggcttataggtttaatggtattggagttacacaga
atgttctctatgagaaccaaaaattgattgccaaccaatttaata
gtgctattggcaaaattcaagactcactttcttccacagcaagtg
cacttggaaaacttcaagatgtggtcaaccaaaatgcacaagctt
taaacacgcttgttaaacaacttagctccaattttggtgcaattt
caagtgttttaaatgatatcctttcacgtcttgacaaagttgagg
ctgaagtgcaaattgataggttgatcacaggcagacttcaaagtt
tgcagacatatgtgactcaacaattaattagagctgcagaaatca
gagcttctgctaatcttgctgctactaaaatgtcagagtgtgtac
ttggacaatcaaaaagagttgatttttgtggaaagggctatcatc
ttatgtccttccctcagtcagcacctcatggtgtagtcttcttgc
atgtgacttatgtccctgcacaagaaaagaacttcacaactgctc
ctgccatttgtcatgatggaaaagcacactttcctcgtgaaggtg
tctttgtttcaaatggcacacactggtttgtaacacaaaggaatt
tttatgaaccacaaatcattactacagacaacacatttgtgtctg
gtaactgtgatgttgtaataggaattgtcaacaacacagtttatg
atcctttgcaacctgaattagactcattcaaggaggagttagata
aatattttaagaatcatacatcaccagatgttgatttaggtgaca
tctctggcattaatgcttcagttgtaaacattcaaaaagaaattg
accgcctcaatgaggttgccaagaatttaaatgaatctctcatcg
atctccaagaacttggaaagtatgagcagtatataaaatggccat
ggtacatttggctaggttttatagctggcttgattgccatagtaa
tggtgacaattatgctttgctgtatgaccagttgctgtagttgtc
tcaagggctgttgttcttgtggatcctgctgcaaatttgatgaag
acgactctgagccagtgctcaaaggagtcaaattacattacacat
aaacgaacttatggatttgtttatgagaatcttcacaattggaac
tgtaactttgaagcaaggtgaaatcaaggatgctactccttcaga
ttttgttcgcgctactgcaacgataccgatacaagcctcactccc
tttcggatggcttattgttggcgttgcacttcttgctgtttttca
gagcgcttccaaaatcataaccctcaaaaagagatggcaactagc
actctccaagggtgttcactttgtttgcaacttgctgttgttgtt
tgtaacagtttactcacaccttttgctcgttgctgctggccttga
agccccttttctctatctttatgctttagtctacttcttgcagag
tataaactttgtaagaataataatgaggctttggctttgctggaa
atgccgttccaaaaacccattactttatgatgccaactattttct
ttgctggcatactaattgttacgactattgtataccttacaatag
tgtaacttcttcaattgtcattacttcaggtgatggcacaacaag
tcctatttctgaacatgactaccagattggtggttatactgaaaa
atgggaatctggagtaaaagactgtgttgtattacacagttactt
cacttcagactattaccagctgtactcaactcaattgagtacaga
cactggtgttgaacatgttaccttcttcatctacaataaaattgt
tgatgagcctgaagaacatgtccaaattcacacaatcgacggttc
atccggagttgttaatccagtaatggaaccaatttatgatgaacc
gacgacgactactagcgtgcctttgtaagcacaagctgatgagta
cgaacttatgtactcattcgtttcggaagagacaggtacgttaat
agttaatagcgtacttctttttcttgctttcgtggtattcttgct
agttacactagccatccttactgcgcttcgattgtgtgcgtactg
ctgcaatattgttaacgtgagtcttgtaaaaccttctttttacgt
ttactctcgtgttaaaaatctgaattcttctagagttcctgatct
tctggtctaaacgaactaaatattatattagtttttctgtttgga
actttaattttagccatggcagattccaacggtactattaccgtt
gaagagcttaaaaagctccttgaacaatggaacctagtaataggt
ttcctattccttacatggatttgtcttctacaatttgcctatgcc
aacaggaataggtttttgtatataattaagttaattttcctctgg
ctgttatggccagtaactttagcttgttttgtgcttgctgctgtt
tacagaataaattggatcaccggtggaattgctatcgcaatggct
tgtcttgtaggcttgatgtggctcagctacttcattgcttctttc
agactgtttgcgcgtacgcgttccatgtggtcattcaatccagaa
actaacattcttctcaacgtgccactccatggcactattctgacc
agaccgcttctagaaagtgaactcgtaatcggagctgtgatcctt
cgtggacatcttcgtattgctggacaccatctaggacgctgtgac
atcaaggacctgcctaaagaaatcactgttgctacatcacgaacg
ctttcttattacaaattgggagcttcgcagcgtgtagcaggtgac
tcaggttttgctgcatacagtcgctacaggattggcaactataaa
ttaaacacagaccattccagtagcagtgacaatattgctttgctt
gtacagtaagtgacaacagatgtttcatctcgttgactttcaggt
tactatagcagagatattactaattattatgaggacttttaaagt
ttccatttggaatcttgattacatcataaacctcataattaaaaa
tttatctaagtcactaactgagaataaatattctcaattagatga
agagcaaccaatggagattgattaaacgaacatgaaaattattct
tttcttggcactgataacactcgctacttgtgagctttatcacta
ccaagagtgtgttagaggtacaacagtacttttaaaagaaccttg
ctcttctggaacatacgagggcaattcaccatttcatcctctagc
tgataacaaatttgcactgacttgctttagcactcaatttgcttt
tgcttgtcctgacggcgtaaaacacgtctatcagttacgtgccag
atcagtttcacctaaactgttcatcagacaagaggaagttcaaga
actttactctccaatttttcttattgttgcggcaatagtgtttat
aacactttgcttcacactcaaaagaaagacagaatgattgaactt
tcattaattgacttctatttgtgctttttagcctttctgctattc
cttgttttaattatgcttattatcttttggttctcacttgaactg
caagatcataatgaaacttgtcacgcctaaacgaacatgaaattt
cttgttttcttaggaatcatcacaactgtagctgcatttcaccaa
gaatgtagtttacagtcatgtactcaacatcaaccatatgtagtt
gatgacccgtgtcctattcacttctattctaaatggtatattaga
gtaggagctagaaaatcagcacctttaattgaattgtgcgtggat
gaggctggttctaaatcacccattcagtacatcgatatcggtaat
tatacagtttcctgtttaccttttacaattaattgccaggaacct
aaattgggtagtcttgtagtgcgttgttcgttctatgaagacttt
ttagagtatcatgacgttcgtgttgttttagatttcatctaaacg
aacaaactaaaatgtctgataatggaccccaaaatcagcgaaatg
caccccgcattacgtttggtggaccctcagattcaactggcagta
accagaatggagaacgcagtggggcgcgatcaaaacaacgtcggc
cccaaggtttacccaataatactgcgtcttggttcaccgctctca
ctcaacatggcaaggaagaccttaaattccctcgaggacaaggcg
ttccaattaacaccaatagcagtccagatgaccaaattggctact
accgaagagctaccagacgaattcgtggtggtgacggtaaaatga
aagatctcagtccaagatggtatttctactacctaggaactgggc
cagaagctggacttccctatggtgctaacaaagacggcatcatat
gggttgcaactgagggagccttgaatacaccaaaagatcacattg
gcacccgcaatcctgctaacaatgctgcaatcgtgctacaacttc
ctcaaggaacaacattgccaaaaggcttctacgcagaagggagca
gaggcggcagtcaagcctcttctcgttcctcatcacgtagtcgca
acagttcaagaaattcaactccaggcagcagtaggggaacttctc
ctgctagaatggctggcaatggcggtgatgctgctcttgctttgc
tgctgcttgacagattgaaccagcttgagagcaaaatgtctggta
aaggccaacaacaacaaggccaaactgtcactaagaaatctgctg
ctgaggcttctaagaagcctcggcaaaaacgtactgccactaaag
catacaatgtaacacaagctttcggcagacgtggtccagaacaaa
cccaaggaaattttggggaccaggaactaatcagacaaggaactg
attacaaacattggccgcaaattgcacaatttgcccccagcgctt
cagcgttcttcggaatgtcgcgcattggcatggaagtcacacctt
cgggaacgtggttgacctacacaggtgccatcaaattggatgaca
aagatccaaatttcaaagatcaagtcattttgctgaataagcata
ttgacgcatacaaaacattcccaccaacagagcctaaaaaggaca
aaaagaagaaggctgatgaaactcaagccttaccgcagagacaga
agaaacagcaaactgtgactcttcttcctgctgcagatttggatg
atttctccaaacaattgcaacaatccatgagcagtgctgactcaa
ctcaggcctaaactcatgcagaccacacaaggcagatgggctata
taaacgttttcgcttttccgtttacgatatatagtctactcttgt
gcagaatgaattctcgtaactacatagcacaagtagatgtagtta
actttaatctcacatagcaatctttaatcagtgtgtaacattagg
gaggacttgaaagagccaccacattttcaccgaggccacgcggag
tacgatcgagtgtacagtgaacaatgctagggagagctgcctata
tggaagagccctaatgtgtaaaattaattttagtagtgctatccc
catgtgattttaatagcttcttaggagaatgacaaaaaaaaaaaa
aaaaaaaaaaaaaaaaaaaaa.

1. Spike Protein

Aspects of the disclosure are related to a coronavirus spike protein. In some embodiments, the spike protein is SARS-CoV-2 Spike protein. SARS-CoV-2 Spike (or “S”) protein (also “S glycoprotein” or “Spike”), is involved in viral entry of SARS-CoV-2 via binding to angiotensin-converting enzyme 2 (ACE2) on a host cell. SARS-CoV-2 Spike protein is represented by Uniprot® Accession number P0DTC2. The receptor-binding domain (RBD) of SARS-CoV-2 Spike protein directly binds to the peptidase domain of ACE2 and, without wishing to be bound by theory, is understood to comprise amino acid residues 319-541 of SARS-CoV-2 Spike protein. A region of the RBD, the receptor-binding motif (RBM), is the main functional motif in RBD and, without wishing to be bound by theory, is understood to comprise amino acid residues 437-508 of SARS-CoV-2 Spike protein. Sequences of RBD and RBM are provided in Table 1 below.

TABLE 1

| | | SEQ |
|---|---|---|
| | | ID |
| Region | Sequence | NO |
| SARS-COV-2 | RVQPTESIVRFPNITNLCPFGEVFN | 19 |
| RBD | ATRFASVYAWNRKRISNCVADYSVL | |
| | YNSASFSTFKCYGVSPTKLNDLCFT | |
| | NVYADSFVIRGDEVRQIAPGQTGKI | |
| | ADYNYKLPDDFTGCVIAWNSNNLDS | |
| | KVGGNYNYLYRLFRKSNLKPFERDI | |
| | STEIYQAGSTPCNGVEGFNCYFPLQ | |
| | SYGFQPTNGVGYQPYRVVVLSFELL | |
| | HAPATVCGPKKSTNLVKNKCVNF | |
| SARS-COV-2 | NSNNLDSKVGGNYNYLYRLFRKSNL | 20 |
| RBM | KPFERDISTEIYQAGSTPCNGVEGE | |
| | NCYFPLQSYGFQPTNGVGYQPY | |

2. SARS-CoV-2 Therapeutics

Aspects of the disclosure are related to methods and compositions for treatment of a SARS-CoV-2 infection. A “SARS-CoV-2 therapeutic” or “COVID-19 therapeutic,” as used herein, describes any composition useful in the treatment or prevention of a SARS-CoV-2 infection, including amelioration of one or more symptoms caused by or related to a SARS-CoV-2 infection. Various SARS-CoV-2 therapeutics are recognized herein including, for example, COVID-19 convalescent plasma, COVID-19 convalescent serum, anti-SARS-CoV-2 antibodies (e.g., anti-SARS-CoV-2 Spike protein antibodies, including anti-RBD antibodies), aptamers (e.g., aptamers that bind to SARS-CoV-2 Spike protein) anti-inflammatory agents, and anti-viral agents (e.g., inhibitors of viral replication such as remdesivir). One or more SARS-CoV-2 therapeutics may be excluded from embodiments of the disclosure.

Methods of the disclosure may comprise administration of one or more SARS-CoV-2 therapeutics to a subject having a SARS-CoV-2 infection, where a biological sample from the subject (e.g., blood sample, plasma sample, serum sample, etc.) was determined to comprise an anti-angiotensin II antibody (i.e., an antibody that binds to angiotensin II). Methods of the disclosure may comprise first detecting an anti-angiotensin II antibody in a biological sample from a subject then treating the subject with a SARS-CoV-2 therapeutic.

### II. Proteins

As used herein, a “protein” or “polypeptide” refers to a molecule comprising at least five amino acid residues. As used herein, the term “wild-type” refers to the endogenous version of a molecule that occurs naturally in an organism. In some embodiments, wild-type versions of a protein or polypeptide are employed, however, in many embodiments of the disclosure, a modified protein or polypeptide is employed to generate an immune response. The terms described above may be used interchangeably. A “modified protein” or “modified polypeptide” or a “variant” refers to a protein or polypeptide whose chemical structure, particularly its amino acid sequence, is altered with respect to the wild-type protein or polypeptide. In some embodiments, a modified/variant protein or polypeptide has at least one modified activity or function (recognizing that proteins or polypeptides may have multiple activities or functions). It is specifically contemplated that a modified/variant protein or polypeptide may be altered with respect to one activity or function yet retain a wild-type activity or function in other respects, such as immunogenicity.

Where a protein is specifically mentioned herein, it is in general a reference to a native (wild-type) or recombinant (modified) protein or, optionally, a protein in which any signal sequence has been removed. The protein may be isolated directly from the organism of which it is native, produced by recombinant DNA/exogenous expression methods, or produced by solid-phase peptide synthesis (SPPS) or other in vitro methods. In particular embodiments, there are isolated nucleic acid segments and recombinant vectors incorporating nucleic acid sequences that encode a polypeptide (e.g., an antibody or fragment thereof). The term “recombinant” may be used in conjunction with a polypeptide or the name of a specific polypeptide, and this generally refers to a polypeptide produced from a nucleic acid molecule that has been manipulated in vitro or that is a replication product of such a molecule.

In certain embodiments the size of a protein or polypeptide (wild-type or modified) may comprise, but is not limited to, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 110, 120, 130, 140, 150, 160, 170, 180, 190, 200, 210, 220, 230, 240, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1100, 1200, 1300, 1400, 1500, 1750, 2000, 2250, 2500 amino acid residues or greater, and any range derivable therein, or derivative of a corresponding amino sequence described or referenced herein. It is contemplated that polypeptides may be mutated by truncation, rendering them shorter than their corresponding wild-type form, also, they might be altered by fusing or conjugating a heterologous protein or polypeptide sequence with a particular function (e.g., for targeting or localization, for enhanced immunogenicity, for purification purposes, etc.). As used herein, the term “domain” refers to any distinct functional or structural unit of a protein or polypeptide, and generally refers to a sequence of amino acids with a structure or function recognizable by one skilled in the art.

The nucleotide as well as the protein, polypeptide, and peptide sequences for various genes have been previously disclosed, and may be found in the recognized computerized databases. Two commonly used databases are the National Center for Biotechnology Information's Genbank® and GenPept® databases (on the World Wide Web at ncbi.nlm.nih.gov/) and The Universal Protein Resource (UniProt®; on the World Wide Web at uniprot.org). The coding regions for these genes may be amplified and/or expressed using the techniques disclosed herein or as would be known to those of ordinary skill in the art.

It is contemplated that in compositions of the disclosure, there may be between about 0.001 mg and about 10 mg of total polypeptide, peptide, and/or protein per ml. The concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein).

A. Angiotensin II

Aspects of the disclosure relate to the polypeptide Angiotensin II (AngII). AngII is a vasoconstrictor peptide and, without wishing to be bound by theory, is understood to be enzymatically cleaved by angiotensin-converting enzyme (ACE2) into angiotensin (1-7), a vasodilator peptide. The sequence of AngII is DRVYIHPF (SEQ ID NO:22). One of the key AngII cognate receptors is Type-1 angiotensin II receptor (AT1), which is involved in the physiological regulation of vasoconstriction and inflammation, as well as in multiple diseases, including hypertension, diabetes mellitus and renal end-stage disease (Iwai and Horiuchi, Hypertension Research (2009) 32, 533-536, incorporated herein by reference in its entirety).

### III. Pharmaceutical Compositions

The present disclosure includes methods for treating disease and modulating immune responses in a subject in need thereof. The disclosure includes cells that may be in the form of a pharmaceutical composition that can be used to induce or modify an immune response.

Administration of the compositions according to the current disclosure will typically be via any common route. This includes, but is not limited to parenteral, orthotopic, intradermal, subcutaneous, orally, transdermally, intramuscular, intraperitoneal, intraperitoneally, intraorbitally, by implantation, by inhalation, intraventricularly, intranasally or intravenous injection. In some embodiments, compositions of the present disclosure (e.g., vaccine compositions) are administered to a subject intravenously.

Typically, compositions and therapies of the disclosure are administered in a manner compatible with the dosage formulation, and in such amount as will be therapeutically effective and immune modifying. The quantity to be administered depends on the subject to be treated. Precise amounts of active ingredient required to be administered depend on the judgment of the practitioner.

The manner of application may be varied widely. Any of the conventional methods for administration of pharmaceutical compositions comprising cellular components are applicable. The dosage of the pharmaceutical composition will depend on the route of administration and will vary according to the size and health of the subject.

In many instances, it will be desirable to have multiple administrations of at most or at least 3, 4, 5, 6, 7, 8, 9, 10 or more. The administrations may range from 2-day to 12-week intervals, more usually from one to two week intervals.

The phrases “pharmaceutically acceptable” or “pharmacologically acceptable” refer to molecular entities and compositions that do not produce an adverse, allergic, or other untoward reaction when administered to an animal, or human. As used herein, “pharmaceutically acceptable carrier” includes any and all solvents, dispersion media, coatings, antibacterial and antifungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. The pharmaceutical compositions of the current disclosure are pharmaceutically acceptable compositions.

The compositions of the disclosure can be formulated for parenteral administration, e.g., formulated for injection via the intravenous, intramuscular, sub-cutaneous, or even intraperitoneal routes. Typically, such compositions can be prepared as injectables, either as liquid solutions or suspensions and the preparations can also be emulsified.

Pharmaceutical forms suitable for injectable use include sterile aqueous solutions or dispersions; formulations including sesame oil, peanut oil, or aqueous propylene glycol. It also should be stable under the conditions of manufacture and storage and must be preserved against the contaminating action of microorganisms, such as bacteria and fungi.

Sterile injectable solutions are prepared by incorporating the active ingredients (e.g., polypeptides of the disclosure) in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

An effective amount of a composition is determined based on the intended goal. The term “unit dose” or “dosage” refers to physically discrete units suitable for use in a subject, each unit containing a predetermined quantity of the composition calculated to produce the desired responses discussed herein in association with its administration, i.e., the appropriate route and regimen. The quantity to be administered, both according to number of treatments and unit dose, depends on the result and/or protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition. Upon formulation, solutions will be administered in a manner compatible with the dosage formulation and in such amount as is therapeutically or prophylactically effective. The formulations are easily administered in a variety of dosage forms, such as the type of injectable solutions described above.

The compositions and related methods of the present disclosure, particularly administration of a composition of the disclosure may also be used in combination with the administration of additional therapies such as the additional therapeutics described herein or in combination with other traditional therapeutics known in the art.

The therapeutic compositions and treatments disclosed herein may precede, be co-current with and/or follow another treatment or agent by intervals ranging from minutes to weeks. In embodiments where agents are applied separately to a cell, tissue or organism, one would generally ensure that a significant period of time did not expire between the time of each delivery, such that the therapeutic agents would still be able to exert an advantageously combined effect on the cell, tissue or organism. For example, in such instances, it is contemplated that one may contact the cell, tissue or organism with two, three, four or more agents or treatments substantially simultaneously (i.e., within less than about a minute). In other aspects, one or more therapeutic agents or treatments may be administered or provided within 1 minute, 5 minutes, 10 minutes, 20 minutes, 30 minutes, 45 minutes, 60 minutes, 2 hours, 3 hours, 4 hours, 5 hours, 6 hours, 7 hours, 8 hours, 9 hours, 10 hours, 11 hours, 12 hours, 13 hours, 14 hours, 15 hours, 16 hours, 17 hours, 18 hours, 19 hours, 20 hours, 21 hours, 22 hours, 22 hours, 23 hours, 24 hours, 25 hours, 26 hours, 27 hours, 28 hours, 29 hours, 30 hours, 31 hours, 32 hours, 33 hours, 34 hours, 35 hours, 36 hours, 37 hours, 38 hours, 39 hours, 40 hours, 41 hours, 42 hours, 43 hours, 44 hours, 45 hours, 46 hours, 47 hours, 48 hours, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 1 week, 2 weeks, 3 weeks, 4 weeks, 5 weeks, 6 weeks, 7 weeks, or 8 weeks or more, and any range derivable therein, prior to and/or after administering another therapeutic agent or treatment.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In some embodiments, the therapeutically effective or sufficient amount of the immune checkpoint inhibitor, such as an antibody and/or microbial modulator, that is administered to a human will be in the range of about 0.01 to about 50 mg/kg of patient body weight whether by one or more administrations. In some embodiments, the therapy used is about 0.01 to about 45 mg/kg, about 0.01 to about 40 mg/kg, about 0.01 to about 35 mg/kg, about 0.01 to about 30 mg/kg, about 0.01 to about 25 mg/kg, about 0.01 to about 20 mg/kg, about 0.01 to about 15 mg/kg, about 0.01 to about 10 mg/kg, about 0.01 to about 5 mg/kg, or about 0.01 to about 1 mg/kg administered daily, for example. In one embodiment, a therapy described herein is administered to a subject at a dose of about 100 mg, about 200 mg, about 300 mg, about 400 mg, about 500 mg, about 600 mg, about 700 mg, about 800 mg, about 900 mg, about 1000 mg, about 1100 mg, about 1200 mg, about 1300 mg or about 1400 mg on day 1 of 21-day cycles. The dose may be administered as a single dose or as multiple doses (e.g., 2 or 3 doses), such as infusions. The progress of this therapy is easily monitored by conventional techniques.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

A. Vaccine Compositions

Aspects of the present disclosure are directed to vaccine compositions (i.e., compositions suitable for use in vaccination of a subject). A vaccine composition may be a composition suitable for treatment or prevention of a viral infection. Vaccine compositions include, for example, compositions comprising a viral polypeptide or a nucleic acid encoding for a viral polypeptide. In some embodiments, disclosed are vaccine compositions for treating or preventing COVID-19 in a subject. In some embodiments, such vaccine compositions comprise a SARS-CoV-2 protein, or portion thereof. In some embodiments, such vaccine compositions comprise a nucleic acid (e.g., mRNA, DNA) encoding for a SARS-CoV-2 protein, or for a portion of a SARS-CoV-2 protein. In some embodiments, the SARS-CoV-2 protein is SARS-CoV-2 Spike protein. In some embodiments, a vaccine composition of the disclosure comprises the RBD region of SARS-CoV-2 Spike protein. In some embodiments, a vaccine composition of the disclosure comprises a nucleic acid encoding for the RBD region of SARS-CoV-2 Spike protein. In some embodiments, a vaccine composition of the disclosure comprises the RBM region of SARS-CoV-2 Spike protein. In some embodiments, a vaccine composition of the disclosure comprises a nucleic acid encoding for the RBM region of SARS-CoV-2 Spike protein.

The immunogenicity of a particular vaccine composition can be enhanced by the use of non-specific stimulators of the immune response, known as adjuvants. Adjuvants that may be used in accordance with embodiments include, but are not limited to, IL-1, IL-2, IL-4, IL-7, IL-12, γ-interferon, GM-CSF, BCG, aluminum hydroxide, MDP compounds, such as thur-MDP and nor-MDP, CGP (MTP-PE), lipid A, and monophosphoryl lipid A (MPL). Exemplary adjuvants may include complete Freund's adjuvant (a non-specific stimulator of the immune response containing killed Mycobacterium tuberculosis), incomplete Freund's adjuvants, Matrix-M™ Adjuvant, AS03, AS04, CpG, poly(I:C), imidazoquinoline adjuvants, and/or aluminum hydroxide adjuvant. In some embodiments, a vaccine composition of the disclosure comprises an adjuvant. In some embodiments, a vaccine composition of the disclosure does not comprise an adjuvant.

In addition to adjuvants, it may be desirable to co-administer biologic response modifiers (BRM), such as but not limited to, Cimetidine (CIM; 1200 mg/d) (Smith/Kline, PA); low-dose Cyclophosphamide (CYP; 300 mg/m²) (Johnson/Mead, NJ), cytokines such as β-interferon, IL-2, or IL-12, or genes encoding proteins involved in immune helper functions, such as B-7.

### IV. Sample Preparation

In certain aspects, methods involve obtaining a sample (also “biological sample”) from a subject. The methods of obtaining provided herein may include methods of biopsy such as fine needle aspiration, core needle biopsy, vacuum assisted biopsy, incisional biopsy, excisional biopsy, punch biopsy, shave biopsy or skin biopsy. In some embodiments the sample may be obtained from any of the tissues provided herein that include but are not limited to non-cancerous or cancerous tissue and non-cancerous or cancerous tissue from the serum, gall bladder, mucosal, skin, heart, lung, breast, pancreas, blood, liver, muscle, kidney, smooth muscle, bladder, colon, intestine, brain, prostate, esophagus, or thyroid tissue. Alternatively, the sample may be obtained from any other source including but not limited to blood, plasma, serum, sweat, hair follicle, buccal tissue, tears, menses, feces, or saliva. In some embodiments, the sample is a blood sample. In some embodiments, the sample is a serum sample. In some embodiments, the sample is a plasma sample. In certain aspects of the current methods, any medical professional such as a doctor, nurse or medical technician may obtain a biological sample for testing. Yet further, the biological sample can be obtained without the assistance of a medical professional.

A sample may include but is not limited to, tissue, cells, or biological material from cells or derived from cells of a subject. The biological sample may be a heterogeneous or homogeneous population of cells or tissues. Alternatively, the sample may be a cell-free sample. The biological sample may be obtained using any method known to the art that can provide a sample suitable for the analytical methods described herein. The sample may be obtained by non-invasive methods including but not limited to: scraping of the skin or cervix, swabbing of the cheek, saliva collection, urine collection, feces collection, collection of menses, tears, or semen.

The sample may be obtained by methods known in the art. In certain embodiments the samples are obtained by biopsy. In other embodiments the sample is obtained by swabbing, endoscopy, scraping, phlebotomy, or any other methods known in the art. In some cases, the sample may be obtained, stored, or transported using components of a kit of the present methods. In some cases, multiple samples, such as plasma samples, may be obtained for evaluation by the methods described herein (e.g., identification of antibodies that bind AngII).

In some embodiments the biological sample may be obtained by a physician, nurse, or other medical professional such as a medical technician, endocrinologist, cytologist, phlebotomist, radiologist, or a pulmonologist. The medical professional may indicate the appropriate test or assay to perform on the sample. In certain aspects a molecular profiling business may consult on which assays or tests are most appropriately indicated. In further aspects of the current methods, the patient or subject may obtain a biological sample for testing without the assistance of a medical professional, such as obtaining a whole blood sample, a urine sample, a fecal sample, a buccal sample, or a saliva sample.

In other cases, the sample is obtained by an invasive procedure including but not limited to: biopsy, needle aspiration, endoscopy, or phlebotomy. The method of needle aspiration may further include fine needle aspiration, core needle biopsy, vacuum assisted biopsy, or large core biopsy. In some embodiments, multiple samples may be obtained by the methods herein to ensure a sufficient amount of biological material.

General methods for obtaining biological samples are also known in the art. Publications such as Ramzy, Ibrahim Clinical Cytopathology and Aspiration Biopsy 2001, which is herein incorporated by reference in its entirety, describes general methods for biopsy and cytological methods. In one embodiment, the sample is a fine needle aspirate of a esophageal or a suspected esophageal tumor or neoplasm. In some cases, the fine needle aspirate sampling procedure may be guided by the use of an ultrasound, X-ray, or other imaging device.

In some embodiments of the present methods, the molecular profiling business may obtain the biological sample from a subject directly, from a medical professional, from a third party, or from a kit provided by a molecular profiling business or a third party. In some cases, the biological sample may be obtained by the molecular profiling business after the subject, a medical professional, or a third party acquires and sends the biological sample to the molecular profiling business. In some cases, the molecular profiling business may provide suitable containers, and excipients for storage and transport of the biological sample to the molecular profiling business.

In some embodiments of the methods described herein, a medical professional need not be involved in the initial diagnosis or sample acquisition. An individual may alternatively obtain a sample through the use of an over the counter (OTC) kit. An OTC kit may contain a means for obtaining said sample as described herein, a means for storing said sample for inspection, and instructions for proper use of the kit. In some cases, molecular profiling services are included in the price for purchase of the kit. In other cases, the molecular profiling services are billed separately. A sample suitable for use by the molecular profiling business may be any material containing tissues, cells, nucleic acids, genes, gene fragments, expression products, gene expression products, or gene expression product fragments of an individual to be tested. Methods for determining sample suitability and/or adequacy are provided.

In some embodiments, the subject may be referred to a specialist such as an oncologist, surgeon, or endocrinologist. The specialist may likewise obtain a biological sample for testing or refer the individual to a testing center or laboratory for submission of the biological sample. In some cases the medical professional may refer the subject to a testing center or laboratory for submission of the biological sample. In other cases, the subject may provide the sample. In some cases, a molecular profiling business may obtain the sample.

In some embodiments, a sample is a convalescent sample (i.e., a sample from a patient who has recovered from a disease or infection). In some embodiments, a sample is a convalescent sample from a subject who has recovered from a coronavirus infection. In some embodiments, a sample is a convalescent sample from a subject who has had a SARS-CoV-2 infection. In some embodiments, a convalescent sample is a convalescent plasma sample.

### EXAMPLES

The following examples are included to demonstrate certain embodiments of the invention. It should be appreciated by those of skill in the art that the techniques disclosed in the examples which follow represent techniques discovered by the inventor to function well in the practice of the invention, and thus can be considered to constitute certain modes for its practice. However, those of skill in the art should, in light of the present disclosure, appreciate that many changes can be made in the specific embodiments which are disclosed and still obtain a like or similar result without departing from the spirit and scope of the invention.

### Example 1—Detection of Anti-AngII Antibodies in SARS-CoV-2 Patients

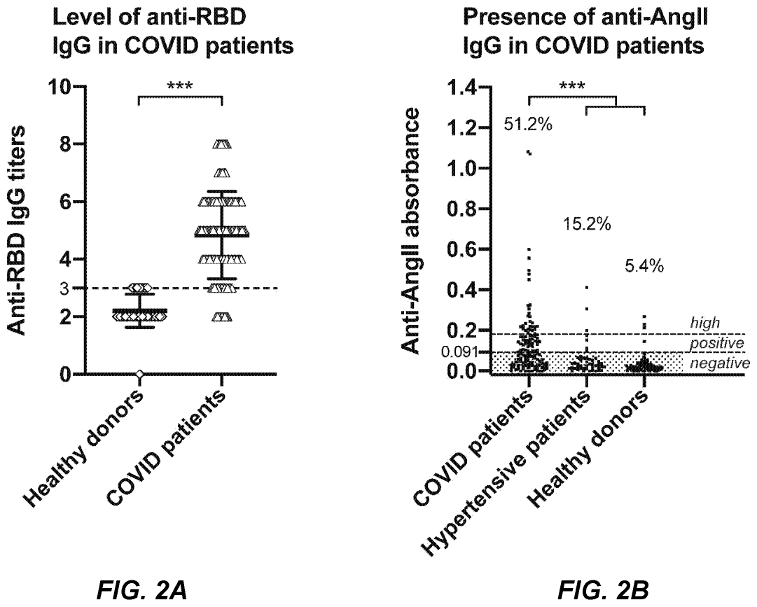

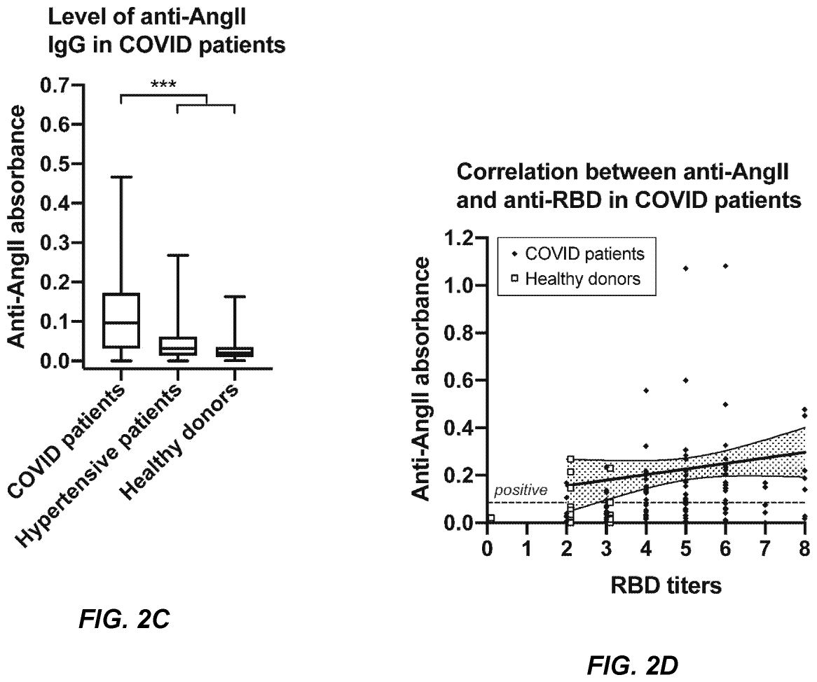

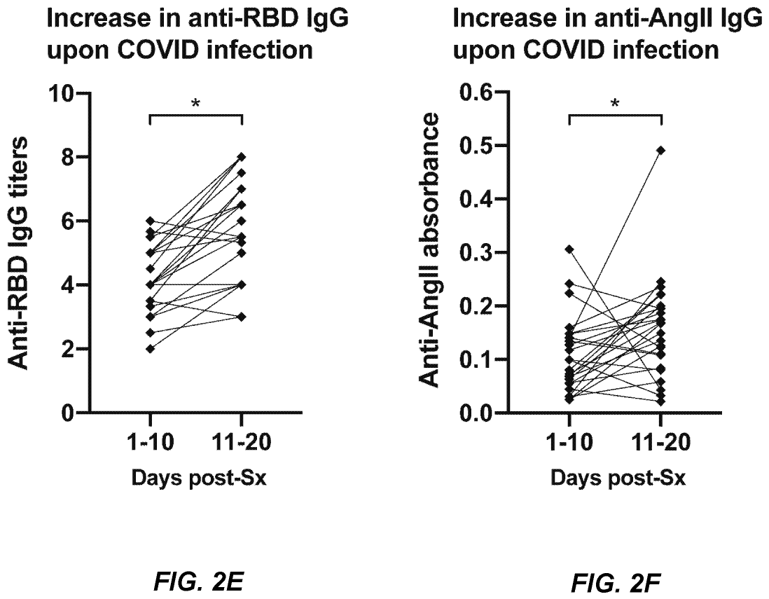

The presence of anti-AngII IgG auto-antibodies was assessed in the plasma/serum of 247 subjects, amongst which 127 were patients with COVID-19 (i.e., SARS-CoV-2) infection or were convalescent, 74 were healthy donors (i.e. non-SARS-CoV-2 infected), and 46 were hypertensive non-SARS-CoV-2 infected donors. Most COVID-19 patients still had detectable anti-RBD antibody titers at the time of analysis, here considered as above an IgG titer threshold of 3 (FIG. 2A). The inventors found that among the COVID-19 patients, 51.2% (65 of 127) had detectable levels of anti-AngII autoantibodies, as determined by an ELISA absorbance greater than 3 standard deviations above the mean in control (non-COVID-19 healthy donor) serum (after removal of outliers according to the IQR rule). Of these, 23.6% (30 of 127) had high levels, defined as an absorbance greater than twice the positive threshold (FIG. 2B). These results indicated that infection with SARS-CoV-2 promotes the development of anti-AngII antibodies in a subpopulation of patients. Indeed, a significantly lower proportion of patients were found to be anti-AngII positive in the healthy donors cohort and in the non-COVID-19 hypertensive cohort, which respectively had 5.4% (4 of 74) and 15.2% (7 of 46). In addition to the proportion of anti-AngII positive patients, the amount of anti-AngII autoantibodies was significantly increased in the COVID-19 patient cohort as compared to the healthy or the non-COVID-19 hypertensive donors (FIG. 2C).

In the subset of anti-AngII positive COVID patients (65 patients), the levels of anti-AngII autoantibodies had a low but significant correlation with the levels of anti-RBD antibodies (Spearman correlation test, R=0.295, p-value=0.0172), highlighted in FIG. 2D. Furthermore, 25 of these patients were analyzed for anti-RBD and anti-AngII levels at multiple time points upon COVID-19 infection. The inventors found a significant increase of anti-RBD IgG titers between 1-10 and 11-20 days (FIG. 2E), as well as a significant increase of anti-AngII levels over the same times (FIG. 2F). These results suggest that anti-RBD and anti-AngII IgG antibodies may be conjointly raised in these patients.

### Methods

All samples collection and data recording on human patients and donors were approved by the IRB, under an informed consent, from a cohort at the University of Chicago. Patients of the COVID cohort were hospitalized patients, in contrast to non-SARS-CoV-2 infected healthy and hypertensive individuals, which were non-hospitalized donors. In addition, many of the COVID-19 patients had pre-existing health conditions, including hypertension. Blood was analyzed for presence and amounts of specific anti-RBD IgG and anti-AngII IgG antibodies by ELISA.

All procedures utilizing human samples were performed under Biosafety level 2 conditions. For the titration of anti-RBD antibodies, enzyme-linked immunosorbent assay (ELISA) plates were coated with purified recombinant RBD (100 nM) in PBS for 12-16 hours at 4° C. Plates were then blocked with bovine serum albumin (BSA, 2%) in PBS for 2 hours at room temperature. Plates were then extensively washed with PBS-Tween 0.05% (PBST). Human plasmas/sera were diluted at 1:100 in PBST+BSA (0.5%), and then serially diluted by 10. Samples were added to the plates and incubated for 2 hours at room temperature. Plates were washed again, and incubated with an horseradish peroxidase (HRP)-conjugated anti-human IgG for 1 hour at room temperature in the dark. Plates were washed, revealed using 3,3′,5,5′-Tetramethylbenzidine (TMB) for 30 min in the dark, and the reaction was stopped using 1M H₂SO₄. Absorbances at 450 nm were read using an Epoch plate reader (BioTek), and corrected with the absorbance at 570 nm. Titers represent the highest dilution at which antibodies were detected, in log 10 (e.g. an IgG titer of 4 indicates a detectable level of anti-RBD IgG in a plasma/serum sample diluted at 1:10000). A value of 0 was given to samples that had no detectable IgG at a dilution of 1:100.

For the detection of anti-AngII antibodies, streptavidin-coated plates were purchased from Pierce (ThermoFisher Scientific) and biotin-LC-AngII was purchased from AnaSpec. Streptavidin-coated plates were re-hydrated using PBST, and biotin-LC-AngII (10 μg/mL) was added to the plate for 2 hours at room temperature. Plates were washed with PBST, and plasma/sera samples diluted at 1:100 were added to the plates for 2 hours at room temperature. Plates were washed again and incubated with an HRP-conjugated anti-human IgG for 1 hour at room temperature in the dark. Plates were washed and developed as previously described, except that the signals were stopped after about 10-15 min. Absorbances at 450 nm were corrected with the absorbance at 570 nm. Detection of antibodies in absence of biotin-LC-AngII was also performed and subtracted to correct for AngII-unspecific antibody binding.

The ectodomain of Spike (SEQ ID NO:17) or the subdomain RBD (SEQ ID NO:18) were cloned into pCAGGS vector for mammalian expression in human embryonic kidney (HEK) 293F suspension cells, as his-tagged recombinant proteins. The proteins were expressed for 4-7 days in FreeStyle medium at 37° C., 5% CO₂, under constant agitation. Cell supernatants were then collected and filtered at 0.22 μm. The proteins were purified by immobilized nickel-affinity chromatography (HisTrap HP column, GE Healthcare) using fast protein liquid chromatography (FPLC; Äkta Pure system, GE Healthcare). Some protein batches were additionally purified by size exclusion chromatography (Superdex 200 pg column, GE Healthcare), and/or added with dithiothreitol (DTT) 10 mM to reduce dimerization. Proteins were then extensively dialyzed against phosphate-buffered saline (PBS; pH 7.4), sterile-filtered and stored at −80° C. All proteins were tested for endotoxin level <2 EU/mg.

TABLE 2
Sequences

| | | SEQ |
|---|---|---|
| | | ID |
| Protein | Sequence | NO |
| SARS-COV-2 | MFVFLVLLPLVSSQCVNLTTRTQLP | 17 |
| Spike ectodomain | PAYTNSFTRGVYYPDKVFRSSVLHS | |
| (with His tag) | TQDLFLPFFSNVTWFHAIHVSGTNG | |
| | TKRFDNPVLPFNDGVYFASTEKSNI | |
| | IRGWIFGTTLDSKTQSLLIVNNATN | |
| | VVIKVCEFQFCNDPFLGVYYHKNNK | |
| | SWMESEFRVYSSANNCTFEYVSQPF | |
| | LMDLEGKQGNFKNLREFVFKNIDGY | |
| | FKIYSKHTPINLVRDLPQGFSALEP | |
| | LVDLPIGINITRFQTLLALHRSYLT | |
| | PGDSSSGWTAGAAAYYVGYLQPRTF | |
| | LLKYNENGTITDAVDCALDPLSETK | |
| | CTLKSFTVEKGIYQTSNFRVQPTES | |
| | IVRFPNITNLCPFGEVFNATRFASV | |
| | YAWNRKRISNCVADYSVLYNSASFS | |
| | TFKCYGVSPTKLNDLCFTNVYADSF | |
| | VIRGDEVRQIAPGQTGKIADYNYKL | |
| | PDDFTGCVIAWNSNNLDSKVGGNYN | |
| | YLYRLFRKSNLKPFERDISTEIYQA | |
| | GSTPCNGVEGENCYFPLQSYGFQPT | |
| | NGVGYQPYRVVVLSFELLHAPATVC | |
| | GPKKSTNLVKNKCVNFNFNGLTGTG | |
| | VLTESNKKFLPFQQFGRDIADTTDA | |
| | VRDPQTLEILDITPCSFGGVSVITP | |
| | GTNTSNQVAVLYQDVNCTEVPVAIH | |
| | ADQLTPTWRVYSTGSNVFQTRAGCL | |
| | IGAEHVNNSYECDIPIGAGICASYQ | |
| | TQTNSPASVASQSIIAYTMSLGAEN | |
| | SVAYSNNSIAIPTNFTISVTTEILP | |
| | VSMTKTSVDCTMYICGDSTECSNLL | |
| | LQYGSFCTQLNRALTGIAVEQDKNT | |
| | QEVFAQVKQIYKTPPIKDFGGFNFS | |
| | QILPDPSKPSKRSFIEDLLFNKVTL | |
| | ADAGFIKQYGDCLGDIAARDLICAQ | |
| | KENGLTVLPPLLTDEMIAQYTSALL | |
| | AGTITSGWTFGAGAALQIPFAMQMA | |
| | YRFNGIGVTQNVLYENQKLIANQFN | |
| | SAIGKIQDSLSSTASALGKLQDVVN | |
| | QNAQALNTLVKQLSSNFGAISSVLN | |
| | DILSRLDPPEAEVQIDRLITGRLQS | |
| | LQTYVTQQLIRAAEIRASANLAATK | |
| | MSECVLGQSKRVDFCGKGYHLMSFP | |
| | QSAPHGVVFLHVTYVPAQEKNFTTA | |
| | PAICHDGKAHFPREGVFVSNGTHWF | |
| | VTQRNFYEPQIITTDNTFVSGNCDV | |
| | VIGIVNNTVYDPLQPELDSFKEELD | |
| | KYFKNHTSPDVDLGDISGINASVVN | |
| | IQKEIDRLNEVAKNLNESLIDLQEL | |
| | GKYEQYIKWPSGRLVPRGSPGSGYI | |
| | PEAPRDGQAYVRKDGEWVLLSTFLG | |
| | HHHHHH | |
| SARS-COV-2 RBD | MFVFLVLLPLVSSQRVQPTESIVRF | |
| sequence | PNITNLCPFGEVENATRFASVYAWN | |
| (with His | RKRISNCVADYSVLYNSASFSTFKC | |
| tag) | YGVSPTKLNDLCFTNVYADSFVIRG | |
| | DEVRQIAPGQTGKIADYNYKLPDDF | |
| | TGCVIAWNSNNLDSKVGGNYNYLYR | |
| | LFRKSNLKPFERDISTEIYQAGSTP | 18 |
| | CNGVEGENCYFPLQSYGFQPTNGVG | |
| | YQPYRVVVLSFELLHAPATVCGPKK | |
| | STNLVKNKCVNFHHHHHH | |

Streptavidin-coated plates were purchased from Pierce (ThermoFisher Scientific) and biotin-LC-AngII was purchased from AnaSpec. Streptavidin-coated plates were re-hydrated using PBST, and biotin-LC-AngII (10 μg/mL) was added to the plate for 2 hours at room temperature. Plates were washed with PBST, and plasma/sera samples diluted at 1:100 were added to the plates for 2 hours at room temperature. Plates were washed again and incubated with an HRP-conjugated anti-mouse IgG (1:8000; Southern Biotech) or anti-human IgG for 1 hour at room temperature in the dark. Plates were washed and developed as described above. Absorbances at 450 nm were corrected with the absorbance at 570 nm. Detection of antibodies in absence of biotin-LC-AngII was also performed and subtracted to correct for AngII-unspecific antibody binding.

### Example 2—Vaccination Against SARS-CoV-2 can Raise Anti-AngII Autoantibodies in Mice

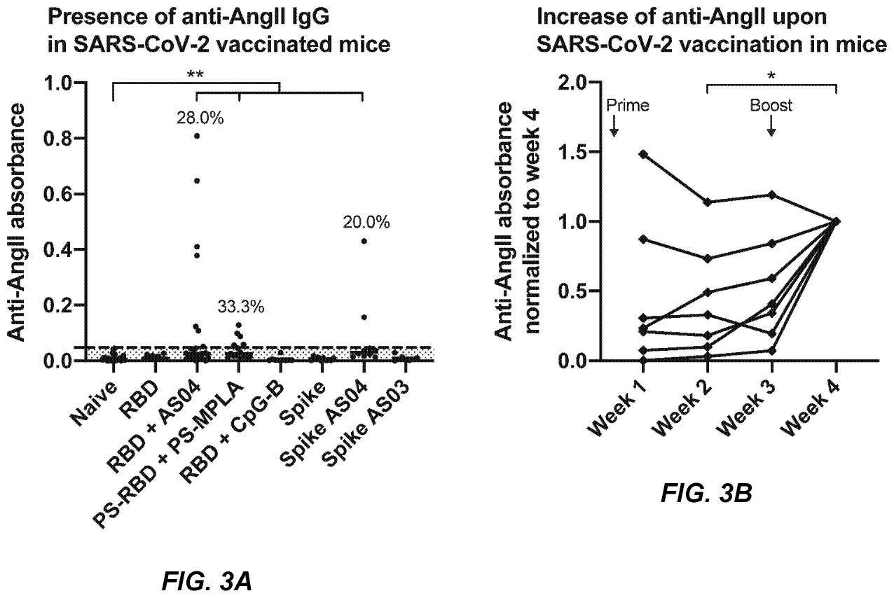

To determine whether the development of anti-AngII antibodies is induced by exposure to SARS-CoV-2 antigens, the inventors vaccinated mice with adjuvanted formulations of SARS-CoV-2 envelope antigens, namely Spike and RBD. Mice were vaccinated at week 0 and 3, with 10 μg of the antigen admixed with an adjuvant mimicking the adjuvant AS04 (a formulation of the TLR4 agonist monophosphoryl lipid A (MPLA) in alum), an adjuvant mimicking AS03 (AddaS03™; a formulation of squalene and polysorbate 80 in an oil-in-water nano-emulsion), the TLR9 agonist CpG-B, and a polymersome formulation in which MPLA was formulated in polymersomes (PS-MPLA) and RBD was encapsulated within polymersomes (PS-RBD), or with no adjuvants. The mice plasma was analyzed at week 4. All mice vaccinated with the adjuvanted formulations developed high levels of anti-RBD antibodies at the time of analysis (titers ≥4), as compared to non-adjuvanted formulations that induced low anti-RBD titers (titers ≥2), and that naïve mice had undetectable levels of anti-RBD. As to anti-AngII development, the inventors used a threshold of mean plus three standard deviations of the naïve group to determine a positive response. None of the naïve mice (0 of 25) had detectable anti-AngII IgG. 28.0% (7 of 25) of the mice vaccinated with RBD+AS04, 33.3% (5 of 15) of the mice vaccinated with PS-RBD+PS-MPLA, and 20% (2 of 10) of mice vaccinated with Spike+AS04 developed antibodies against AngII (FIG. 3A). The formulation with AS04 induced higher levels of anti-AngII than the polymersome formulation with MPLA. In contrast, none of the mice vaccinated with RBD+CpG (0 of 10) or with RBD+AS03 (0 of 5) developed anti-AngII antibodies, and no mice vaccinated with unadjuvanted RBD (0 of 10) or unadjuvanted Spike (0 of 10) developed anti-AngII antibodies. These results demonstrated that anti-AngII autoantibodies can be raised in response to the host immune reaction against SARS-CoV-2 Spike/RBD antigens and pinpointed a potential adverse effect of SARS-CoV-2 vaccination in healthy organisms.

Furthermore, the levels of anti-AngII antibodies were analyzed at weeks 1, 2, 3 and 4 in the 7 mice vaccinated with RBD+AS04 that developed anti-AngII antibodies. A significant increase was observed in anti-AngII levels between week 2 and 4 upon vaccination (FIG. 3B). This raise in anti-AngII IgG seemed to be more important after the vaccination boost at week 3.

### Methods

Vaccines were formulated in PBS, by mixing 10 μg SARS-CoV-2 antigens, i.e. RBD or Spike, with various adjuvants. AS04 was prepared by mixing 5 μg of MPLA (InvivoGen) with 50 μg of Alum (InvivoGen). AS03 was prepared by mixing 25 μL of AddaS03™ (Invivogen) with 25 μL of PBS containing the SARS-CoV-2 antigens. Mouse specific stimulatory CpG class B (ODN1826; InvivoGen) was injected at a dose of 20 μg. In one group, the antigen RBD and adjuvant MPLA were formulated into self-assembled poly(ethylene glycol)-co-poly(propylene sulfide) (PEG-PPS) polymersomes (PS). PS-RBD was injected at a dose equivalent of 10 μg of RBD, and PS-MPLA a dose equivalent to 5 μg MPLA. Non-adjuvanted groups were composed of 10 μg of antigen only.

All mouse experimentation was approved by the IACUC. Female 8-week old C57BL/6 mice received a prime vaccination using the vaccine formulations above-described, as well as a boost vaccination 3 weeks later. Vaccinations were administered by intradermal injections in the animal hocks in the two forelimbs (25 μL/hock). Mice were bled weekly via the submandibular vein after vaccination for plasma collection and analysis. Anti-AngII IgG levels were determined as described above, except using an anti-mouse total IgG as a detection antibody (1:8000; Southern Biotech).

### Example 3—Cross-Binding of a Monoclonal Anti-AngII to SARS-CoV-2 Spike and RBD

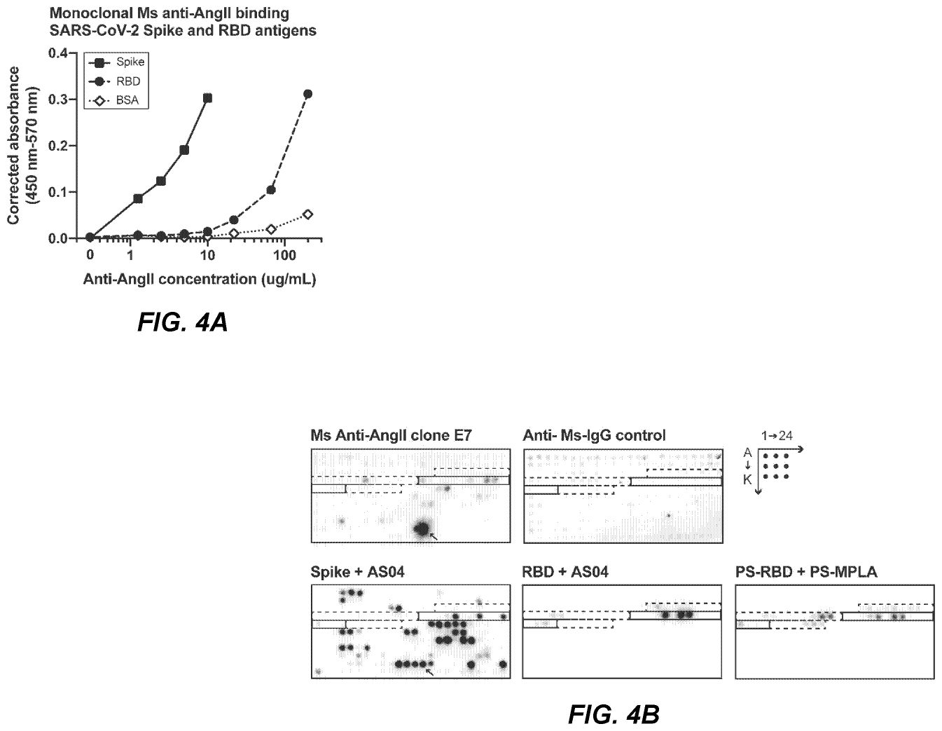

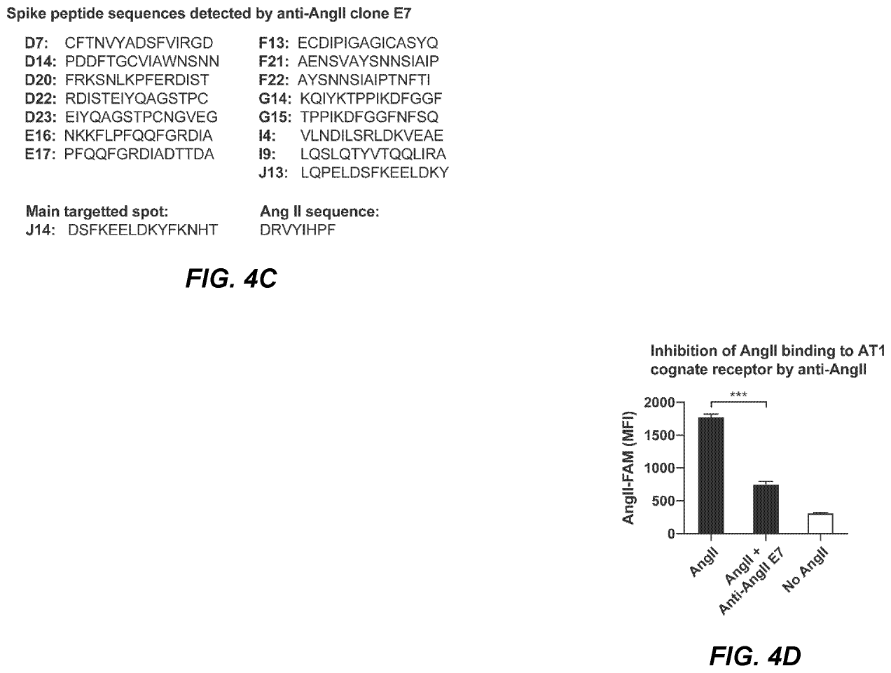

The inventors questioned whether the development of anti-AngII might result from a molecular structural mimetism between the AngII peptide and certain epitopes present in Spike or RBD. In such a case, anti-AngII and anti-RBD might cross-bind to both antigen molecules. To test this hypothesis, the inventors assessed the binding of a mouse IgG2a monoclonal anti-AngII/III antibody (clone E7; MA1-82996) to recombinant SARS-CoV-2 Spike or RBD (FIG. 4A). The monoclonal anti-AngII E7 bound to both Spike and RBD, although with a lower affinity to RBD than to Spike. The Spike linear epitopes that are targeted by the anti-AngII E7 antibody were then mapped using a peptide array. This array contains a collection of 15-mers peptides, shifted by 5 amino acids, covering the full length of Spike. Binding of the anti-AngII E7 to the Spike-derived peptides was revealed using a secondary HRP-conjugated anti-mouse IgG antibody. As a control for nonspecific binding, the assay was repeated using the secondary antibody only (in absence of anti-AngII E7). The anti-AngII E7 bound to several linear epitopes on Spike, with a main target at the J14 position (see arrow, FIG. 4B), and about 15 secondary targets of lower affinity. Five of these secondary targeted peptides belong to the RBD of Spike, and 4 more particularly to the RBM of the RBD. The sequence of the peptides targeted by the anti-AngII E7 are detailed in FIG. 4C and Table 3.

TABLE 3

| Peptide | Sequence | SEQ ID NO |
|---|---|---|
| J14 | DSFKEELDKYFKNHT | 1 |
| D7 | CFTNVYADSFVIRGD | 2 |
| D14 | PDDFTGCVIAWNSNN | 3 |
| D20 | FRKSNLKPFERDIST | 4 |
| D22 | RDISTEIYQAGSTPC | 5 |
| D23 | EIYQAGSTPCNGVEG | 6 |
| E16 | NKKFLPFQQFGRDIA | 7 |
| E17 | PFQQFGRDIADTTDA | 8 |
| F13 | ECDIPIGAGICASYQ | 9 |
| F21 | AENSVAYSNNSIAIP | 10 |
| F22 | AYSNNSIAIPTNFTI | 11 |
| G14 | KQIYKTPPIKDFGGF | 12 |
| G15 | TPPIKDFGGFNFSQ | 13 |
| I4 | VLNDILSRLDKVEAE | 14 |
| 19 | LQSLQTYVTQQLIRA | 15 |
| J13 | LQPELDSFKEELDKY | 16 |

The inventors then compared this profile to the ones of the anti-RBD/Spike antibodies raised during mice vaccinations against the SARS-CoV-2 antigens. The peptide array assays were repeated using the sera of mice vaccinated with Spike+AS04, RBD+AS04 or PS-RBD+PS-MIPLA (pooled sera from 5 different mice), and revealed binding using the same anti-mouse IgG secondary antibody (FIG. 41B). The mice vaccinated with Spike+AS04 also developed antibodies that targeted the position J14 of the array, which was the primary target of anti-AngII E7. In addition, all 3 groups of vaccinated mice raised antibodies that targeted the RBM at the same or neighboring spots as the anti-AngII E7 monoclonal antibody.

Therefore, using the monoclonal anti-AngII E7 as a proof-of-concept, the inventors demonstrated that cross-binding of anti-AngII to SARS-CoV-2 Spike and RBD antigens can occur. These results suggested that there is a sufficient structural and/or sequence homology between AngII and some epitopes of Spike/RBD to allow antibody cross-reactivity.

Finally, the inventors showed that the anti-AngII E7 monoclonal antibody, which is cross-reactive to RBD/Spike, is capable of inhibiting AngII binding to its cognate receptor AT1, as highlighted by a significant reduction of the MFI of FAM-AngII in presence of the anti-AngII E7 antibody (FIG. 4D). Binding of AngII to AT1 is the initial step of AT1 signal transduction, which regulates important physiological mechanisms such as vasoconstriction. This result suggests that the presence of anti-AngII IgG in the serum/plasma of individuals could modulate endogenous binding of AngII to AT1, potentially affecting blood pressure regulation in these individuals.

### Methods

All procedures utilizing human samples were performed under Biosafety level 2 conditions. Enzyme-linked immunosorbent assay (ELISA) plates were coated with purified recombinant RBD (100 nM) in PBS for 12-16 hours at 4° C. Plates were then blocked with bovine serum albumin (BSA, 2%) in PBS for 2 hours at room temperature. Plates were then extensively washed with PBS-Tween 0.05% (PBST). Human plasmas/sera were diluted at 1:100 in PBST+BSA (0.5%), and then serially diluted by 10. Samples were added to the plates and incubated for 2 hours at room temperature. Plates were washed again, and incubated with an horseradish peroxidase (HRP)-conjugated anti-human IgG for 1 hour at room temperature in the dark. Plates were washed, revealed using 3,3′,5,5′-Tetramethylbenzidine (TMB) for 30 min in the dark, and the reaction was stopped using 1M H₂SO₄. Absorbances at 450 nm were read using a Epoch plate reader (BioTek), and corrected with the absorbance at 570 nm. Titration curves and half-maximum values were determined using Prism 8 (GraphPad).

A mouse IgG2a monoclonal anti-AngII/III clone E7 (MA1-82996) was purchased from ThermoFisher Scientific. ELISA plates were coated with RBD or Spike (100 nM) in PBS for 12-16 hours at 4° C. The plate was then blocked using BSA 2% for 2 hours at room temperature. The anti-AngII antibody was then diluted in PBST-BSA (0.5%) at the specified concentrations and added to the plate for 2 hours at room temperature. Binding of the anti-AngII to RBD, Spike or BSA was detected using an anti-mouse IgG (Southern Biotech) for 1 hour at room temperature in the dark. The plate was then revealed and the absorbance determined as described above.

SARS-CoV-2 Full Spike CelluSpots peptide array assays were purchased from Intavis Bioanalytical Instruments. The assay contains 254 peptides of 15 amino acids (aa) length covering the full-length Spike sequence with a shift of 5 aa between peptides. The assay was performed as instructed by the manufacturer. Briefly, the membrane was blocked with casein blocking buffer (Sigma-Aldrich) for 4 hours at room temperature, and then incubated for 12 hours at 4° C. with the anti-AngII clone E7 diluted at a concentration of 20 μg/mL in casein. The membrane was then washed extensively using PBST, and further incubated with an HRP-conjugated anti-mouse IgG (1:5000; Southern Biotech) for 2 hours at room temperature. The membrane was again extensively washed in PBST and revealed using the Clarity ECL Western substrate (BioRad). The membrane was imaged using a gel imager (BioRad).

Binding of AngII to AT1 receptors was assessed using fluorescently labelled FAM-AngII, purchased from AnaSpec, and Chinese Hamster Ovarian (CHO) cells expressing recombinant human AT1, purchased from Perkin-Elmer. FAM-AngII (100 nM) was pre-incubated with 0.3 mg/mL of the anti-AngII E7 monoclonal antibody in PBS for 30 min at 37° C. or with no antibody as a control. Then, CHO-AT1 cells were stained with these mixtures in the dark for 20 min on ice. Cells were washed 3 times in PBS+2% Fetal Bovine Serum, after which the binding of FAM-AngII to AT1 on the cell surface was detected using a flow cytometer (BD LSRFortessa, BD Biosciences). The mean fluorescence intensity (MFI) of the FAM-AngII was computed using FlowJo software.

### Example 4—Presence of Anti-AngII IgG Correlates with Increased Clinical Severity in Hospitalized COVID Patients, Particularly Those with Pre-Existing Hypertension

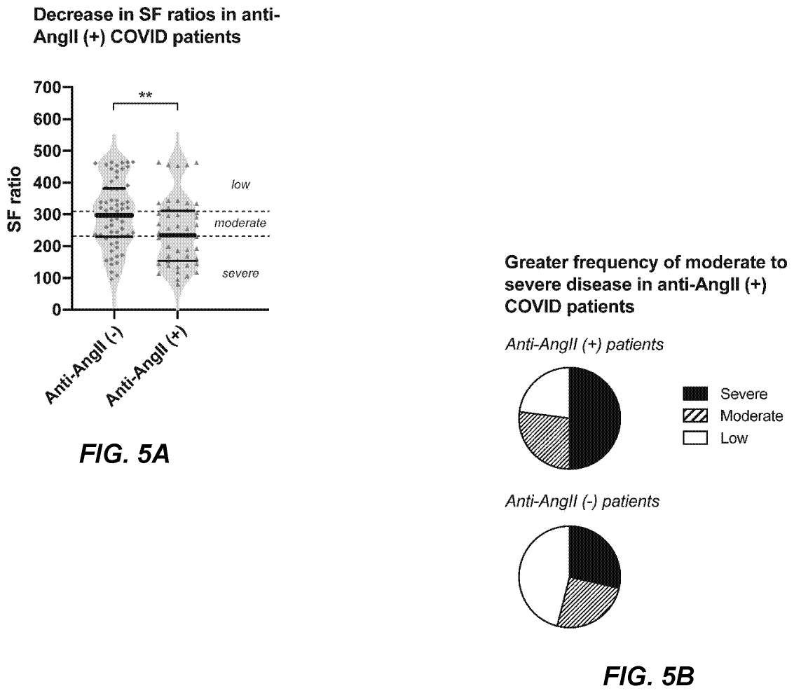

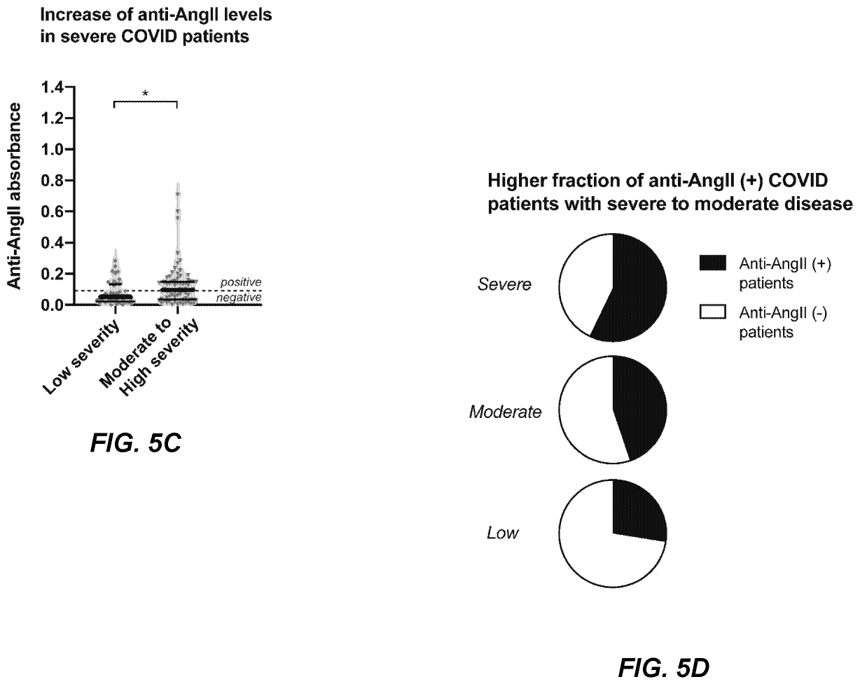

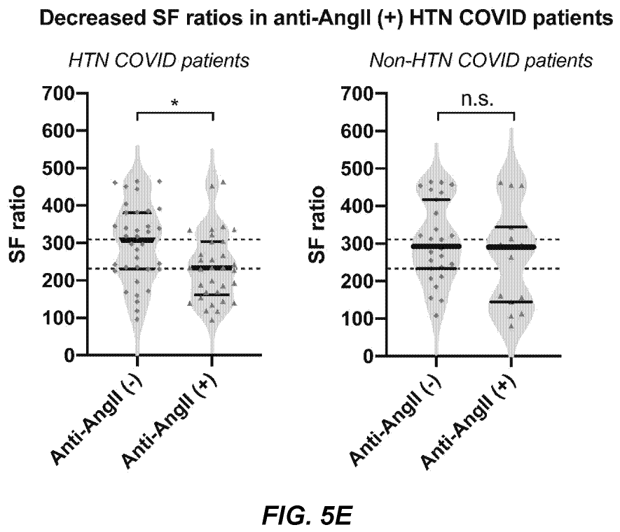

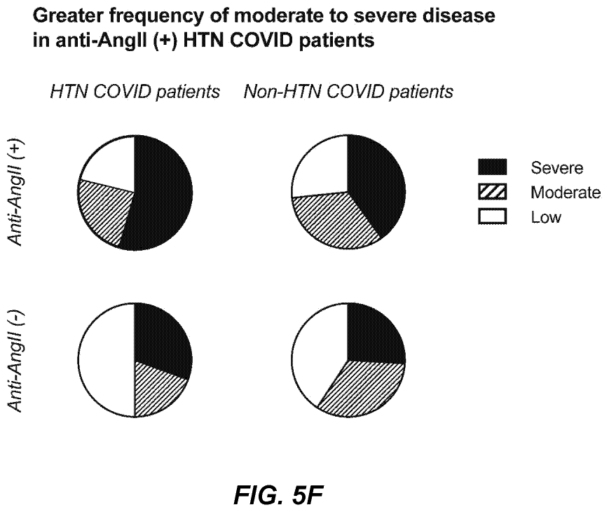

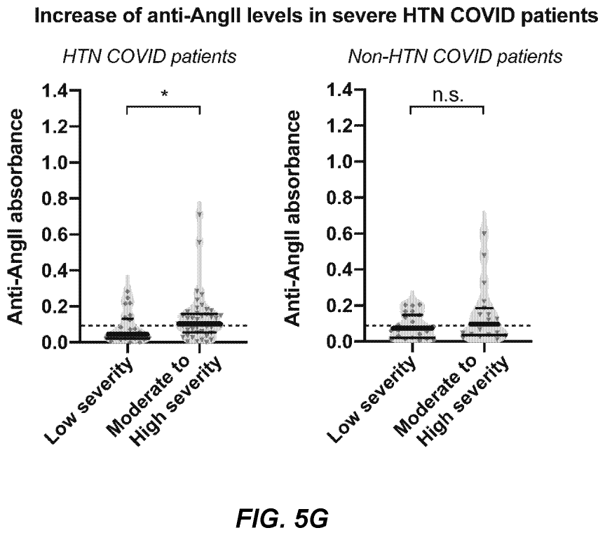

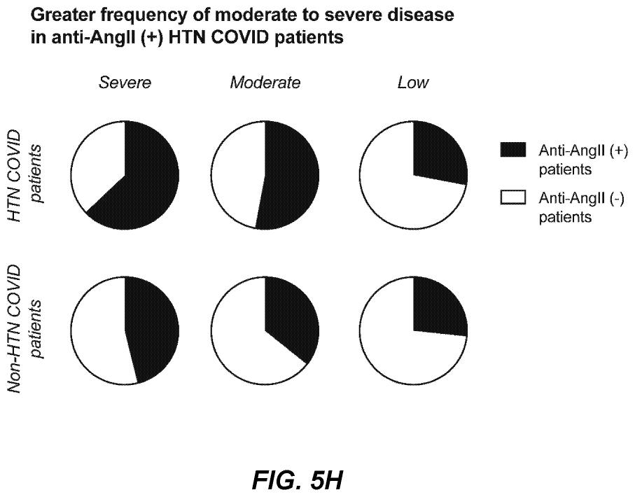

The inventors questioned whether the presence or levels of anti-AngII antibodies correlated with disease severity in COVID-19 patients as reflected by their daily median SF ratio, or pulse oximetric saturation, which reflects the ratio of peripheral oxygen saturation (SpO₂) to the fraction of inspired oxygen (FiO₂). Each anti-AngII measurement (as described in Example 1) was compared to the lowest daily mean SF value within a two-day window of that timepoint. For patients with multiple anti-AngII measurement timepoints, the inventors used the timepoint of highest disease severity or highest AngII level, whichever came first, and excluded late timepoints (>32 days) and patients for which time-matched SF data were not available. The corresponding lowest daily mean SF ratio was compared between the COVID-19 patients that developed anti-AngII auto-antibodies (48 patients) and those that did not (63 patients). The SF ratio was significantly reduced in anti-AngII (+) COVID patients (FIG. 5A), corresponding to increased clinical severity (low severity=SF>314; moderate severity=314>SF>235; high severity=SF<233). This corresponded to a significantly higher fraction of anti-AngII(+) patients (77%) having moderate or severe disease compared to AngII(−) patients (54%), as shown in FIG. 5B. Similarly, COVID-19 patients with moderate and severe disease exhibited higher average levels of anti-AngII autoantibodies (FIG. 5C), and the subset of anti-AngII(+) patients with severe (57%) and moderate (45%) disease were significantly higher than those of the anti-AngII(−) patients, of which 29% had severe and 25% had moderate disease (FIG. 5D).

Since AngII is a critical regulator of blood pressure, the inventors hypothesized that COVID-19 patients with pre-existing hypertension (HTN) might be more sensitive to the effects of anti-AngII antibodies, if they were sufficiently abundant to alter the angiotensin signaling cascade. They therefore split the patients into two cohorts, HTN and non-HTN, and found that the correlations between anti-AngII positivity and clinical severity were stronger in HTN vs non-HTN COVID-19 patients. Of the 69 COVID-19 patients with HTN, the mean SF ratios were significantly lower in the anti-AngII(+) subset (33 patients) as compared to the anti-AngII(−) subset (36 patients) (FIG. 5E). In the non-HTN COVID-19 patients (comprising 15 anti-AngII(+) and 27 anti-AngII(−) patients), that difference was less pronounced and lacked statistical significance (FIG. 5E), although this could have been due to the smaller population size. In addition, of all the HTN AngII(+) COVID-19 patients, significantly more had severe (55%) and moderate (24%) disease compared to HTN AngII(−) patients, where 50% had severe and 19% had moderate disease (FIG. 5F). These differences in clinical severity between the anti-AngII(+) vs anti-AngII(−) subsets were less pronounced in non-HTN COVID-19 patients, where of the AngII(+) subset, 40% were severe and 33% were moderate, while of the AngII(−) subset, 26% were severe and 33% were moderate (FIG. 5F). Finally, when subdividing HTN and non-HTN COVID-19 patients by severity rather than anti-AngII status, HTN COVID-19 patients with moderate to high severity showed higher average levels of anti-AngII antibodies compared to those with low severity, while in non-HTN patients, this difference was not statistically significant (FIG. 5G). Furthermore, the anti-AngII(+) fractions of severe (62%) and moderate (53%) HTN COVID-19 patients were higher than in non-HTN COVID-19 patients, where 46% of those with severe and 36% of those with moderate disease were anti-AngII(+) (FIG. 5H). Among the patients with low disease severity, similar fractions were AngII(+) between HTN and non-HTN patients (28% and 27%, respectively). Based on these results, the inventors concluded that the presence of anti-AngII auto-antibodies are correlated with COVID-19 disease severity, and that this correlation is stronger in patients with pre-existing hypertension.

### Example 5—SARS-CoV-2 Infection Induces Cross-Reactive Autoantibodies Against Angiotensin II

Patients infected with the severe acute respiratory syndrome coronavirus-2 (SARS-CoV-2) can experience life-threatening respiratory distress, blood pressure dysregulation and thrombosis. This is thought to be associated with an impaired activity of angiotensin-converting enzyme-2 (ACE-2), which is the main entry receptor of SARS-CoV-2 and which also tightly regulates blood pressure by converting the vasoconstrictive peptide angiotensin II (AngII) to a vasopressor peptide. Here, the inventors show that a significant proportion of hospitalized COVID-19 patients developed autoantibodies against AngII, whose presence correlates with lower blood oxygenation, blood pressure dysregulation, and overall higher disease severity. Anti-AngII antibodies can develop upon specific immune reaction to the SARS-CoV-2 proteins Spike or RBD, to which they can cross-bind, suggesting some epitope mimicry between AngII and Spike/RBD. These results provide important insights on how an immune reaction against SARS-CoV-2 can impair blood pressure regulation.

Severe acute respiratory syndrome coronavirus-2 (SARS-CoV-2), the causative virus of coronavirus disease 2019 (COVID-19), infects cells by binding to angiotensin-converting enzyme-2 (ACE-2) via the receptor-binding domain (RBD) of its Spike protein. ACE-2 is an enzyme expressed on the surfaces of alveolar epithelial cells and vascular endothelial cells (1), among others, that plays an important role in regulating blood pressure by converting the vasoconstrictive peptide angiotensin II (AngII) to the vasopressor peptide angiotensin-(1-7) (2, 3). It is known that SARS-CoV-2 infection can lead to dysregulation of vascular tension, endothelial inflammation and enhanced thrombosis, presumably through enhancing endocytosis of ACE-2 and thereby lowering its cell-surface presence (4, 5).

Here, the inventors sought to explore whether SARS-CoV-2 infection might induce auto-antibodies against the peptide AngII. The inventors hypothesized that the simultaneous binding of SARS-CoV-2 and AngII to ACE-2 might lead to their co-phagocytosis by antigen-presenting cells, thus providing a strong immune adjuvant (the virus molecules) to the self-peptide AngII, leading to an anti-AngII autoimmune response (6, 7). Moreover, the inventors asked whether some epitope mimicry might exist between a domain on the Spike protein and AngII, based on their shared binding to ACE-2. Importantly, the induction of anti-AngII antibodies in COVID-19 patients, if it occurs, could interfere with AngII processing by ACE2 and signaling to its receptors, potentially contributing to the dysregulation of vascular tension and worsening acute respiratory distress syndrome(8, 9).

The inventors conducted observational studies using serum of hospitalized COVID-19 patients and determined that such autoantibodies are indeed induced, independently of anti-RBD levels. Instead, their presence and levels were strongly correlated with blood pressure dysregulation and poor oxygenation. The inventors finally demonstrated cross-reactivity of some antibodies between AngII and the Spike protein, suggesting immune epitope homology between these molecules.

A. Results and Interpretation

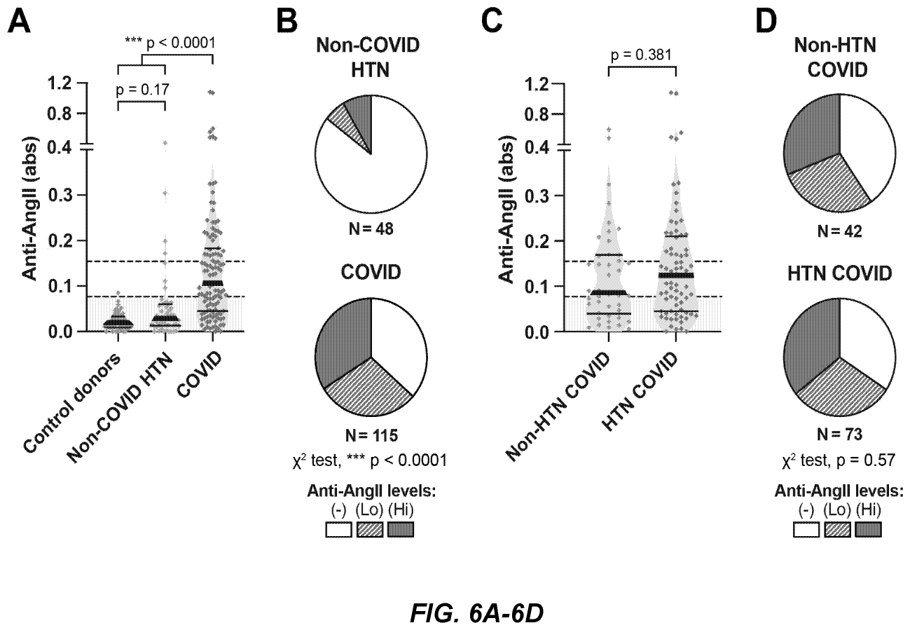

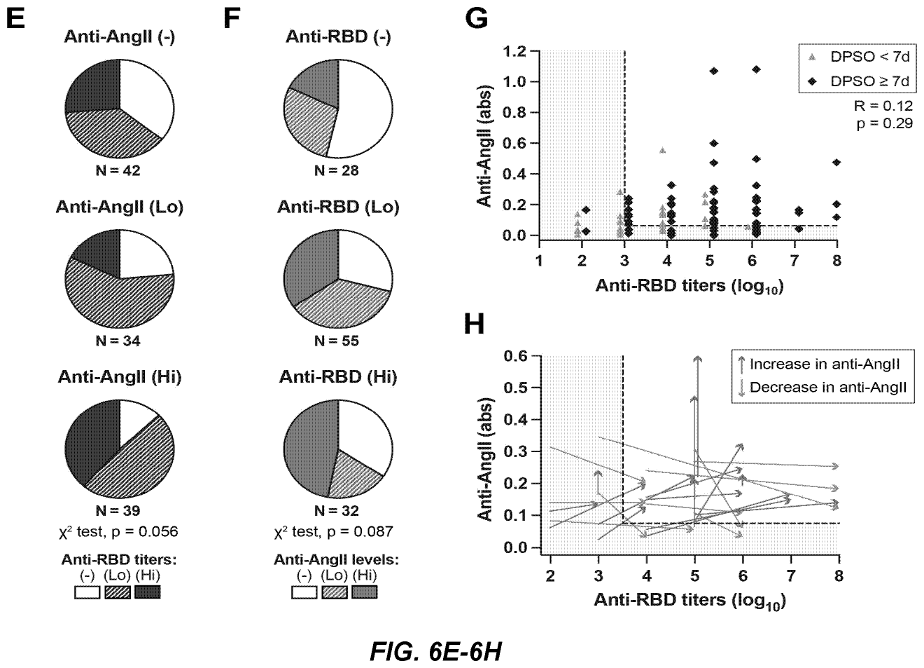

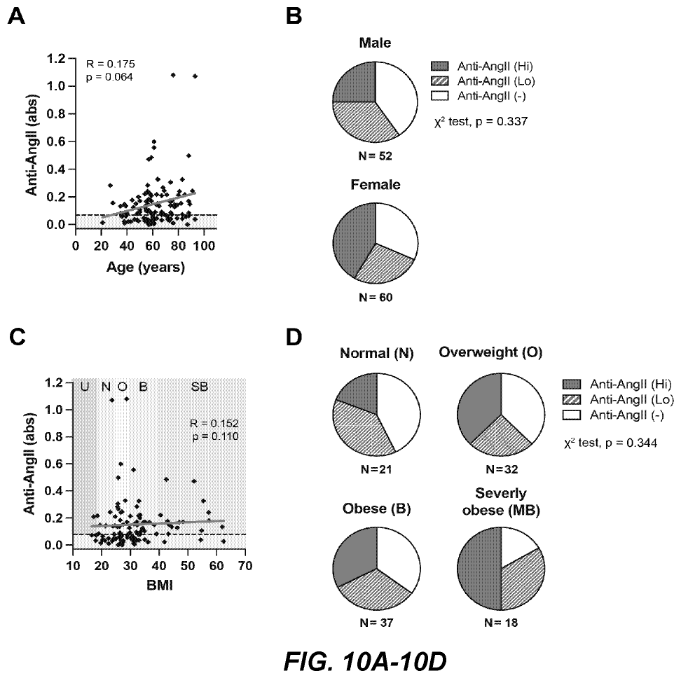

The inventors began by assessing the presence of IgG antibodies against AngII in the plasma of 221 subjects, among which 115 were hospitalized COVID-19 patients convalescent from a SARS-CoV-2 infection, 58 were control donors (i.e. non-SARS-CoV-2 infected and non-hypertensive), and 48 were hypertensive non-SARS-CoV-2 infected donors. Surprisingly, the inventors found that a substantial proportion, 63% (73/115), of the COVID-19 patients had positive levels of anti-AngII autoantibodies, as determined by an ELISA absorbance greater than 3 standard deviations above the mean of the control donor group (FIG. 6A). Of these, 53% (39/73) had high levels, defined as greater than twice the positive threshold. In contrast, only one control donor (1.7%) was anti-AngII positive, with a level just above the positive limit. Patient age, sex, and body mass index (BMI) were not significantly correlated with anti-AngII positivity (FIG. 10A-D), although older patients trended towards increased levels of anti-AngII (p=0.064 by Spearman correlation, FIG. 10A). Additional trends suggested that female COVID-19 patients were more likely to develop high anti-AngII levels (42% vs. 25% of males, FIG. 10B) as well as patients with BMI≥25 (38% vs. 20% of patients with normal BMI, FIG. 10C, D).

Since a majority of the hospitalized COVID-19 patients in this cohort had pre-existing hypertension (COVID HTN, 64%), the inventors measured anti-AngII levels in plasma from hypertensive donors taken prior to the pandemic (non-COVID HTN) to determine whether autoantibodies against AngII could have been pre-existing in HTN patients. Interestingly, the inventors detected anti-AngII antibodies in 15% of these hypertensive donors (FIG. 6A, B), which was far less than in COVID-19 patients. Furthermore, when comparing the HTN vs. non-HTN COVID-19 patients, the levels of anti-AngII were similar (FIG. 6C) as well as the percentages with positive levels (65% and 60%, respectively; FIG. 6D). These results indicate that infection with SARS-CoV-2 promotes the development of anti-AngII IgG antibodies in a majority of patients that developed severe disease (i.e., required hospitalization), regardless of whether they had pre-existing hypertension.

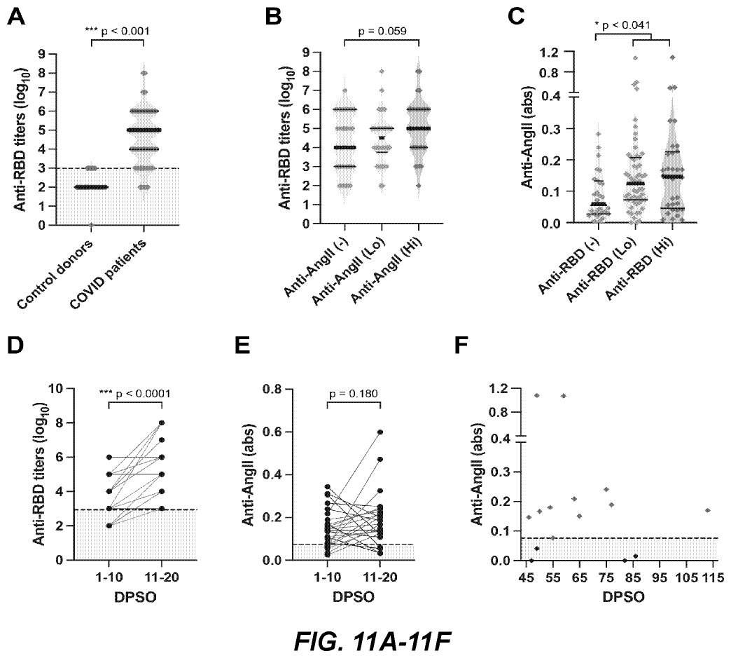

Next, the inventors asked whether the presence and levels of anti-AngII antibodies correlated with those of antibodies directed against the receptor-binding domain (RBD) of the virus' Spike protein. As expected, most patients (76%) developed positive levels of anti-RBD antibodies, here considered above a total IgG titer of 3 based on healthy control levels (FIG. 11A). However, correlations between anti-RBD levels and anti-AngII levels were only modest; in fact, a majority of anti-AngII(−) patients developed antibodies against RBD (FIG. 6E), and patients with high anti-RBD titers did not necessarily develop anti-AngII antibodies (34% remained anti-AngII(−)) (FIG. 6F). In addition, although patients positive for one antibody were more likely to be positive for the other, this effect was not statistically significant (p=0.056 and p=0.087 using χ² tests comparing the proportion of anti-AngII(+) patients across the anti-RBD (−/Lo/Hi) groups, and vice-versa). Similar results were observed when considering the average antibody levels instead of proportions of positive patients. Despite a slight increase in anti-AngII levels along with anti-RBD levels (FIG. 11B, 11C), no significant correlation was observed between them when analyzing the plasma of patients collected after 7 days post-symptom onset (DPSO), which is the approximate time needed to observe a new IgG response in blood upon primary infection (FIG. 6G, p=0.29 by Spearman test).

The inventors then questioned when anti-AngII antibodies developed relative to anti-RBD antibodies after SARS-CoV-2 infection by comparing their levels between early vs. later times (1-10 vs. 11-20 DPSO) in the 25 anti-AngII(+) patients for which these timepoints were available. From early to late, some patients showed increases in both anti-RBD and anti-AngII (FIG. 6H in red, 15/25), while others showed increases in anti-RBD accompanied by decreases in anti-AngII (FIG. 6H in blue, 10/25). Between these times, the increase in anti-RBD levels reached statistical significance (FIG. 11D), as expected, whereas the increase in anti-AngII levels did not (FIG. 11E, p=0.18 by Wilcoxon matched pairs sign rank test). Moreover, anti-AngII antibodies were still detected in 11 patients at >45 DPSO (FIG. 11F), suggesting that either anti-AngII antibodies are long-lasting, or that they are maintained over a long-lasting symptomatic period in these patients. Together, these data suggest that antibodies levels generated against AngII are not necessarily correlated with those against RBD.

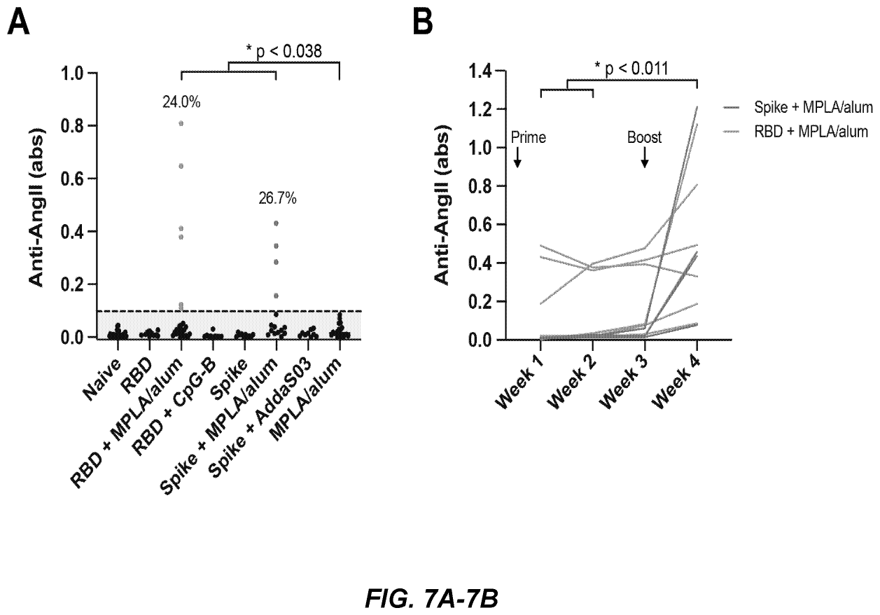

The inventors next studied the development of anti-AngII IgG in mice upon exposure to SARS-CoV-2 antigens (note that murine and human AngII are 100% homologous). The inventors vaccinated mice at 0 and 21 days with 10 μg Spike or RBD, admixed with either (i) the Toll-like receptor (TLR) 4 agonist monophosphoryl lipid A in alum (MPLA/Alum, mimicking the AS04 clinical adjuvant), (ii) AddaS03 (a formulation of composed of α-tocopherol, squalene and polysorbate 80 in an oil-in-water nano-emulsion, mimicking the AS03 clinical adjuvant), (iii) the TLR9 agonist CpG-B, or (iv) no adjuvant. None of the naïve mice (0/25) had detectable anti-AngII IgG (FIG. 7A). In contrast, 24% (6/25) of the mice vaccinated with RBD+MPLA/Alum and 27% (4/15) of mice vaccinated with Spike+MPLA/Alum developed antibodies against AngII. Interestingly, none of the mice vaccinated with Spike+AddaS03 (0/9) or with RBD+CpG (0/10) developed anti-AngII antibodies, as well as none of the mice vaccinated with unadjuvanted RBD or Spike (0/10 each), or with MPLA/Alum lacking antigen (0/20).

The inventors further analyzed the time course of the immune responses in mice, measuring anti-AngII levels at weeks 1, 2, 3 and 4 for the mice vaccinated with RBD+MPLA/alum or Spike+MPLA/Alum that were positive for anti-AngII. This analysis highlighted an increase in anti-AngII levels over time, specifically between weeks 1-2 and week 4 post-vaccination (FIG. 7B). Of note, the increase in anti-AngII IgG titers was more substantial after the vaccination boost at week 3. These results demonstrate that anti-AngII autoantibodies can be induced in mice by vaccination against Spike or RBD, in the absence of SARS-CoV-2 infection, at least with MPLA/Alum-containing adjuvants.

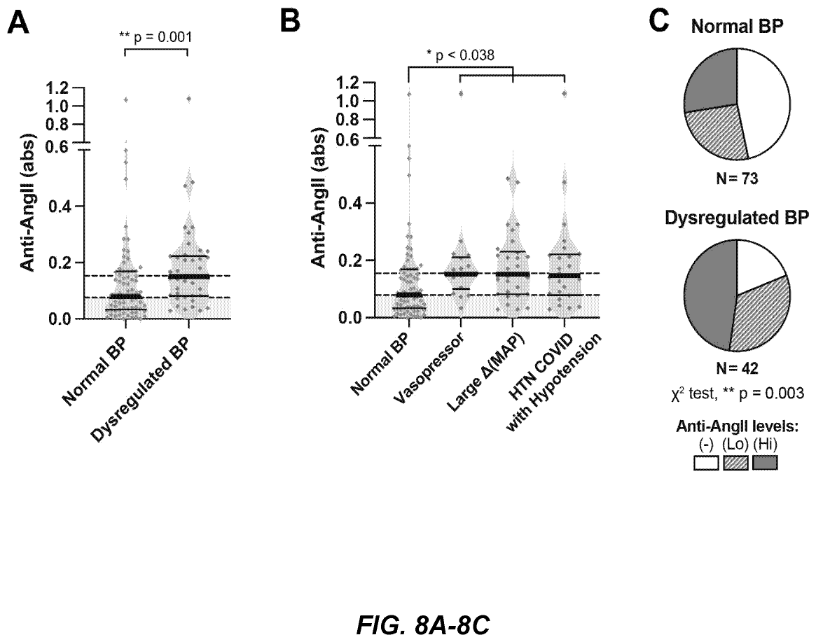

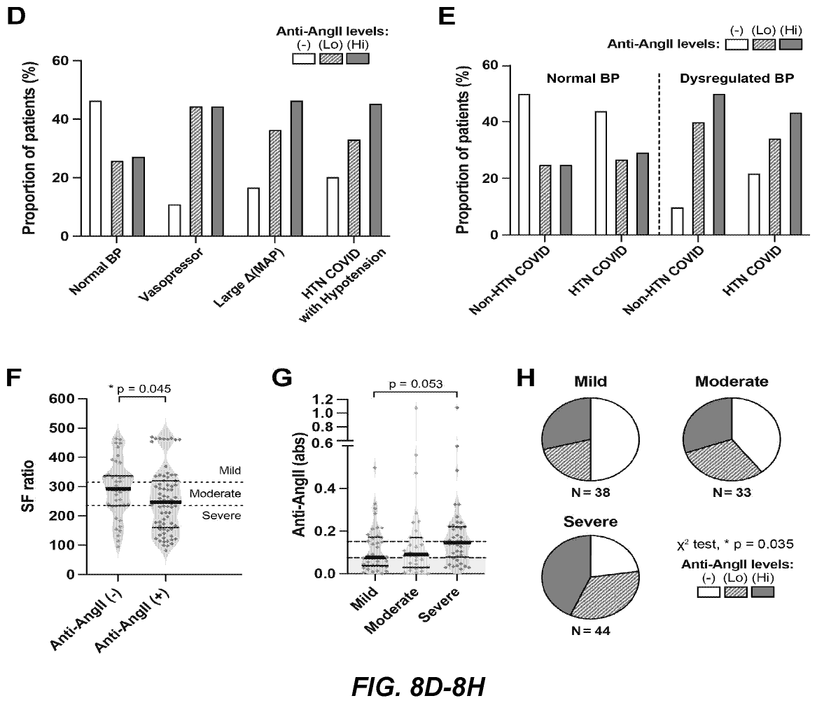

Having determined that anti-AngII antibodies could develop both in hospitalized COVID-19 patients and in mice after vaccination with MPLA/alum-adjuvanted SARS-CoV-2 antigens, the inventors further examined hospitalized COVID-19 patients for potential correlative effects of anti-AngII antibodies on blood pressure dysregulation, blood oxygenation, and disease severity. First, the inventors observed that the levels of anti-AngII were significantly higher in patients with dysregulated blood pressure (BP), as defined by (i) episodes of hypotension that required administration of vasopressors, (ii) large daily fluctuations in blood pressure (daily mean arterial pressure range (ΔMAP) ≥70 mmHg), or (iii) at least 2 consecutive days of hypotension (MAP <65 mmHg) in patients with pre-existing HTN (FIG. 8A). Moreover, this correlation remained significant when each of these subgroups were analyzed separately, despite the low number of patients available (FIG. 8B). Of note, these 3 subgroups of patients with dysregulated PB are not exclusive, with 52% of patients belonging to multiple categories. Consistent with these observations, a majority of patients with blood pressure dysregulation were positive for anti-AngII (81% vs. 53% in patients with normal blood pressure, FIG. 8C), among which 45% had high levels of anti-AngII. And finally, patients needing vasopressors were the most likely to have positive or high levels of anti-AngII, although among each of the groups with dysregulated blood pressure, >80% were anti-AngII(+) (FIG. 8D).

Since AngII is a critical regulator of blood pressure, the inventors further hypothesized that COVID-19 patients with pre-existing HTN might be more sensitive to the effects of anti-AngII antibodies, if they were sufficiently abundant to alter the angiotensin signaling cascade. Therefore, the inventors separated the patients with normal or dysregulated BP into subsets of patients with or without pre-existing HTN. Nevertheless, the inventors found no striking difference between the non-HTN vs. HTN subsets that could support enhanced effects of anti-AngII in COVID patients with pre-existing HTN (FIG. 8E).

Next, the inventors questioned whether the presence or levels of anti-AngII antibodies correlated with reduced blood oxygenation and disease severity, measured via the daily mean oximetric saturation (SF ratio), which is the ratio of peripheral oxygen saturation (SpO₂) to the fraction of inspired oxygen (FiO₂). For each patient, the inventors selected the lowest daily mean SF ratio value obtained within a two-day window of the measurement of anti-AngII. The lowest daily mean SF ratio was compared between the COVID-19 patients who developed auto-antibodies against AngII (73 patients) and those who did not (42 patients). The inventors found that the SF ratio was strongly reduced in anti-AngII(+) patients (FIG. 8F), demonstrating a correlation between anti-AngII antibodies and reduced blood oxygenation in these patients.

Since the clinical severity of COVID directly depends on the patient's blood oxygenation, patients with SF ratio ≥314 are considered as having a disease of low severity, while patients with SF ratio <235 suffer from a highly severe form of COVID. Based on this, the inventors then compared anti-AngII levels with disease severity and found that patients with severe vs. mild disease had increased anti-AngII antibodies (p=0.053, FIG. 8G). Similarly, the inventors found a significant increase in the proportion of anti-AngII(+) patients along with disease severity (p=0.035 by χ² test, FIG. 8H). Indeed, 77% of patients with severe disease were anti-AngII(+), including 41% with high levels, as compared to 50% and 29%, respectively, in patients with mild disease.

Together, these results demonstrate a correlation between the presence and levels of anti-AngII in COVID-19 patients and dysregulated blood pressure, lower blood oxygenation, and increased disease severity. While causal relationships cannot be drawn from these correlations, the fact that AngII is a key regulator of blood pressure in humans suggests that antibodies directed against anti-AngII could alter AngII signaling pathways, including its binding to and signaling via the angiotensin receptors AT1 and AT2. as well as on its enzymatic conversion by ACE2. Further experiments are needed to establish the pathophysiological consequences of anti-AngII auto-antibodies in COVID-19 patients.

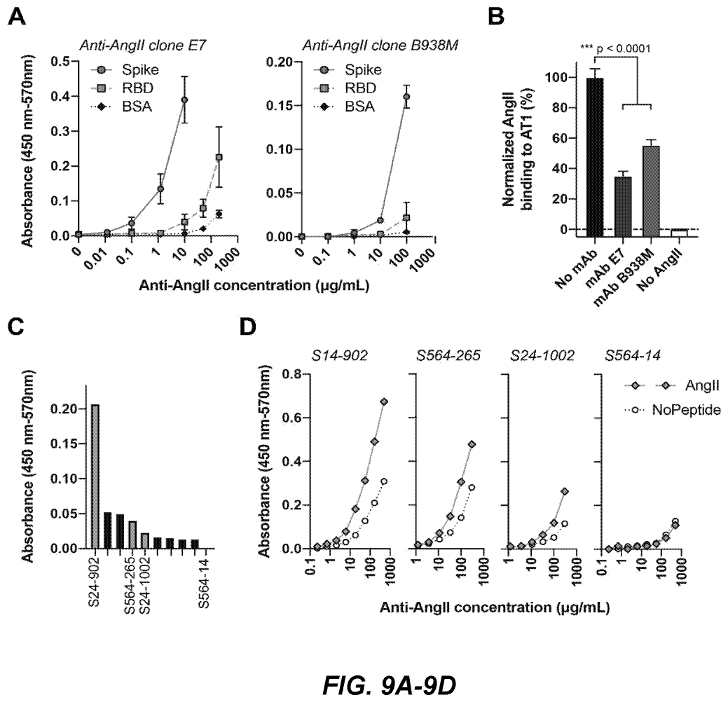

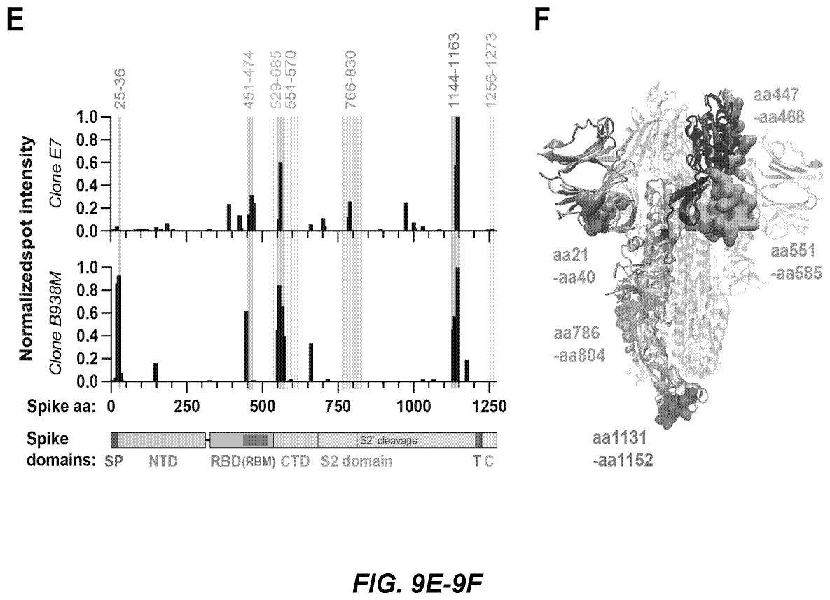

Because anti-AngII antibodies developed in response to SARS-CoV-2 infection in hospitalized patients or vaccination with some adjuvants in mice, the inventors hypothesized that anti-AngII antibodies may result from molecular structural mimicry between the AngII peptide and certain epitopes present in Spike or RBD. In that case, anti-AngII and anti-RBD could potentially cross-bind to both antigens. To test this hypothesis, the inventors assessed the binding of two murine IgG monoclonal anti-AngII antibodies (clone E7 and clone B938M) to recombinant SARS-CoV-2 Spike or RBD. The inventors found that both monoclonal anti-AngII antibodies bound to Spike and RBD, although with a lower affinity to RBD than to Spike (FIG. 9A). The inventors further demonstrated that the monoclonal anti-AngII antibodies may interfere with AngII binding to its cognate receptor AT1, suggesting that AngII-AT1 signaling could be modulated by the presence of anti-AngII (FIG. 9B).

Conversely, the inventors evaluated the ability of monoclonal anti-RBD antibodies, in a library of those isolated from SARS-CoV-2 infected patients, to bind to AngII. Among the 36 different monoclonal anti-RBD antibodies assessed, 9 showed some low to very-low binding to AngII, with one being superior to the others, namely clone S24-902 (FIG. 9C). The inventors further confirmed the significant although low binding of 3 of these anti-RBD to AngII, respectively S24-902, S564-265 and S24-1002, by characterizing their affinity at various concentrations (FIG. 9D). In contrast, clone S564-14 did not show any specific binding to AngII and was used as a negative control. It is not surprising that the cross-reactivity of anti-RBD to AngII is low, considering that monoclonal anti-AngII also binds weakly to RBD (FIG. 9A) and that AngII is a short 8-amino acid peptide.

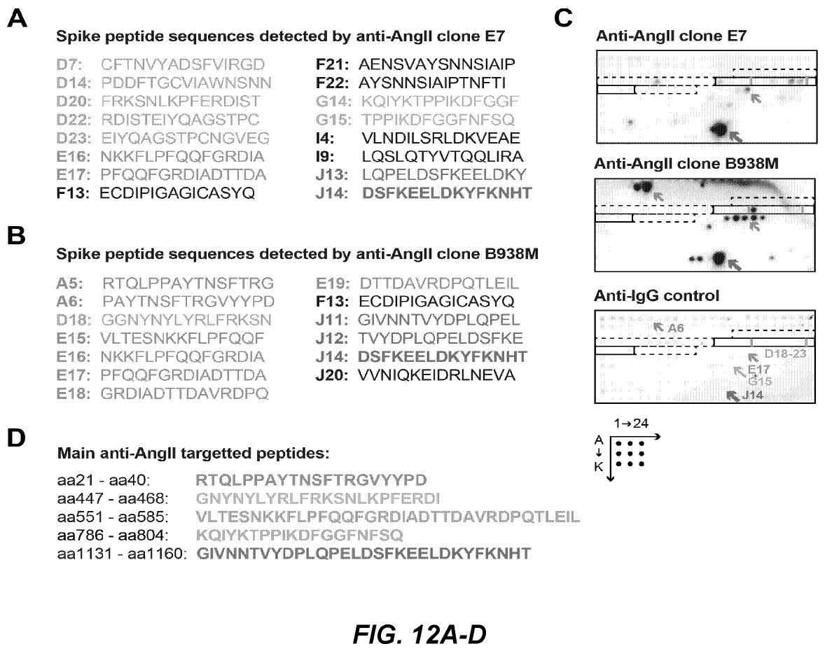

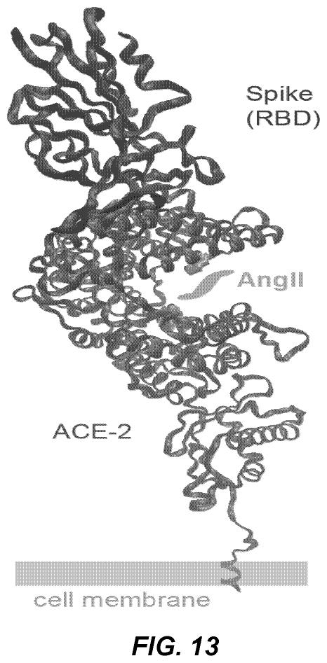

The inventors then sought to identify the epitopes of Spike or RBD that lead to cross-reactivity to AngII. To do so, the inventors tested the binding of the two monoclonal anti-AngII antibodies (clones E7 and B938M) to linear epitopes of Spike, using a peptide array that consists of a library of 15-mer peptides, shifted by 5 amino acids, covering the full-length of Spike. As a control for nonspecific binding, the inventors used the secondary antibody only, in the absence of anti-AngII antibody. Both anti-AngII E7 and B938M clones bound to several linear epitopes on Spike, with a main target at Spike residues aa1146-aa1160 located near the C-terminus, outside of RBD (FIG. 9E, FIG. 12A-C, spot J14 on the peptide array), and about 15 secondary targets of lower affinity. Five of the secondary peptides targeted by the clone E7 belong to the RBD of Spike, including 3 more particularly to the receptor-binding motif (RBM) of the RBD (FIG. 9E, FIG. 12A). In contrast, only 1 peptide targeted by the B938M clone belongs to the RBD region, in the RBM (FIG. 9E, FIG. 12B). Of note, although both Spike and AngII share the same receptor, ACE-2, their binding on ACE-2 occurs at 2 distinct locations (FIG. 13) (10, 11). Importantly, most regions targeted by clones E7 and/or B938M have been characterized as being the main B cells epitopes in COVID patients (colored regions in FIG. 9E, F, FIG. 12D) (12, 13). These regions, which are likely to contain the epitopes generating anti-AngII autoantibodies, are (1) aa21-aa40, (2) aa447-aa468, (3) aa551-aa585, (4) aa786-aa804, (5) aa1131-1160 of Spike.

The inventors next compared the Spike epitopes targeted by polyclonal responses raised against RBD/Spike in vaccinated mice or in COVID patients to the ones targeted by the monoclonal anti-AngII. Thus, the inventors repeated the peptide array assays using anti-AngII(+) or anti-AngII(−) pooled sera from 5 different mice vaccinated with Spike+MPLA/alum or RBD+MPLA/alum, or from 5 different COVID-19 patients.

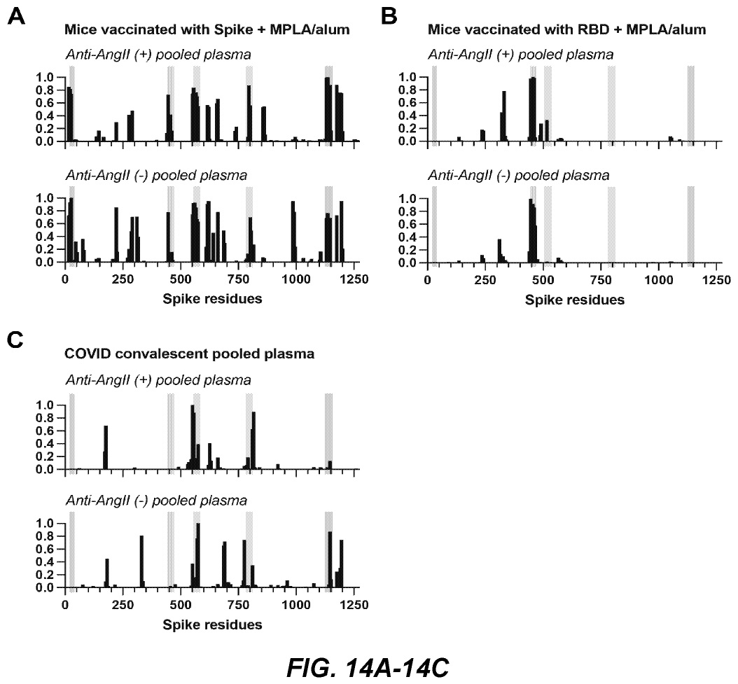

The inventors observed that mice raised anti-Spike/RBD antibodies in all regions previously targeted by the monoclonal anti-AngII upon vaccination with Spike+MPLA/alum (FIG. 14A), while mostly in the region aa447-aa468 upon vaccination by RBD+MPLA/alum (FIG. 14B). The profiles of anti-AngII(+) and anti-AngII(−) mice were very similar, except for a small increase in binding of anti-Spike to the region aa447-aa468 and anti-RBD to aa551-585, in anti-AngII(+) mice. As to the COVID-19 patients, they raised anti-Spike antibodies against 3 out of the 5 anti-AngII-targeted regions, namely aa551-aa585, aa786-aa804 and aa1131-1160, with a slightly higher binding observed to aa786-aa804 in anti-AngII(+) plasma (FIG. 14C). Surprisingly, the region aa1131-1160 was better targeted in anti-AngII(−) plasma; this highlights that not every antibody that bind to anti-AngII-targeting regions would necessarily cross-react with AngII.

Taken together, using monoclonal anti-AngII and anti-RBD antibodies, the inventors demonstrate that antibody cross-binding between AngII and Spike/RBD can occur, even if weakly, which may suggest some structural homology between AngII and certain epitopes of Spike/RBD.

SARS-CoV-2 infection has been shown to induce broad immune dysregulation (15, 16), including generation of wide-ranging autoantibody responses (17). For example, generation of autoantibodies against phospholipids has been shown to contribute to coagulation disorders (18, 19), and against Type I interferons to reduced immune response to infection (20). Here, the inventors show that autoantibodies can be generated against AngII, a key regulator of vascular tension. The inventors further show that generation of anti-AngII autoantibodies correlated with disease severity as reflected in dysregulation of vascular tension and lower blood oxygenation. This, along with the in vitro signaling data, suggests that the anti-AngII autoantibodies, even if low affinity, may be able to interfere with signaling between AngII and its receptors AT1 and ACE-2. Interestingly, some COVID-19 patients had also been shown to develop autoantibodies against AT1 and ACE-2, which similarly correlated with enhanced pro-inflammatory responses and increased disease severity (21, 22). Such autoantibodies against AT1 and ACE-2 have been also observed in patients suffering from other vascular pathologies, particularly in malignant hypertension (8) or constrictive vasculopathy (9). Therefore, systematic quantification of autoantibodies against key molecules of the renin-angiotensin pathway (i.e. AngII, AT1, and ACE-2), in diseases characterized by vascular dysregulation, including COVID-19, might reveal a common underlying autoimmune etiology. Lastly, the inventors highlighted the immune epitopes of Spike that could share structural homology with AngII, providing molecular insights in the immune mechanisms that could lead to the generation of anti-AngII autoantibodies upon infection by SARS-CoV-2.

B. Materials and Methods

1. Human Plasma Biobank and Clinical Data Collection

All samples collection and data recording on COVID-19 patients were approved by the IRB, under an informed consent, from a cohort at the University of Chicago. Serum samples were also collected from both non-hypertensive and hypertensive organ donors provided by the Gift of Hope Organ Donor Network (Itasca, IL). Patients of the COVID cohort were hospitalized patients, in contrast to non-SARS-CoV-2 infected healthy and hypertensive individuals, which were non-hospitalized donors. In addition, many of the COVID-19 patients had pre-existing health conditions, including hypertension. Blood was analyzed for the presence and amounts of specific anti-RBD IgG and anti-AngII IgG antibodies by enzyme-linked immunosorbent assay (ELISA).

2. Titration of Anti-RBD Antibodies

All procedures utilizing human samples were performed under Biosafety level 2 conditions. For the titration of anti-RBD antibodies, ELISA plates were coated with purified recombinant RBD (100 nM) in PBS for 12-16 hours at 4° C. Plates were then blocked with bovine serum albumin (BSA, 2%) in PBS for 2 hours at room temperature. Plates were then extensively washed with PBS-Tween 0.05% (PBST). Human plasmas were diluted at 1:100 in PBST+BSA (0.5%), and then serially diluted by 10. Samples were added to the plates and incubated for 2 hours at room temperature. Plates were washed again and incubated with a horseradish peroxidase (HRP)-conjugated anti-human IgG for 1 hour at room temperature in the dark. Plates were washed, revealed using 3,3′,5,5′-Tetramethylbenzidine (TMB) for 30 min in the dark, and the reaction was stopped using 1M H₂SO₄. Absorbances at 450 nm were read using an Epoch plate reader (BioTek), and corrected with the absorbance at 570 nm. Titers represent the highest dilution at which antibodies were detected, in log 10 (e.g. an IgG titer of 4 indicates a detectable level of anti-RBD IgG in a plasma sample diluted at 1:10000). A value of 0 was given to samples that had no detectable IgG at a dilution of 1:100. Anti-RBD titers are reported in log₁₀. Patients were considered positive for anti-RBD when their titers was ≥4, since some healthy donors samples taken prior to the COVID-19 pandemic had titers of 3. Anti-RBD titers were considered low at 4-5 and high at 5-8.

3. Detection of Anti-AngII Antibodies

For the detection of anti-AngII antibodies, streptavidin-coated plates were purchased from Pierce (ThermoFisher Scientific) and biotin-LC-AngII was purchased from AnaSpec. Streptavidin-coated plates were re-hydrated using PBST, and biotin-LC-AngII (10 μg/mL) was added to the plate for 2 hours at room temperature. Plates were washed with PBST, and plasma samples diluted at 1:100 were added to the plates for 2 hours at room temperature. Plates were washed again and incubated with an HRP-conjugated anti-human IgG for 1 hour at room temperature in the dark. Plates were washed and developed as previously described, except that the signals were stopped after about 10-15 min (when the absorbance of the reference well reached ˜1 AU). Absorbances at 450 nm were corrected with the absorbance at 570 nm. Detection of antibodies in absence of biotin-LC-AngII was also performed and subtracted to correct for AngII-unspecific antibody binding. Threshold to define positive signals is 3 standard deviations above the mean of the healthy donors cohort (after removal of the outliers according to the IQR rule) which corresponds to an absorbance of 0.077. Threshold to define high levels of anti-AngII is twice the threshold for positivity, which is an absorbance of 0.154.

4. Data Analysis of Anti-AngII and Anti-RBD in COVID Patients

Many patients had several plasma samples available at different days post-symptoms onset (DPSO). Anti-AngII and anti-RBD levels were measured for all available timepoints. As a rule, the highest value of anti-AngII for each patient was selected for data analysis, along with its associated anti-RBD titers at the same timepoint. The analysis of the anti-AngII/anti-RBD levels changes between early and late timepoints was performed on the 25 patients that were positive for anti-AngII and had samples availables for both time ranges in 1-10 and 11-21 DPSO. Again, the highest anti-AngII values in 1-10 and 11-21 DPSO were selected when multiple timepoints were available, and the anti-RBD corresponded to corresponding timepoints of highest anti-AngII. For the analysis at >45 DPSO, the highest anti-AngII value detected after 45 DPSO was similarly selected when more than one sample was available for a patient.

The categories for the body mass index (BMI) analysis were chosen according to the Center for disease control and prevention (CDC) definitions: underweight=BMI <18.5; normal=BMI between 18.5 and <25; overweight=BMI between 25 and <30; obese=BMI between 30 and <40; and severly obese=BMI ≥40.

5. Data Analysis of Anti-AngII Correlations to Blood Pressure Dysregulation, Blood Oxygenation and Disease Severity

Patients that were considered having a dysregulated blood pressure were: (1) the ones that received vasopressive drugs at any time during their hospitalization, (2) the ones that had large daily fluctuation of mean arterial pressure range ΔMAP ≥70 mmHg or (3) the ones that had pre-existing hypertension (HTN) and experienced at least 2 consecutive days of acute hypotension (MAP <65 mmHg). For the patients in (2) and (3), the dysregulation of blood pressure was assessed in the ±3 days around the timepoint of the highest anti-AngII value of the patient. Similarly, the SF ratio selected for each patient was the lowest SF ratio value detected in the ±3 days around the timepoint of the patient's highest anti-AngII value. Disease severity was defined according to the SF ratio value selected for each patient as such: mild disease=SF ratio ≥315; moderate disease=SF ratio between 235 and <315; severe disease=SF ratio <235.

6. Protein Productions

The ectodomain of Spike (BEI Resources: NR-52310) or the subdomain RBD (BEI Resources: NR-52309) were cloned into pCAGGS vector for mammalian expression in human embryonic kidney (HEK) 293F suspension cells, as his-tagged recombinant proteins. The proteins were expressed for 4-7 days in FreeStyle medium at 37° C., 5% CO₂, under constant agitation. Cell supernatants were then collected and filtered at 0.22 μm. The proteins were purified by immobilized nickel-affinity chromatography (HisTrap HP column, GE Healthcare) using fast protein liquid chromatography (FPLC; Äkta Pure system, GE Healthcare). Some protein batches were additionally purified by size exclusion chromatography (Superdex 200 pg column, GE Healthcare), and/or added with dithiothreitol (DTT) 10 mM to reduce protein dimerization. Proteins were then extensively dialyzed against phosphate-buffered saline (PBS; pH 7.4), sterile-filtered and stored at −80° C. All proteins were tested for endotoxin level <2 EU/mg.

7. Vaccines Formulation

Vaccines were formulated in PBS, by mixing 10 μg SARS-CoV-2 antigens, i.e. RBD or Spike, with various adjuvants. MPLA/alum was prepared by mixing 5 μg of MPLA (InvivoGen) with 50 μg of Alum (InvivoGen). AddaS03 was prepared by mixing 25 μL of AddaS03™ (Invivogen) with 25 μL of PBS containing the SARS-CoV-2 antigens. Mouse specific stimulatory CpG class B (ODN1826; InvivoGen) was injected at a dose of 20 μg. Non-adjuvanted groups were composed of 10 μg of antigen only. Mice vaccinated with MPLA/alum without antigens were injected with 5 μg of MPLA and 50 μg of Alum.

8. SARS-CoV-2 Vaccination and Analysis in Mice

All mouse experimentation was approved by the IACUC at the University of Chicago. Female 8-week old C57BL/6 mice received a prime vaccination using the vaccine formulations above-described, as well as a boost vaccination 3 weeks later. Vaccinations were administered by intradermal injections in the animal hocks in the two forelimbs (25 μL/hock). Mice were bled weekly via the submandibular vein after vaccination for plasma collection and analysis. Anti-AngII IgG levels were determined as described above, except using an anti-Ms total IgG as a detection antibody (1:8000; Southern Biotech). Threshold to define positive signals is 3 standard deviations above the mean of the group injected with MPLA/alum without SARS-CoV-2 antigen, which is an absorbance of 0.095.

9. Binding of Monoclonal Anti-AngII Antibodies to RBD and Spike

A mouse monoclonal IgG2a anti-AngII/III clone E7 (MA1-82996) was purchased from ThermoFisher Scientific and a mouse monoclonal IgG1 anti AngI/II/III (GTX44411) was purchased from GeneTex. ELISA plates were coated with RBD or Spike (100 nM) in PBS for 12-16 hours at 4° C. The plate was then blocked using BSA 2% for 2 hours at room temperature. The anti-AngII antibody was diluted in PBST-BSA (0.5%) at the specified concentrations and added to the plate for 2 hours at room temperature. Binding of the anti-AngII to RBD, Spike or BSA was detected using an anti-mouse IgG (Southern Biotech) for 1 hour at room temperature in the dark. The plate was then revealed and the absorbance determined as described above.

10. Inhibition of AngII Binding to AT1 by Anti-AngII Monoclonal Antibodies

Binding of AngII to AT1 receptors was assessed using fluorescently labelled FAM-AngII, purchased from AnaSpec, and Chinese Hamster Ovarian (CHO) cells expressing recombinant human AT1 (CHO-AT1 cells), purchased from Perkin-Elmer. FAM-AngII (100 nM) was pre-incubated with 0.3 mg/mL of the anti-AngII monoclonal antibodies (clone E7 or B938M) in PBS for 30 min at 37° C. or with no antibody as a negative control. Then, CHO-AT1 cells were stained with these mixtures in the dark for 20 min on ice. Cells were washed 3 times in PBS+2% Fetal Bovine Serum, after what the binding of FAM-AngII to AT1 on the cell surface was detected using a flow cytometer (BD LSRFortessa, BD Biosciences). The mean fluorescence intensity (MFI) of the FAM-AngII was computed using FlowJo software.

11. Binding of Monoclonal Anti-RBD to AngII

A library of monoclonal anti-RBD was isolated and sequenced from COVID-19 patients, produced as recombinant proteins using HEK-293 cells and purified using Protein-G affinity-based purification. The binding of the monoclonal anti-RBD to AngII was assessed as described above, by coating streptavidin-coated plate with 10 μg/mL biotin-LC-AngII, washing with PBST, incubating with anti-RBD clones at 20 μg/mL or indicated concentrations, washing again, and detecting with an anti-human IgG. Negative controls were done in absence of biotin-LC-AngII.

12. Peptide Array of SARS-CoV-2 Spike

SARS-CoV-2 Full Spike CelluSpots peptide array assays were purchased from Intavis Bioanalytical Instruments. The assay contains 254 peptides of 15 amino acids (aa) length covering the full-length Spike sequence with a shift of 5 aa between peptides. The assay was performed as instructed by the manufacturer. Briefly, the membrane was blocked with casein blocking buffer (Sigma-Aldrich) for 4 hours at room temperature, and then incubated for 12 hours at 4° C. with the anti-AngII clone E7 diluted at a concentration of 20 μg/mL in casein. The membrane was then washed extensively using PBST, and further incubated with an HRP-conjugated anti-mouse IgG (1:5000; Southern Biotech) for 2 hours at room temperature. The membrane was again extensively washed in PBST and revealed using the Clarity ECL Western substrate (BioRad). The membrane was imaged using a gel imager (BioRad) and spot intensity were analysed using the Protein Array Analyze for ImageJ (2010) made by Carpentier G. and available online at: http://rsb.info.nih.gov/ij/macros/toolsets/Protein Array Analyzer.txt. For peptide arrays using plasma from COVID-19 patients or vaccinated mice, the same procedure was followed, except that the pooled plasma from 5 individuals was diluted at 1:200 in casein and incubated for 4 h at room temperature instead of the incubation with anti-AngII antibodies. For patients samples, an anti-human IgG was used as a secondary antibody to reveal signals.

13. Statistics and Softwares

Graphs and statistical analysis were performed using Prism 9 (GraphPad Software LLC). Median±interquartile range are represented in violin plots, while mean±SD are represented in other types of graphs. Non-parametric tests (Mann-Whitney for 2 groups comparison, Wilcoxon for paired data, Kruskall-Wallis for >2 groups comparisons) were used for data with non-normal distribution. Spearman tests were used for correlations. ANOVA were used for multiple groups comparison on normally distributed data. All tests were double-sided and the p-values were corrected for multiple comparisons. Statistics for χ² proportion tests were performed online using https://www.socscistatistics.com calculators. χ² tests were used to compare the % of anti-AngII positive patients between the different categories of interest. Threshold for statistical significance was p<0.05.

Excel (Microsoft) was used to sort and analyze the data. Molecular structures visualization rendering were done using VMD (Visual Molecular Dynamics 1.9.1). Illustrator CS5 (Adobe) was used to make the figures.

All of the methods disclosed and claimed herein can be made and executed without undue experimentation in light of the present disclosure. While the compositions and methods of this invention have been described in terms of certain embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

1. K. Kuba, Y. Imai, T. Ohto-Nakanishi, J. M. Penninger, Trilogy of ACE2: a peptidase in the renin-angiotensin system, a SARS receptor, and a partner for amino acid transporters. Pharmacology and Therapeutics. 128, 119-128 (2010).
2. M. Bader, ACE2, angiotensin-(1-7), and Mas: the other side of the coin. Pflugers Arch. 465, 79-85 (2013).
3. F. Alhenc-Gelas, T. B. Drueke, Blockade of SARS-CoV-2 infection by recombinant soluble ACE2. Kidney Int. 97, 1091-1093 (2020).
4. L. Samavati, B. D. Uhal, ACE2, Much More Than Just a Receptor for SARS-COV-2. Front Cell Infect Microbiol. 10, 317 (2020).
5. P. Verdecchia, C. Cavallini, A. Spanevello, F. Angeli, The pivotal link between ACE2 deficiency and SARS-CoV-2 infection. Eur J Intern Med. 76, 14-20 (2020).
6. B. T. Rouse, S. Sehrawat, Immunity and immunopathology to viruses: what decides the outcome? Nat Rev Immunol. 10, 514-526 (2010).
7. J. Mercer, U. F. Greber, Virus interactions with endocytic pathways in macrophages and dendritic cells. Trends Microbiol. 21, 380-388 (2013).
8. M. L. X. Fu et al., Autoantibodies against the angiotensin receptor (AT1) in patients with hypertension. Journal of Hypertension. 18, 945 (2000).
9. Y. Takahashi, S. Haga, Y. Ishizaka, A. Mimori, Autoantibodies to angiotensin-converting enzyme 2 in patients with connective tissue diseases. Arthritis Research & Therapy. 12, R85-8 (2010).
10. R. Yan et al., Structural basis for the recognition of SARS-CoV-2 by full-length human ACE2. Science. 367, 1444-1448 (2020).
11. J. L. Guy, R. M. Jackson, H. A. Jensen, N. M. Hooper, A. J. Turner, Identification of critical active-site residues in angiotensin-converting enzyme-2 (ACE2) by site-directed mutagenesis. FEBS J. 272, 3512-3520 (2005).
12. E. Shrock et al., Viral epitope profiling of COVID-19 patients reveals cross-reactivity and correlates of severity. Science. 370 (2020), doi:10.1126/science.abd4250.
13. Y. Li et al., Linear epitope landscape of the SARS-CoV-2 Spike protein constructed from 1,051 COVID-19 patients. Cell Rep. 34, 108915 (2021).
14. A. C. Walls et al., Structure, Function, and Antigenicity of the SARS-CoV-2 Spike Glycoprotein. Cell. 181, 281-292.e6 (2020).
15. E. J. Giamarellos-Bourboulis et al., Complex Immune Dysregulation in COVID-19 Patients with Severe Respiratory Failure. Cell Host Microbe. 27, 992-1000.e3 (2020).
16. S. Bhattacharjee, M. Banerjee, Immune Thrombocytopenia Secondary to COVID-19: a Systematic Review. SN Compr Clin Med. 2, 1-11 (2020).
17. M. Zuniga et al., Autoimmunity to Annexin A2 predicts mortality among hospitalised COVID-19 patients. Eur Respir J (2021), doi:10.1183/13993003.00918-2021.
18. Y. Zuo et al., Prothrombotic autoantibodies in serum from patients hospitalized with COVID-19. Sci Transl Med. 12 (2020), doi:10.1126/scitranslmed.abd3876.
19. J. M. Connors, J. H. Levy, COVID-19 and its implications for thrombosis and anticoagulation. Blood. 135, 2033-2040 (2020).
20. P. Bastard et al., Autoantibodies against type I IFNs in patients with life-threatening COVID-19. Science. 370 (2020), doi:10.1126/science.abd4585.
21. A. I. Rodriguez-Perez et al., Autoantibodies against ACE2 and angiotensin type-1 receptors increase severity of COVID-19. Journal of Autoimmunity. 122, 102683 (2021).
22. L. Casciola-Rosen et al., IgM autoantibodies recognizing ACE2 are associated with severe COVID-19. medRxiv, 2020.10.13.20211664 (2020).

1. A method for evaluating a subject having or having had a coronavirus infection, the method comprising detecting, in a biological sample from the subject, an antibody that binds to angiotensin II.

2. A method for evaluating a subject having been administered a coronavirus vaccine, the method comprising detecting, in a biological sample from the subject, an antibody that binds to angiotensin II.

3. The method of claim 1 or 2, wherein the coronavirus is SARS-CoV-2.

4. The method of claim 3, wherein the antibody further binds to SARS-CoV-2 Spike protein.

5. The method of claim 3, wherein the antibody binds to a region of SARS-CoV-2 Spike protein comprising one of SEQ ID NOs: 1-16 or 22-40.

6. The method of claim 3 or 4, wherein the antibody binds to a region of SARS-CoV-2 Spike protein comprising SEQ ID NO:1.

7. The method of any of claims 1-6, further comprising obtaining the biological sample from the subject.

8. The method of claim 7, wherein the biological sample is a blood sample.

9. The method of claim 7, wherein the biological sample is a plasma sample.

10. The method of claim 7, wherein the biological sample is a serum sample.

11. The method of any of claims 1-9, wherein detecting the antibody comprises an ELISA.

12. The method of any of claims 1-9, wherein detecting the antibody comprises a lateral flow assay.

13. The method of any of claims 1-9, wherein detecting the antibody comprises a surface plasmon resonance assay.

14. The method of any one of claims 1-13, wherein the biological sample is from a subject that has hypertension.

15. The method of claim 14, wherein the hypertension comprises stage 1 hypertension, stage 2 hypertension, or hypertensive crisis.

16. The method of any one of claims 1-13, wherein the biological sample is from a subject that does not have hypertension, but has been diagnosed with hypertension in the past.

17. The method of any one of claims 1-15, wherein the subject is being treated for hypertension.

18. The method of claim 17, wherein the treatment comprises a diuretic, angiotensin-converting enzyme (ACE) inhibitor, angiotensin II receptor blocker (ARB), calcium channel blocker, or combinations thereof.

19. A method for treating or preventing a SARS-CoV-2 infection comprising administering to a subject a composition comprising (i) a portion of SARS-CoV-2 Spike protein or (ii) a nucleic acid encoding for a portion of SARS-CoV-2 Spike protein, wherein the portion of SARS-CoV-2 Spike protein does not comprise a sequence selected from the group consisting of SEQ ID NOs: 1-16 or 22-40.

20. The method of claim 19, wherein the portion of SARS-CoV-2 Spike protein does not comprise SEQ ID NO:1.

21. The method of claim 19, wherein the portion of SARS-CoV-2 Spike protein does not comprise SEQ ID NO:36.

22. The method of claim 19, wherein the portion of SARS-CoV-2 Spike protein does not comprise SEQ ID NO:37.

23. The method of claim 19, wherein the portion of SARS-CoV-2 Spike protein does not comprise SEQ ID NO:38.

24. The method of claim 19, wherein the portion of SARS-CoV-2 Spike protein does not comprise SEQ ID NO:39.

25. The method of claim 19, wherein the portion of SARS-CoV-2 Spike protein does not comprise SEQ ID NO:40.

26. The method of any of claims 19-25, wherein the composition further comprises a pharmaceutically acceptable excipient.

27. The method of any of claims 19-26, wherein the composition further comprises an adjuvant.

28. The method of any of claims 19-27, wherein the subject develops antibodies that bind to SARS-CoV-2 Spike protein.

29. The method of any of claims 19-28, wherein the subject does not develop antibodies that bind to angiotensin II.

30. The method of any one of claims 19-29, wherein the method further comprises evaluating the subject for the presence of angiotensin II autoantibodies in a biological sample from the subject.

31. A pharmaceutical composition comprising:
(a) (i) a portion of a SARS-CoV-2 Spike protein or (ii) a nucleic acid encoding for a portion of a SARS-CoV-2 Spike protein, wherein the portion of the SARS-CoV-2 Spike protein does not comprise a sequence selected from the group consisting of SEQ ID NOs: 1-16 or 22-40; and
(b) a pharmaceutically acceptable excipient.

32. The pharmaceutical composition of claim 31, wherein the portion of SARS-CoV-2 Spike protein does not comprise SEQ ID NO:1.

33. The pharmaceutical composition of claim 31, wherein the portion of SARS-CoV-2 Spike protein does not comprise SEQ ID NO:36.

34. The pharmaceutical composition of claim 31, wherein the portion of SARS-CoV-2 Spike protein does not comprise SEQ ID NO:37.

35. The pharmaceutical composition of claim 31, wherein the portion of SARS-CoV-2 Spike protein does not comprise SEQ ID NO:38.

36. The pharmaceutical composition of claim 31, wherein the portion of SARS-CoV-2 Spike protein does not comprise SEQ ID NO:39.

37. The pharmaceutical composition of claim 31, wherein the portion of SARS-CoV-2 Spike protein does not comprise SEQ ID NO:40.

38. The pharmaceutical composition of any of claims 31-37, further comprising an adjuvant.

39. A method for treating a subject for a SARS-CoV-2 infection comprising administering to the subject an effective amount of a SARS-CoV-2 treatment or a blood pressure regulating agent, wherein a biological sample from the subject was determined to comprise an antibody that binds to angiotensin II.

40. The method of claim 39, wherein the SARS-CoV-2 treatment comprises an anti-SARS-CoV-2 antibody.

41. The method of claim 39, wherein the SARS-CoV-2 treatment comprises COVID-19 convalescent plasma.

42. The method of claim 39, wherein the SARS-CoV-2 treatment comprises an anti-viral agent.

43. The method of claim 39, wherein the SARS-CoV-2 treatment comprises an aptamer.

44. The method of any one of claims 39-43, wherein the blood pressure regulating agent comprises a vasopressor.

45. The method of claim 44, wherein the vasopressor comprises epinephrine, droxidopa, isoproterenol, norepinephrine, phenylephrine, ephedrine, angiotensin II, or dobutamine.

46. The method of any one of claims 39-45, wherein the blood pressure regulating agent comprises a diuretic, angiotensin-converting enzyme (ACE) inhibitor, angiotensin II receptor blocker (ARB), calcium channel blocker, or combinations thereof.

47. The method of any one of claims 39-46, wherein the subject has been diagnosed with hypotension.

48. The method of claim 47, wherein the subject has been diagnosed with acute hypotension.

49. The method of any of claims 39-42, wherein the biological sample is a blood sample.

50. The method of any of claims 39-42, wherein the biological sample is a plasma sample.

51. The method of any of claims 39-42, wherein the biological sample is a serum sample.

52. The method of any of claims 39-51, wherein the subject has hypertension.

53. The method of any of claims 39-51, wherein the subject previously had hypertension.

54. A method for evaluating a subject comprising detecting, in a biological sample from the subject, an angiotensin II autoantibody.

55. The method of claim 54, wherein the subject has or has had a SARS-CoV infection.

56. The method of claim 54, wherein the subject has or has had a SARS-CoV-2 infection.

57. The method of claim 56, wherein the subject has a SARS-CoV-2 infection.

58. The method of claim 57, further comprising administering a SARS-CoV-2 treatment or blood pressure regulating agent to the subject.

59. The method of claim 58, wherein the SARS-CoV-2 treatment comprises an anti-SARS-CoV-2 antibody.

60. The method of claim 58, wherein the SARS-CoV-2 treatment comprises COVID-19 convalescent plasma.

61. The method of claim 58, wherein the SARS-CoV-2 treatment comprises an anti-viral agent.

62. The method of claim 58, wherein the SARS-CoV-2 treatment comprises an aptamer.

63. The method of claim 56, wherein the subject has had a SARS-CoV-2 infection.

64. The method of any of claims 54-63, wherein the biological sample is a blood sample.

65. The method of claim 64, wherein the biological sample is a plasma sample.

66. The method of claim 64, wherein the biological sample is a serum sample.

67. The method of claim 64-65, wherein the biological sample is a convalescent plasma sample.

68. The method of claim 64, wherein the biological sample is a convalescent serum sample.

69. The method of claim 54, wherein the subject has been administered a SARS-CoV vaccine.

70. The method of claim 54, wherein the subject has been administered a SARS-CoV-2 vaccine.

71. The method of claim 54, wherein the subject has or has had a cardiovascular disorder.

72. The method of claim 71, wherein the cardiovascular disorder is high blood pressure or heart failure.

73. The method of any of claims 54-72, wherein the antibody further binds to SARS-CoV-2 Spike protein.

74. The method of claim 73, wherein the antibody binds to a region of SARS-CoV-2 Spike protein comprising one of SEQ ID NOs: 1-16 or 22-40.

75. The method of claim 74, wherein the antibody binds to a region of SARS-CoV-2 Spike protein comprising SEQ ID NO:1.

76. The method of any of claims 54-75, further comprising obtaining the biological sample from the subject.

77. The method of claim 76, wherein the biological sample is a plasma sample.

78. The method of claim 76, wherein the biological sample is a serum sample.

79. The method of any of claims 54-78, wherein detecting the antibody comprises an ELISA.

80. The method of any of claims 54-78, wherein detecting the antibody comprises a lateral flow assay.

81. The method of any of claims 54-78, wherein detecting the antibody comprises a surface plasmon resonance assay.

82. A method for screening a SARS-CoV-2 therapeutic composition comprising detecting, in the therapeutic composition, an antibody that binds to angiotensin II.

83. The method of claim 82, wherein the SARS-CoV-2 therapeutic composition comprises COVID-19 convalescent plasma.

84. The method of claim 82, wherein the SARS-CoV-2 therapeutic composition comprises an anti-SARS-CoV-2 Spike protein antibody.

85. The method of claim 82, wherein the SARS-CoV-2 therapeutic composition comprises an aptamer.
